(19) United States
(12) Patent Application Publication
(10) Pub. No.: US 2006/0135430 A1
(43) Pub. Date: Jun. 22, 2006

(54) BLy antagonists and uses thereof

(75) Inventors: Andrew Chen-Yuen Chan, Menlo Park, CA (US); Nathaniel C. Gordon, Berkeley, CA (US); Robert F. Kelley, San Bruno, CA (US); Michael F.T. Koehler, Burlingame, CA (US); Melissa A. Starovasnik, San Francisco, CA (US)

Correspondence Address: MERCHANT & GOULD PC, P.O. BOX 2903, MINNEAPOLIS, MN, 55402-0903, US

(73) Assignee: GENENTECH, INC., SOUTH SAN FRANCISCO, CA (US)

(21) Appl. No.: 11/291,698

(22) Filed: Nov. 30, 2005

Related U.S. Application Data
(63) Continuation of application No. PCT/US04/17682, filed on Jun. 4, 2004.
(60) Provisional application No. 60/476,414, filed on Jun. 5, 2003.
(60) Provisional application No. 60/476,531, filed on Jun. 6, 2003.
(60) Provisional application No. 60/476,481, filed on Jun. 5, 2003.

(51) Int. Cl. A61K 38/08; C07K 7/08; A61K 38/10

(52) U.S. Cl. 514/13; 530/326

(57) ABSTRACT

The present invention relates to polypeptides that block BLyS signaling, nucleic acid molecules encoding the polypeptides, and compositions comprising the polypeptides. The present invention also relates to methods for treating an immune-related disease or cancer using the polypeptides and compositions of the invention. The present invention also relates to methods for identifying inhibitors of BLyS signaling.

### CROSS-REFERENCE

This application claims benefit from: U.S. Provisional Application Ser. No. 60/476,414, filed Jun. 5, 2003; U.S. Provisional Application Ser. No. 60/476,531, filed Jun. 6, 2003; and U.S. Provisional Application No. 60/476,481, filed Jun. 5, 2003.

### FIELD OF THE INVENTION

The present invention relates to polypeptides that inhibit BLyS signaling, nucleic acid molecules encoding the polypeptides and compositions comprising them. The present invention also relates to methods for preventing and treating immune related diseases and cancer using the compositions of this invention. The present invention also relates to methods for selecting inhibitors of BLyS signaling using the polypeptides of this invention.

### BACKGROUND AND INTRODUCTION OF THE INVENTION

BLyS (also known as BAFF, TALL-1, THANK, TNFSF13B, or zTNF4), is a member of the tumor necrosis family (TNF) superfamily of ligands, and is a crucial survival factor for B cells. BLyS overexpression in transgenic mice leads to B cell hyperplasia and development of severe autoimmune disease (Mackay, et al. (1999) J. Exp. Med. 190, 1697-1710; Gross, et al. (2000) Nature 404, 995-999; Khare, et al. (2000) Proc. Natl. Acad. Sci. U.S.A. 97, 3370-33752-4). BLyS levels are elevated in human patients with a variety of autoimmune disorders, such as systemic lupus erythematosus, rheumatoid arthritis, and Sjögren's syndrome (Cheema, G. S, et al., (2001) Arthritis Rheum. 44, 1313-1319; Groom, J., et al, (2002) J. Clin. Invest. 109, 59-68; Zhang, J., et al., (2001) J. Immunol. 166, 6-10). Furthermore, BLyS levels correlate with disease severity, suggesting that BLyS can play a direct role in the pathogenesis of these illnesses.

BLyS binds three receptors, TACI, BCMA, and BR3, with signaling through BR3 being essential for promoting B cell function. Of the three receptors to which BLyS binds, only BR3 is specific for BLYS; the other two also bind the related TNF family member, APRIL. Comparison of the phenotypes of BLyS and receptor knockout or mutant mice indicates that signaling through BR3 mediates the B cell survival functions of BLyS (Thompson, J. S., et al., (2001) Science 293, 2108-2111; Yan, M., et al., (2000) Nat. Immunol. 1, 37-41; Schiemann, B., et al., (2001) Science 293, 2111-2114). In contrast, TACI appears to act as an inhibitory receptor (Yan, M., (2001) Nat. Immunol. 2, 638-643), while the role of BCMA is unclear (Schiemann, supra).

BR3 is a 184-residue type III transmembrane protein expressed on the surface of B cells (Thompson, et al., supra; Yan, (2002), supra). The intracellular region bears no sequence similarity to known structural domains or protein-protein interaction motifs. BLyS-induced signaling through BR3 results in processing of the transcription factor NF-B2/p100 to p52 (Claudio, E, et al., (2002) Nat. Immunol. 3, 958-965; Kayagaki, N., et al., (2002) Immunity 10, 515-524). The extracellular domain (ECD) of BR3 is also divergent. TNFR family members are usually characterized by the presence of multiple cysteine-rich domains (CRDs) in their extracellular region; each CRD is typically composed of ˜40 residues stabilized by six cysteines in three disulfide bonds. Conventional members of this family make contacts with ligand through two CRDs interacting with two distinct patches on the ligand surface (reviewed in Bodmer, J.-L., et al., (2002) Trends Biochem. Sci. 27, 19-26). However, the BR3 ECD (SEQ ID NO: 60) contains only four cysteine residues, capable of forming a partial CRD at most, raising the question of how such a small receptor imparts high-affinity ligand binding.

The partial CRD of BR3 has a cysteine spacing distinct from other modules described previously. A core region of only 19 residues adopts a stable structure in solution. The BR3 fold is analogous to the first half of a canonical TNFR CRD but is stabilized by an additional noncanonical disulfide bond. Several BLyS-binding determinants have been identified by shotgun alanine-scanning mutagenesis of the BR3 ECD (SEQ ID NO: 60) expressed on phage. Several of the key BLyS-binding residues are presented from a beta-turn that we have shown previously to be sufficient for ligand binding when transferred to a structured beta-hairpin scaffold [Kayagaki, N., et al., (2002) Immunity 17, 515-524]. Outside of the turn, mutagenesis identified additional hydrophobic contacts that enhance the BLyS-BR3 interaction. The crystal structure of the minimal hairpin peptide, bhpBR3, in complex with BLyS revealed intimate packing of the six-residue BR3 turn into a cavity on the ligand surface. Thus, BR3 binds BLyS through a highly focused interaction site, unprecedented in the TNFR family.

Previously it has been shown that the BLyS-binding domain of BR3 resides within a 26-residue core region (Kayagaki, et al., supra). Six BR3 residues, when structured within a hairpin peptide (bhpBR3), were sufficient to confer BLyS binding and block BR3-mediated signaling. Others have reported polypeptides that have been purported to interact with BLyS (e.g., WO 02/24909, WO 03/035846, WO 02/16312, WO02/02641). However, despite these reports, there is a need for alternative and/or better peptide molecules to inhibit BLyS activity for research and medicinal purposes, including treating and diagnosing diseases using those BLyS binding polypeptides and developing small molecule inhibitors of the BLyS signaling pathway. Thus, these are objects of this invention. It is also an object of this invention to develop, interalia, small peptides that can be easily synthesized by non-cellular methods, polypeptides with significant BLyS binding affinity, and polypeptides that have good stability.

### SUMMARY OF THE INVENTION

The present invention relates to a polypeptide comprising the sequence of Formula I:
X₁-C_{N}-X₃-D-X₅-L-X₇-X₈-X₉-X₁₀-X₁₁-X₁₂-C_{T}-X₁₄-X₁₅-X₁₆-X₁₇ (Formula I)
(SEQ ID NO:1)

wherein X₁, X₃, X₅, X₇, X₈, X₉, X₁₀, X₁₁, X₁₂, X₁₄, X₁₅ and X₁₇ are any amino acid except cysteine; and

wherein X₁₆ is an amino acid selected from the group consisting of L, F, I and V; and

wherein the polypeptide does not comprise a cysteine within seven amino acid residues N-terminal to C_{N} (cysteine N terminal) and C-terminal to C_{T} (cysteine C terminal) of Formula I.

In some embodiments, a polypeptide comprising the sequence of Formula I has C_{N} and C_{T} joined by disulfide bonding; X₅LX₇X₈ forming the conformation of a type I beta turn structure with the center of the turn between L and X₇; and has a positive value for the dihedral angle phi of X₈. See FIG. 13.

In some embodiments, X₁₀ is selected from the group consisting of W, F, V, L, I, Y, M and a non-polar amino acid. (SEQ ID NO:2). In some embodiments, X₁₀ is W. (SEQ ID NO:3). In some embodiments, X₃ is an amino acid selected from the group consisting of M, V, L, I, Y, F, W and a non-polar amino acid. (SEQ ID NO:4). In some embodiments, X₅ is selected from the group consisting of V, L, P, S, I, A and R. (SEQ ID NO:5). In some embodiments, X₇ is selected from the group consisting of V, T, I and L. (SEQ ID NO:6). In some embodiments, X₇ is not T or I. (SEQ ID NO:7).

In some embodiments, X₈ is selected from the group consisting of R, K, G, N, H and all D-amino acids. (SEQ ID NO:8). In some embodiments, X₉ is selected from the group consisting of H, K, A, R and Q. (SEQ ID NO:9). In some embodiments, X₁₁ is I or V. (SEQ ID NO:10). In some embodiments, X₁₂ is selected from the group consisting of P, A, D, E and S. (SEQ ID NO:11). In some embodiments, X₁₆ is L. (SEQ ID NO:12). In specific embodiments, the sequence of Formula I is a sequence selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17).

The present invention also relates to a polypeptide comprising the sequence of Formula II:
X₁-C_{N}-X₃-D-X₅-L-V-X₈-X₉-W-X₁₁-X₁₂-CT-X₁₄-X₁₅-L-X₁₇ (Formula II) (SEQ ID NO:18)

wherein X₁, X₃, X₅, X₈, X₉, X₁₁, X₁₂, X₁₄, X₁₅ and X₁₇ are any amino acid, except cysteine;

wherein the polypeptide does not comprise a cysteine within seven amino acid residues N-terminal to C_{N} (cysteine N terminal) and C-terminal to C_{T} (cysteine C terminal) of Formula II; and

wherein a disulfide bond is formed between C_{N} and C_{T}.

In some embodiments, a polypeptide comprising the sequence of Formula II has the conformation of X₅LVX₈ forming a type I beta turn structure with the center of the turn between L and V; and has a positive value for the dihedral angle phi of X₈.

In some embodiments, X₁, X₃, X₅, X₈, X₉, X₁₁, X₁₂, X₁₄, X₁₅ and X₁₇ are selected from a group of amino acids consisting of L, P, H, R, I, T, N, S, V, A, D, and G. (SEQ ID NO:19).

In some embodiments of Formula II, X₃ is an amino acid selected from the group consisting of Norleucine, M, V, L, I, Y, F, W and a non-polar amino acid. (SEQ ID NO:20). In some embodiments of Formula II, X₅ is selected from the group consisting of V, L, P, S, I, A and R. (SEQ ID NO:21). In some embodiments of Formula II, X₈ is selected from the group consisting of R, K, G, N, H and all D-amino acids. (SEQ ID NO:22). In some embodiments of Formula II, X₉ is selected from the group consisting of H, K, A, R and Q. (SEQ ID NO:23). In some embodiments, X₁₁ is selected from the group consisting of I and V. (SEQ ID NO:24). In some embodiments, X₁₂ is selected from the group consisting of P, A, D, E, and S. (SEQ ID NO:25).

The present invention also relates to a polypeptide comprising the sequence of Formula III:
E-C_{N}-F-D-X₅-L-V-X₈-X₉-W-V-X₁₂-C_{T}-X₁₄-X₁₅-X₁₆-X₁₇ (Formula III) (SEQ ID NO:26)

wherein X₅, X₈, X₉, X₁₂, X₁₄, X₁₅ and X₁₇ are any amino acid except cysteine;

wherein X₁₆ is an amino acid selected from the group consisting of L, F, I and V;

wherein the polypeptide does not comprise a cysteine within seven amino acid residues N-terminal to C_{N} (cysteine N terminal) and C-terminal to C_{T} (cysteine C terminal) of Formula III; and

wherein C_{N} and C_{T} are joined by disulfide bonding.

In some embodiments of Formula III, a polypeptide comprising the contiguous sequence of Formula III has a disulfide bond between C_{N} and C_{T} and forms a type I beta turn structure with the center of the turn between L and V at X₅LVX₈; and has a positive value for the dihedral angle phi of X₈.

In some embodiments of Formula III, X₅, X₈, X₉, X₁₂, X₁₄, X₁₅ and X₁₇ are selected from the group consisting of L, P, H, R, I, T, N, S, V, A, D, and G. (SEQ ID NO:27). In some embodiments of Formula III, X₅ is L and X₈ is R. (SEQ ID NO:28). In some embodiments of Formula III, X₉ is selected from the group consisting of H, K, A, S, R and Q. (SEQ ID NO:29). In some embodiments of Formula III, X₁₂ is selected from the group consisting of P, A, D, E and S. (SEQ ID NO:30). In some embodiments of Formula III, X₁₂ is P. (SEQ ID NO:31). In some embodiments of Formula III, X₁₆ is L. (SEQ ID NO:32).

In specific embodiments, the sequence of Formula III is selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17).

The present invention also relates to a contiguous polypeptide sequence selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17).

The present invention also relates to a polypeptide comprising at least 88% sequence identity with a contiguous polypeptide sequence selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17).

The present invention also relates to a polypeptide comprising a sequence selected from any one of the sequences described in FIG. 12A-C. Polypeptides comprising any one of the sequences described in FIG. 12A-C, wherein the cysteines of the sequence are joined by disulfide bonding, wherein the sequence between the fifth and eighth residues of the sequence forms a conformation of a type I beta turn structure with the center of the turn between L and X₇ and the eighth residue has a positive value for the dihedral angle phi are contemplated.

In some embodiments, the polypeptides of this invention comprise sequences N-terminal, C-terminal or both N-terminal and C-terminal to the sequence of Formula I (SEQ ID NO:1) or Formula II (SEQ ID NO:18) or Formula III (SEQ ID NO:26) that are heterologous to a native sequence BR3 polypeptide. According to some embodiments, a BLyS binding sequence selected from the group consisting of: Formula I (SEQ ID NO:1), Formula II (SEQ ID NO:18), Formula III (SEQ ID NO:26), FIG. 12A-C or ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16) or ECFDLLVRHWVACGLLR (SEQ ID NO:17) is fused or conjugated to an immunoadhesion protein. In some embodiments, the BLyS binding sequence is fused or conjugated to an antibody. In a further embodiment, the antibody is selected from the group consisting of a F(ab) antibody, F(ab′)₂ antibody and a scFv antibody. In alternative or additional embodiments, the antibody is selected from the group consisting of a humanized antibody and a multi-specific antibody.

According to some embodiments, a polypeptide of this invention is conjugated to or used in combination with an agent selected from the group consisting of a growth inhibitory agent, a cytotoxic agent, a detection agent, an agent that improves the bioavailability of the polypeptide and an agent that improves the half-life of the polypeptide. In a further embodiment of this invention, the cytotoxic agent is selected from the group consisting of a toxin, an antibiotic and a radioactive isotope. According to some embodiments of this invention, the polypeptide is less than 50 amino acids in length, less than 25 amino acids in length, or is 17 amino acids in length (17-mer).

Another aspect of the invention involves polypeptides that comprise at least one and more preferably, more than one of a polypeptide comprising a sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C. The polypeptides that are linked together can have the same sequence or have different sequences. In some embodiments, these polypeptides can be joined to one another, optionally, through the use of a linker.

A polypeptide of this invention is produced in bacteria in some embodiments. In some embodiments, a polypeptide of this invention is produced in CHO cells. The present invention also relates to a nucleic acid molecule encoding the polypeptide of this invention. The present invention also relates to a vector comprising the nucleic acid molecule of this invention. A vector of this invention is useful, e.g., for expressing the polypeptide for production of purified protein and/or gene therapy. The present invention relates to a host cell comprising the nucleic acid molecule or vector of this invention. The present invention relates to a method for producing a polypeptide comprising culturing a host cell comprising a vector or nucleic acid molecule of this invention under conditions suitable for expressing the polypeptide from the vector.

The present invention relates to compositions comprising a polypeptide of this invention, and optionally further comprising a physiologically acceptable carrier. In some embodiments, the compositions of this invention further comprise an additional therapeutic agent. According to some embodiments, the additional therapeutic agent is a drug for treating a disease selected from the group consisting of an immune-related disease and a cancer. According to some embodiments, the additional therapeutic agent is a drug that is used to treat the symptoms of a disease selected from the group consisting of an immune-related disease and a cancer. According to some embodiments, the drug is an anti-CD20 antibody such as the RITUXAN® antibody.

The present invention relates to a method for selecting a BLyS antagonist comprising identifying a molecule that inhibits BLyS from binding to a polypeptide according to this invention. According to some embodiments, the molecule is a small molecule.

The present invention relates to a method for inhibiting BLyS binding to BR3 comprising contacting BLyS to a polypeptide of this invention. The present invention also relates to a method for inhibiting BLyS binding to BR3 in a mammal comprising administering a polypeptide of this invention to the animal. The present invention also relates to a method for inhibiting BLyS signaling in a mammal comprising administering a polypeptide of this invention in an amount effective to reduce the number of B cells in the mammal.

The present invention also relates to a method for making an antibody comprising immunizing an animal with a polypeptide of this invention.

The present invention relates to a method for preventing or treating an, immune-related condition in a mammal in need of treatment therefor comprising treating the mammal with a therapeutically effective amount of a composition according to this invention. In some embodiments, the immune related disease is selected from the group consisting of rheumatoid arthritis, multiple sclerosis and systemic lupus erythematosis.

The present invention relates to a method for preventing or treating a cancer in a mammal in need of treatment therefor comprising treating the mammal with a therapeutically effective amount of a composition of this invention. In some embodiments, the cancer is selected from the group consisting of leukemia, lymphoma, or myeloma. In some embodiments, the method further comprises administering a therapeutically effective amount of an anti-CD20 antibody to the mammal. In specific embodiments, the anti-CD20 antibody is the RITUXAN® antibody.

The present invention relates to a method for preventing or treating a cancer in a mammal in need of treatment therefor comprising treating the mammal with a therapeutically effective amount of a composition of this invention. In some embodiments, the cancer is selected from the group consisting of leukemia, lymphoma, or myeloma. In some embodiments, the method further comprises administering a therapeutically effective amount of a CD20 binding antibody to the mammal. In specific embodiments, the anti-CD20 antibody is the RITUXAN® antibody. The present invention also relates to a method of depleting B cells from a mixed population of cells comprising contacting the mixed population of cells with a BLyS antagonist and a CD20 binding antibody.

The present invention relates to methods of diagnosing the levels of BLyS in a patient comprising the steps of contacting a polypeptide of this invention to the BLyS of the patient and evaluating the amount of BLyS bound to the polypeptide.

The present invention relates to conjugates of a polypeptide of this invention to a non-proteinaceous polymer. In some embodiments, the nonproteinaceous polymer is a hydrophilic, synthetic polymer, such as polyethylene glycol (PEG). In some embodiments, the non-proteinaceous polymer is selected from the group consisting of 2 k PEG, 5 k PEG and 20 k PEG.

### BRIEF DESCRIPTION OF THE FIGURES

FIG. 1 shows a polynucleotide sequence encoding a native sequence of human BLyS (SEQ ID NO:33) and its amino acid sequence (SEQ ID NO:34).

FIG. 2A shows a polynucleotide sequence (start and stop codons are underlined) encoding a native sequence of human BR3 (SEQ ID NO:35), and FIG. 2B shows its amino acid sequence (SEQ ID NO:36)

FIG. 3 shows a polynucleotide sequence (start and stop codons are underlined) encoding murine BR3 (SEQ ID NO:37), and

FIG. 4 shows a sequence alignment of human (SEQ ID NO:34) and murine BR3 (SEQ ID NO:38), with identical amino acids indicated by letter and conserved amino acids indicated by a plus sign below.

FIG. 5 shows the cDNA nucleotide sequence for human CD20 (SEQ ID NO:39).

FIG. 6 shows the amino acid sequence of human CD20 showing predicted transmembrane (boxed) and extracellular (underlined) regions. (SEQ ID NO:40),

FIG. 7 is a sequence alignment comparing the amino acid sequences of the light chain variable domain (V_{L}) of murine 2H7 (SEQ ID NO:41), humanized 2H7 v16 variant (SEQ ID NO:42), and human kappa light chain subgroup I (SEQ ID NO:43).

FIG. 8 is a sequence alignment which compares the heavy chain variable domain (V_{H}) of murine 2H7 (SEQ ID NO:47), humanized 2H7 v16 variant (SEQ ID NO:48), and the human consensus sequence of heavy chain subgroup III (SEQ ID NO:49).

FIG. 9 shows the phage display 17mer library design where positions indicated by an “X” were randomized in each library using the degenerate codons NNS codon (library 1) or VNC (library 2).

FIG. 10 is an overview of phage selection.

FIG. 11 shows phage ELISA data in the absence or presence of 50 nM BLyS where inhibition is calculated as a percent reduction in signal of the 50 nM BLyS containing wells relative to the reference wells with background subtracted from each.

FIG. 12A-C shows the amino acid sequence of 17mers selected from the phage display libraries for high affinity BLyS binding.

FIG. 13 is a stereoview model of the three-dimensional structure of a peptide of this invention.

FIG. 14A-C shows DNA sequence of 17mers selected from the phage display libraries for high affinity BLyS binding. Bases from the leader and linker sequence (12 each) flank the region corresponding to 17mer sequence FIG. 15 presents ELISA competition data of BLyS for 17mers displayed on phage with IC50 values range from 0.4 nM (clone 44) to 11 nM (clone22).

FIG. 16 shows a competitive displacement of biotinylated mini-BR3 (SEQ ID NO: 59) measured by ELISA for BR3 extracellular domain (SEQ ID NO: 60)(open circles and open squares), BLyS0027 (SEQ ID NO:17) (diamond and “x”), BLyS0048 (SEQ ID NO:14) (“+” and triangles) and BLyS0051 (SEQ ID NO:13) (closed circles and closed squares).

FIG. 17A-B shows HPLC chromatograms of PEG-polypeptide conjugates.

FIG. 18 presents ELISA competition data of BLyS for 17mers and 17mer-PEG conjugates.

FIG. 19 presents ELISA competition data of BLyS for a 17mer and a 17mer-20 kPEG conjugate.

### DETAILED DESCRIPTION

A polypeptide of the present invention includes antibodies, immunoadhesins, peptide fusions and conjugates comprising the sequences disclosed herein. The polypeptides of the present invention, alone or in combination with other proteins bind native sequence BLyS. According to some embodiments, the polypeptide is a BLyS antagonist. According to some embodiments, a polypeptide of the present invention can be modified by conjugation to a label (a detectable compound or composition or an agent that promotes detection), a therapeutic agent, a protecting group, and an agent that promotes the bioavailability or half-life of the polypeptide. Polypeptides comprising a hairpin loop structure in the sequences disclosed herein are contemplated.

Definitions

The terms “BLyS,” “BLyS polypeptide,” “TALL-1” or “TALL-1 polypeptide,” “BAFF” when used herein encompass “native sequence BLyS polypeptides” and “BLyS variants”. “BLyS” is a designation given to those polypeptides which are encoded by any one of the amino acid sequences shown below:

| (SEQ ID NO:34) | |
|---|---|
| Human BLyS sequence | |
| 1 | MDDSTEREQS RLTSCLKKRE EMKLKECVSI LPRKESPSVR SSKDGKLLAA TLLLALLSCC |
| 61 | LTVVSFYQVA ALQGDLASLR AELQGHHAEK LPAGAGAPKA GLEEAPAVTA GLKIFEPPAP |
| 121 | GEGNSSQNSR NKRAVQGPEE TVTQDCLQLI ADSETPTIQK GSYTFVPWLL SFKRGSALEE |
| 181 | KENKILVKET GYFFIYGQVL YTDKTYAMGH LIQRKKVHVF GDELSLVTLF RCIQNMPETL |
| 241 | PNNSCYSAGI AKLEEGDELQ LAIPRENAQI SLDGDVTFFG ALKLL |

(SEQ ID NO:53)
Mouse BLyS sequence

| | |
|---|---|
| 1 | MDESAKTLPP PCLCFCSEKG EDMKVGYDPI TPQKEEGAWF GICRDGRLLA ATLLLALLSS |
| 61 | SFTAMSLYQL AALQADLMNL RMELQSYRGS ATPAAAGAPE LTAGVKLLTP AAPRPHNSSR |
| 121 | GHRNRRAFQG PEETEQDVDL SAPPAPCLPG CRHSQHDDNG MNLRNIIQDC LQLIADSDTP |
| 181 | TIRKGTYTFV PWLLSFKRGN ALEEKENKIV VRQTGYFFIY SQVLYTDPIF AMGHVIQRKK |
| 241 | VHVFGDELSL VTLFRCIQNM PKTLPNNSCY SAGIARLEEG DEIQLAIPRE NAQISRNGDD |
| 301 | TFFGALKLL |

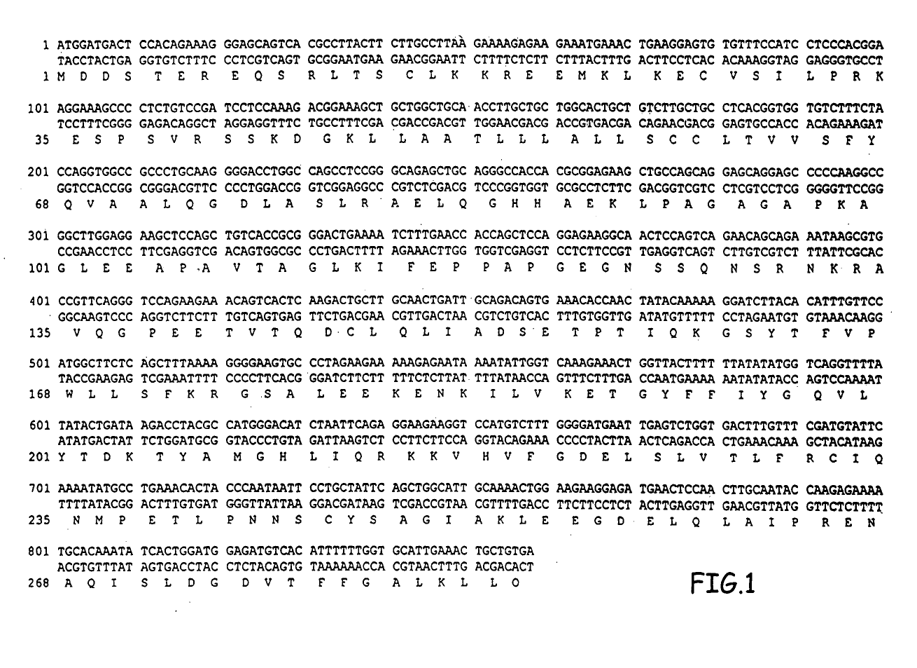

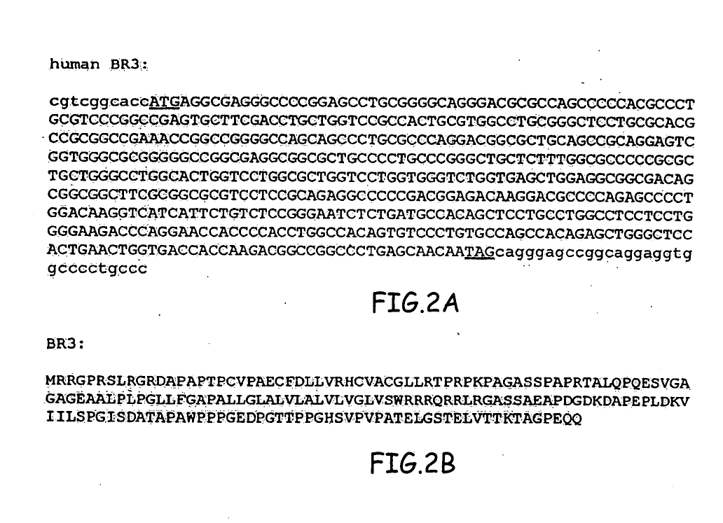

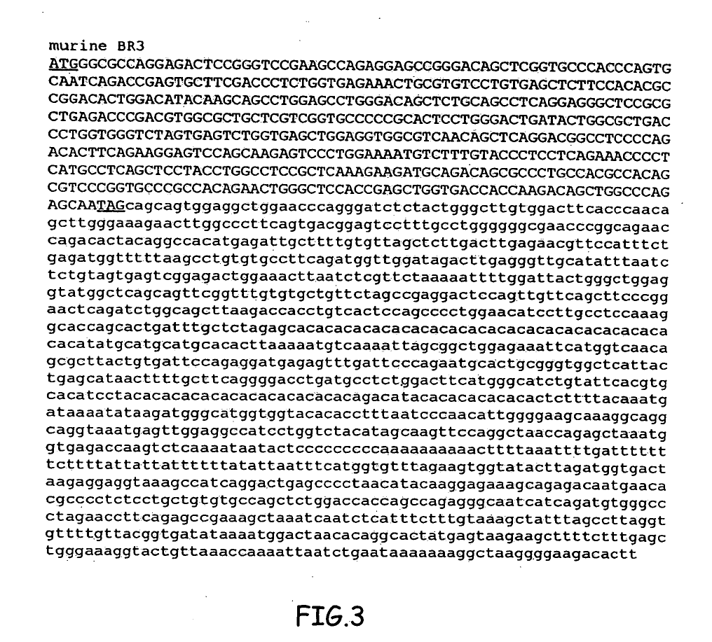

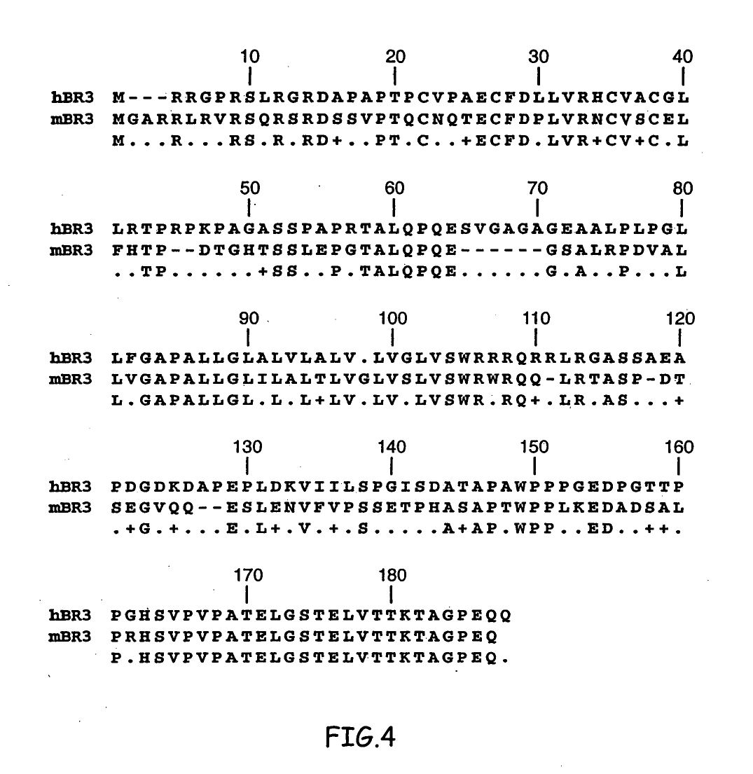

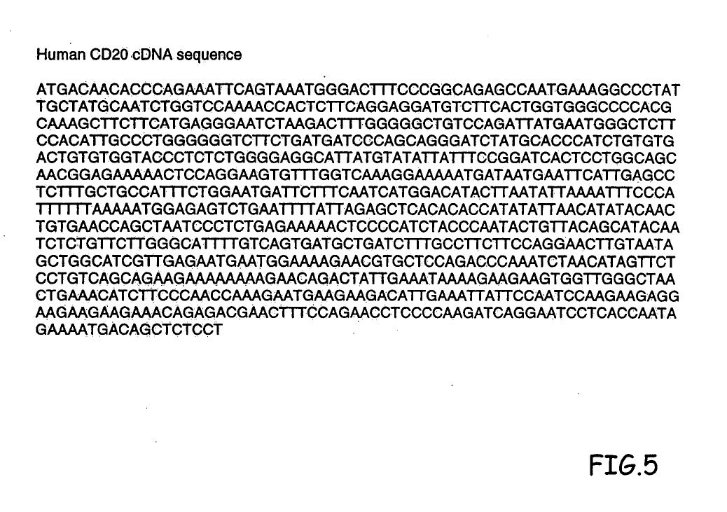

and FIG. 1 and homologs and fragments and variants thereof, which have a biological activity of the native sequence BLyS. A biological activity of BLyS can be selected from the group consisting of promoting B cell survival, promoting B cell maturation and binding to BR3. Variants of BLyS will preferably have at least 80% or any successive integer up to 100% including, more preferably, at least 90%, and even more preferably, at least 95% amino acid sequence identity with a native sequence of a BLyS polypeptide. A “native sequence” BLyS polypeptide comprises a polypeptide having the same amino acid sequence as the corresponding BLyS polypeptide derived from nature. For example, BLyS, exists in a soluble form following cleavage from the cell surface by furin-type proteases. Such native sequence BLyS polypeptides can be isolated from nature or can be produced by recombinant and/or synthetic means.

The term “native sequence BLyS polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “BLyS” includes those polypeptides described in Shu et al., J. Leukocyte Biol., 65:680 (1999); GenBank Accession No. AF136293; WO98/18921 published May 7, 1998; EP 869,180 published Oct. 7, 1998; WO98/27114 published Jun. 25, 1998; WO99/12964 published Mar. 18, 1999; WO99/33980 published Jul. 8, 1999; Moore et al., Science, 285:260-263 (1999); Schneider et al., J. Exp. Med., 189:1747-1756 (1999); Mukhopadhyay et al., J. Biol. Chem., 274:15978-15981 (1999).

The term “BLyS antagonist” as used herein is used in the broadest sense, and includes any molecule that (1) binds a native sequence BLyS polypeptide or binds a native sequence BR3 polypeptide to partially or fully block BR3 interaction with BLyS polypeptide, and (2) partially or fully blocks, inhibits, or neutralizes native sequence BLyS signaling. Native sequence BLyS polypeptide signaling promotes, among other things, B cell survival and B cell maturation. The inhibition, blockage or neutralization of BLyS signaling results in, among other things, the reduction in number of B cells. A BLyS antagonist according to this invention will partially or fully block, inhibit, or neutralize one or more biological activities of a BLyS polypeptide, in vitro or in vivo. In some embodiments, a biologically active BLyS potentiates any one or combination of the following events in vitro or in vivo: an increased survival of B cells, an increased level of IgG and/or IgM, an increased numbers of plasma cells, and processing of NF-κb2/p100 to p52 NF-κb in splenic B cells (e.g., Batten, M et al., (2000) J. Exp. Med. 192:1453-1465; Moore, et al., (1999) Science 285:260-263; Kayagaki, et al., (2002) 10:515-524). Several assays useful for testing BLyS antagonists according to this invention are described herein.

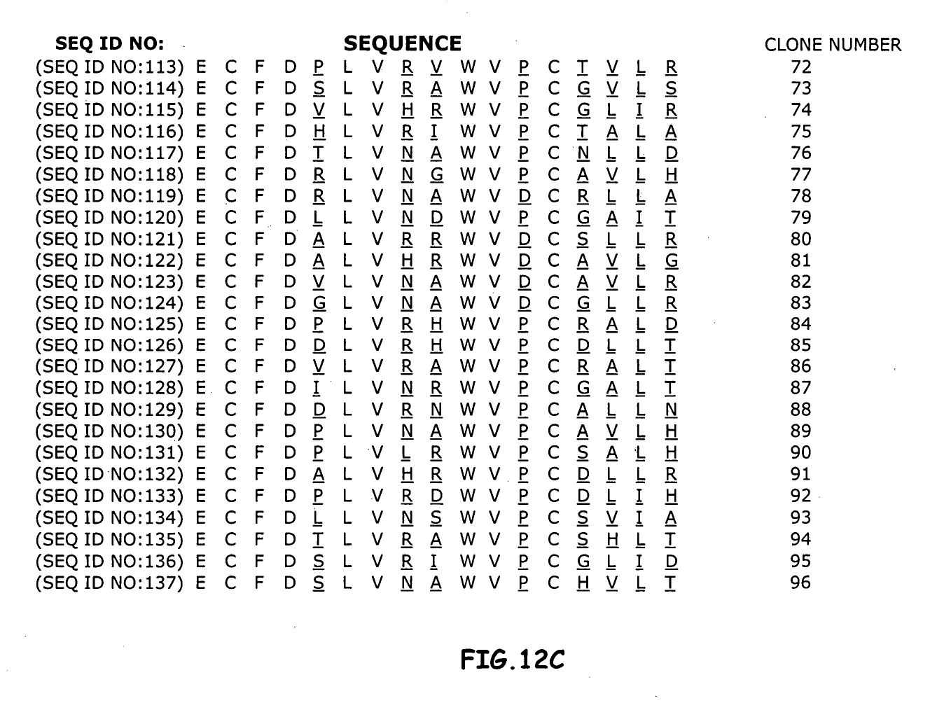

As mentioned above, a BLyS antagonist can function in a direct or indirect manner to partially or fully block, inhibit or neutralize BLyS signaling, in vitro or in vivo. For instance, the BLyS antagonist can directly bind BLyS. For example, anti-BLyS antibodies that bind within a region of human BLyS comprising residues 162-275 and/or a neighboring residue of a residue selected from the group consisting of 162, 163, 206, 211, 231, 233, 264 and 265 of human BLyS such that the antibody sterically hinders BLyS binding to BR3 is contemplated. In another example, a direct binder is a polypeptide comprising the extracellular domain of a BLyS receptor such as TACI, BR3 and BCMA. In another example, BLyS antagonists include the polypeptides having a sequence of that of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C, as described herein.

In some embodiments, a BLyS antagonist according to this invention includes anti-BLyS antibodies, immunoadhesins and small molecules. In a further embodiment, the immunoadhesin comprises a BLYS binding region of a BLyS receptor (e.g., an extracellular domain of BR3, BCMA or TACI). In a still further embodiment, the immunoadhesin is BR3-Fc or polypeptides having a sequence of that of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C, optionally, fused or conjugated to an Fc portion of an immunoglobulin.

According to some embodiments, the BLyS antagonist binds to a BLyS polypeptide with a binding affinity of 100 nM or less. According to other embodiments, the BLyS antagonist binds to a BLyS polypeptide with a binding affinity of 10 nM or less. According to yet other embodiment, the BLyS antagonist binds to a BLyS polypeptide with a binding affinity of 1 nM or less.

The terms “BR3”, “BR3 polypeptide” or “BR3 receptor” when used herein encompass “native sequence BR3 polypeptides”. “BR3” is a designation given to those polypeptides comprising any one of the following polynucleotide sequences and homologs thereof:

| (SEQ ID NO:36) | |
|---|---|
| (a) human BR3 sequence | |
| 1 | MRRGPRSLRG RDAPAPTPCV PAECFDLLVR HCVACGLLRT PRPKPAGASS PAPRTALQPQ |
| 61 | ESVGAGAGEA ALPLPGLLFG APALLGLALV LALVLVGLVS WRRRQRRLRG ASSAEAPDGD |
| 121 | KDAPEPLDKV IILSPGISDA TAPAWPPPGE DPGTTPPGHS VPVPATELGS TELVTTKTAG |
| 181 | PEQQ |
| (SEQ ID NO:54) | |
| (b) alternative human BR3 sequence | |
| 1 | MRRGPRSLRG RDAPAPTPCV PAECFDLLVR HCVACGLLRT PRPKPAGAAS SPAPRTALQP |
| 61 | QESVGAGAGE AALPLPGLLF GAPALLGLAL VLALVLVGLV SWRRRQRRLR GASSAEAPDG |
| 121 | DKDAPEPLDK VIILSPGISD ATAPAWPPPG EDPGTTPPGH SVPVPATELG STELVTTKTA |
| 181 | GPEQQ |
| (SEQ ID NO:38) | |
| (c) murine BR3 sequence | |
| 1 | MGARRLRVRS QRSRDSSVPT QCNQTECFDP LVRNCVSCEL FHTPDTGHTS SLEPGTALQP |
| 61 | QEGSALRPDV ALLVGAPALL GLILALTLVG LVSLVSWRWR QQLRTASPDT SEGVQQESLE |
| 121 | NVFVPSSETP HASAPTWPPL KEDADSALPR HSVPVPATEL GSTELVTTKT AGPEQ |
| (SEQ ID NO:55) | |
| (d) rat BR3 sequence | |
| 1 | MGVRRLRVRS RRSRDSPVST QCNQTECFDP LVRNCVSCEL FYTPETRHAS SLEPGTALQP |
| 61 | QEGSGLRPDV ALLFGAPALL GLVLALTLVG LVSLVGWRWR QQRRTASLDT SEGVQQESLE |
| 121 | NVFVPPSETL HASAPNWPPF KEDADNILSC HSIPVPATEL GSTELVTTKT AGPEQ |

A “native sequence” BR3 polypeptide comprises a polypeptide having the same amino acid sequence as the corresponding BR3 polypeptide derived from nature. Such native sequence BR3 polypeptides can be isolated from nature or can be produced by recombinant and/or synthetic means. The term “native sequence BR3 polypeptide” specifically encompasses naturally-occurring truncated, soluble or secreted forms (e.g., an extracellular domain sequence), naturally-occurring variant forms (e.g., alternatively spliced forms) and naturally-occurring allelic variants of the polypeptide.

A BR3 “extracellular domain” or “ECD” refers to a form of the BR3 polypeptide that is essentially free of the transmembrane and cytoplasmic domains. ECD forms of BR3 include those comprising any one of amino acids 1 to 77, 2 to 62, 2-71, 1-61 and 2-63 of BR3. BR3 ECD comprising amino acids 1-61 is presented in SEQ ID NO:60.

Mini-BR3 is a 26-residue core region of the BLyS-binding domain of BR3. Mini-BR3 (SEQ. ID:59): TPCVPAECFD LLVRHCVACG LLRTPR The term “amino acid” is used in its broadest sense and is meant to include the naturally occurring L α-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes all D-amino acids as well as chemically modified amino acids such as amino acid analogs, naturally occurring amino acids that are not usually incorporated into proteins such as Norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio (The Peptides: Analysis, Synthesis, Biology,) Eds. Gross and Meiehofer, Vol. 5 p 341, Academic Press, Inc, N.Y. 1983, which is incorporated herein by reference.

Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (O)

(3) acidic: Asp (D), Glu (E)

(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

The term “conservative” amino acid substitution as used within this invention is meant to refer to amino acid substitutions that substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. In general, substitutions within a group may be considered conservative with respect to structure and function. However, the skilled artisan will recognize that the role of a particular residue is determined by its context within the three-dimensional structure of the molecule in which it occurs. For example, Cys residues may occur in the oxidized (disulfide) form, which is less polar than the reduced (thiol) form. The long aliphatic portion of the Arg side chain may constitute a critical feature of its structural or functional role, and this may be best conserved by substitution of a nonpolar, rather than another basic residue. Also, it will be recognized that side chains containing aromatic groups (Trp, Tyr, and Phe) can participate in ionic-aromatic or “cation-pi” interactions. In these cases, substitution of one of these side chains with a member of the acidic or uncharged polar group may be conservative with respect to structure and function. Residues such as Pro, Gly, and Cys (disulfide form) can have direct effects on the main chain conformation, and often may not be substituted without structural distortions.

Substantial modifications in function or immunological identity of a protein are accomplished by selecting substitutions that differ significantly in their effect on maintaining (a) the structure of the polypeptide backbone in the area of the substitution, for example as a sheet or in helical conformation; (b) the charge or hydrophobicity of the molecule at the target site; or (c) the bulk of the side chain. Non-conservative amino acid substitutions refer to amino acid substitutions that substitute functionally non-equivalent amino acids, for example, by exchanging a member of one group of amino acids described above for a member of another group.

A useful method for identification of certain residues or regions in a protein that are preferred locations for mutagenesis is called “alanine scanning mutagenesis” as described by Cunningham and Wells Science, 244:1081-1085 (1989). A residue or group of target residues are identified (e.g., charged residues such as Arg, Asp, His, Lys, and Glu) and replaced by a neutral or negatively charged amino acid (most preferably alanine or polyalanine) to affect the interaction of the amino acids with a binding target. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed variants are screened for the desired activity.

The term, “dihedral angle” refers to a rotation about a bond. See e.g., Creighton, T. E., (1993) Protein: Structures and Molecular Properties, 2 ed., W. H. Freeman and Company, New York, N.Y.

The term, “phi,” is a dihedral angle that denotes a rotation about the N-C^{α} bond of an amino acid. See e.g., Creighton, T. E., (1993) Protein:Structures and Molecular Properties, 2 ed., W. H. Freeman and Company, New York, N.Y. All D amino acids and glycine will readily adopt a backbone conformation having a positive phi angle. Typically, the remaining L amino acids prefer conformations with a negative phi angle and will only readily adopt a positive phi angle if placed in a three-dimensional environment (tertiary) structure that supports such a backbone conformation.

Type I beta turns are described in Hutchinson, E. G. & Thornton, J. M. (1994) A revised set of potentials for beta turn formation in proteins. Protein Science 3, 2207-2216.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

A “conjugate” refers to any hybrid molecule, including fusion proteins and as well as molecules that have both amino acid or protein portions and non-protein portions. Conjugates may be synthesized by a variety of techniques known in the art including, for example, recombinant DNA techniques, solid phase synthesis, solution phase synthesis, organic chemical synthetic techniques or a combination of these techniques. The choice of synthesis will depend upon the particular molecule to be generated. For example, a hybrid molecule not entirely “protein” in nature may be synthesized by a combination of recombinant techniques and solution phase techniques.

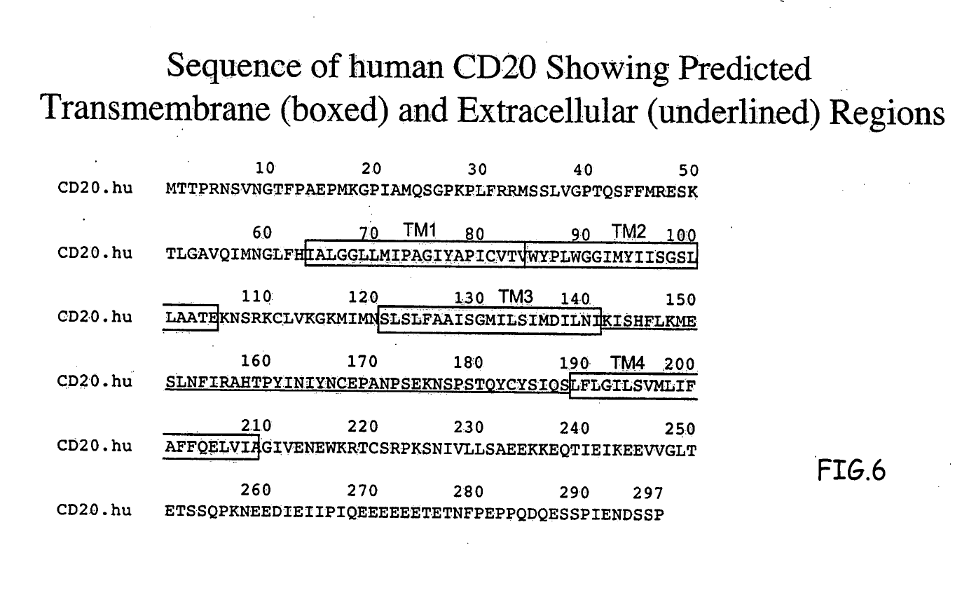

The “CD20 antigen” is a non-glycosylated, transmembrane, phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19): 11282-11287 (1989). The cDNA sequence for of human CD20 is presented in FIG. 5. The amino acid sequence is shown in FIG. 6 with predicted transmembrane regions enclosed in boxes and extracellular regions underlined. Putative Domains are 1-63: Cytoplasmic; 64-84: Transmembrane; 85-105: Transmembrane; 106-120: Cytoplasmic; 121-141: Transmembrane; 142-188: Extracellular; 189-209: Transmembrane; 210-297: Cytoplasmic; 81-167: Disulfide bond.

“CD20 binding antibody” and “anti-CD20 antibody” are used interchangeably herein and encompass all antibodies that bind CD20 with sufficient affinity such that the antibody is useful as a therapeutic agent in targeting a cell expressing the antigen, and do not significantly cross-react with other proteins such as a negative control protein in the assays described below. Bispecific antibodies wherein one arm of the antibody binds CD20 are also contemplated. Also encompassed by this definition of CD20 binding antibody are functional fragments of the preceding antibodies. The CD20 binding antibody will bind CD20 with a Kd of <10 nM. In preferred embodiments, the binding is at a Kd of <7.5 nM, more preferably <5 nM, even more preferably at between 1-5 nM, most preferably, <1 nM.

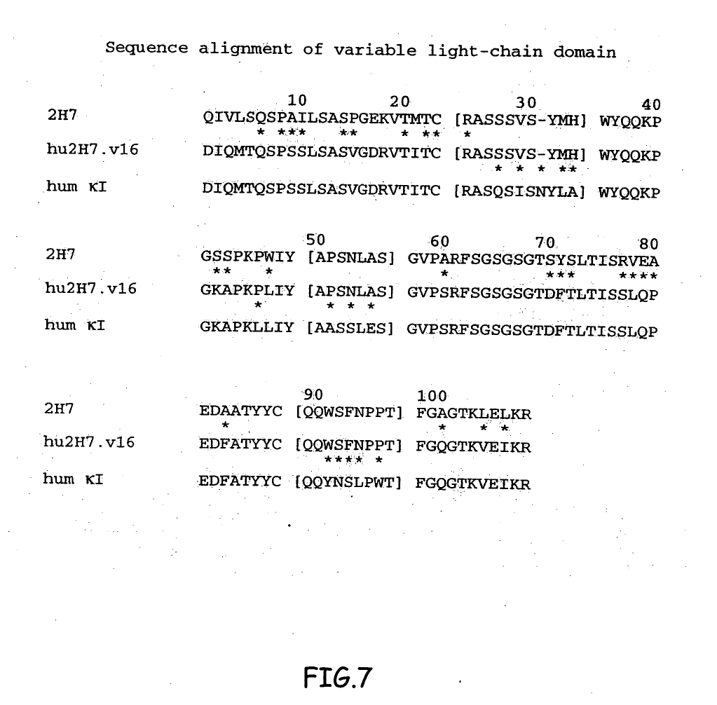

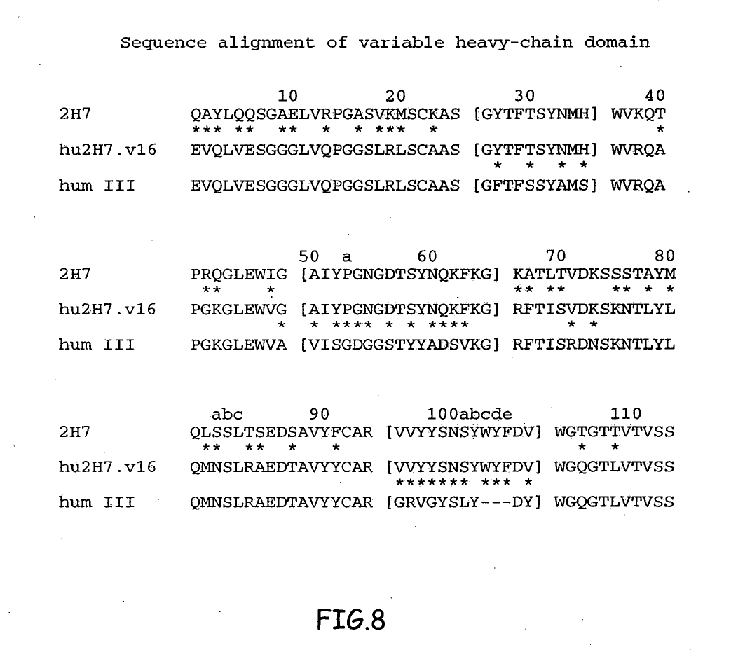

In a specific embodiment, the anti-CD20 antibodies bind human and primate CD20. In specific embodiments, the antibodies that bind CD20 are humanized or chimeric. CD20 binding antibodies include rituximab (RITUXAN®), m2H7 (murine 2H7), hu2H7 (humanized 2H7) and all its functional variants, including without limitation, hu2H7.v16 (v stands for version), v31, v73, v75, as well as fucose deficient variants. Sequence alignment of the variable region of the light chain domain for 2H7, hu2H7.v16 and hum κI is presented in FIG. 7. Sequence alignment of the variable region of the heavy chain domain for 2H7, hu2H7.v 16 and humIII is presented in FIG. 8. Sequences of some of the hu2H7 variant antibodies are also provided below:

(SEQ ID NO:56)
hu2H7.v16 L chain [232 aa]
MGWSCIILFLVATATGVHSDIQMTQSPSSLSASVGDRVTITCRASSSVSYMHWYQQKPGK
APKPLIYAPSNLASGVPSRFSGSGSGTDFTLTISSLQPEDFATYYCQQWSFNPPTFGQGT
KVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
(SEQ ID NO:57)
hu2H7.v16 H chain [471 aa]
MGWSCIILFLVATATGVHSEVQLVESGGGLVQPGGSLRLSCAASGYTFTSYNMHWVRQAP
GKGLEWVGAIYPGNGDTSYNQKFKGRFTISVDKSKNTLYLQMNSLRAEDTAVYYCARVVY
YSNSYWYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
(SEQ ID NO:58)
hu2H7.v31 H chain [471 aa]. The L chain is the same as that
of v16 above.
MGWSCIILFLVATATGVHSEVQLVESGGGLVQPGGSLRLSCAASGYTFTSYNMHWVRQAP
GKGLEWVGAIYPGNGDTSYNQKFKGRFTISVDKSKNTLYLQMNSLRAEDTAVYYCARVVY
YSNSYWYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKFN
WYVDGVEVHNAKTKPREEQYNATYRVVSVLTVLHQDWLNGKEYKCKVSNKALPAPIAATI
SKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK

Patents and patent publications concerning CD20 antibodies include U.S. Pat. Nos. 5,776,456, 5,736,137, 6,399,061, and 5,843,439, as well as US patent application nos. US 2002/0197255A1 and US 2003/0021781A1 (Anderson et al.); U.S. Pat. No. 6,455,043B1 and WO0/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US Application No. US2002/0006404 and WO02/04021 (Hanna and Hariharan); US Application No. US2002/0012665 A1 and WO01/74388 (Hanna, N.); US Application No. US2002/0009444A1, and WO01/80884 (Grillo-Lopez, A.); WO01/97858 (White, C.); US Application No. US2002/0128488A1 and WO02/34790 (Reff, M.); WO02/060955 (Braslawsky et al.); WO2/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); US Application No. US 2002/0004587A1 and WO01/77342 (Miller and Presta); US application no. US2002/0197256 (Grewal, I.); U.S. Pat. Nos. 6,090,365B1, 6,287,537B1, 6,015,542, 5,843,398, and 5,595,721, (Kaminski et al.); U.S. Pat. Nos. 5,500,362, 5,677,180, 5,721,108, and 6,120,767 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); WO00/74718 (Goldenberg and Hansen); WO00/76542 (Golay et al.); WO01/72333 (Wolin and Rosenblatt); U.S. Pat. No. 6,368,596B1 (Ghetie et al.); US Application No. US2002/0041847A1, (Goldenberg, D.); US Application No. US2003/0026801A1 (Weiner and Hartmann); WO02/102312 (Engleman, E.), each of which is expressly incorporated herein by reference. See, also, U.S. Pat. No. 5,849,898 and EP appln no. 330,191 (Seed et al.); U.S. Pat. No. 4,861,579 and EP332,865A2 (Meyer and Weiss); and WO95/03770 (Bhat et al.).

The CD20 antibodies can be naked antibody or conjugated to a cytotoxic compound such as a radioisotope, or a toxin. Such antibodies include the antibody ZEVALIN®, which is linked to the radioisotope, Yttrium-90 (IDEC Pharmaceuticals, San Diego, Calif.), and BEXXAR®, which is conjugated to 1-131 (Corixa, Wash.). The humanized 2H7 variants include those that have amino acid substitutions in the FR and affinity maturation variants with changes in the grafted CDRs. The substituted amino acids in the CDR or FR are not limited to those present in the donor or acceptor antibody. In other embodiments, the anti-CD20 antibodies of the invention further comprise changes in amino acid residues in the Fc region that lead to improved effector function including enhanced CDC and/or ADCC function and B-cell killing (also referred to herein as B-cell depletion). In particular, three mutations have been identified for improving CDC and ADCC activity: S298A/E333A/K334A (also referred to herein as a triple Ala mutant or variant; numbering in the Fc region is according to the EU numbering system; Kabat et al., supra) as described (Idusogie et al., supra (2001); Shields et al., supra).

Other anti-CD20 antibodies suitable for use with the present invention include those having specific changes that improve stability. In some embodiments, the chimeric anti-CD20 antibody has murine V regions and human C region. One such specific chimeric anti-CD20 antibody is RITUXAN® (RITUXIMAB®; Genentech, Inc.). Rituximab and hu2H7 can mediate lysis of B-cells through both complement-dependent cytotoxicity (CDC) and antibody-dependent cellular cytotoxicity (ADCC). Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

WO00/42072 (Presta) describes polypeptide variants with improved or diminished binding to FcRs. The content of that patent publication is specifically incorporated herein by reference. See, also, Shields et al. J. Biol. Chem. 9(2): 6591-6604 (2001).

The N-glycosylation site in IgG is at Asn297 in the CH2 domain. Additionally encompassed herein are humanized CD20-binding antibodies having a Fc region, wherein about 80-100% (and preferably about 90-99%) of the antibody in the composition comprises a mature core carbohydrate structure which lacks fucose, attached to the Fc region of the glycoprotein. Such antibodies show improvement in binding to FcγRIIIA (F158), which is not as effective as FcγRIIIA (V158) in interacting with human IgG.

“Functional fragments” of the CD20 binding antibodies of the invention are those fragments that retain binding to CD20 with substantially the same affinity as the intact full chain molecule from which they are derived and are able to deplete B cells as measured by in vitro or in vivo assays such as those described herein.

The term “antibody” is used in the broadest sense and specifically covers, for example, monoclonal antibodies, polyclonal antibodies, antibodies with polyepitopic specificity, single chain antibodies, and fragments of antibodies. According to some embodiments, a polypeptide sequences of this invention (e.g., a 17-mer) can be inserted into an antibody sequence, for example, inserted in the variable region or in a CDR such that the antibody can bind to and inhibit BLyS binding to BR3 or BLyS signaling. The antibodies comprising a polypeptide of this invention can be chimeric, humanized, or human. The antibodies comprising a polypeptide of this invention can be an antibody fragment. Such antibodies and methods of generating them are described in more detail below. Alternatively, an antibody of this invention can be produced by immunizing an animal with a polypeptide of this invention. Thus, an antibody directed against a polypeptide of this invention is contemplated.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED® antibody wherein the antigen-binding region of the antibody is derived from an antibody produced by immunizing macaque monkeys with the antigen of interest. Methods of making humanized antibodies are known in the art.

“Human antibodies” can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boemer et al. are also available for the preparation of human monoclonal antibodies. Cole et al., Monoclonal Antibodies and Cancer Therapy, Alan R. Liss, p. 77 (1985); Boemer et al., J. Immunol., 147(1):86-95 (1991).

A “composition” of this invention comprises a polypeptide of this invention, optionally in combination with a physiologically acceptable carrier. The composition can further comprise an additional therapeutic agent to treat the indication intended. In some embodiments, the composition comprises a second therapeutic agent selected from a drug for treating an immune-related disease and a drug for treating a cancer. In some embodiments, the drug for treating a cancer is selected from the group consisting of a cytotoxic agent, a chemotherapeutic agent, a growth inhibiting agent and a chemotherapeutic agent

“Carriers” as used herein include physiologically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONIC®.

The word “label” when used herein refers to a detectable compound or composition which is conjugated directly or indirectly to the polypeptide, antibody, antagonist or composition so as to generate a “labeled” a polypeptide, antibody, antagonist or composition. The label can be detectable by itself (e.g. radioisotope labels or fluorescent labels) or, in the case of an enzymatic label, can catalyze chemical alteration of a substrate compound or composition which is detectable (e.g., by FRET).

Various tag polypeptides and their respective antibodies are well known in the art. Tagged polypeptides and antibodies of this invention are contemplated. Examples include poly-histidine (poly-His) or poly-histidine-glycine (poly-His-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. The FLAG-peptide [Hopp et al., BioTechnology, 6:1204-1210 (1988)] is recognized by an anti-FLAG M2 monoclonal antibody (Eastman Kodak Co., New Haven, Conn.). Purification of a protein containing the FLAG peptide can be performed by immunoaffinity chromatography using an affinity matrix comprising the anti-FLAG M2 monoclonal antibody covalently attached to agarose (Eastman Kodak Co., New Haven, Conn.). Other tag polypeptides include the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidanine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and physiologically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and physiologically acceptable salts, acids or derivatives of any of the above.

A “growth inhibitory agent” when used herein refers to a compound or composition which inhibits growth of a cell, either in vitro or in vivo. Thus, the growth inhibitory agent is one that significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (W B Saunders: Philadelphia, 1995), especially p. 13.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie Blue or, preferably, silver stain. Isolated protein includes protein in situ within recombinant cells, since at least one component of the protein natural environment will not be present. Ordinarily, however, isolated protein will be prepared by at least one purification step.

A “heterologous” component refers to a component that differs from a reference component (e.g., if the reference component is referred to as naturally-occurring human BR3 sequence, a heterologous component will be different from a naturally occurring BR3 sequence). In one example, if a polynucleotide obtained from one organism differs from a polynucleotide sequence of a second organism and it is introduced by genetic engineering techniques into the polynucleotide sequence of a second organism (the reference component), then the polynucleotide derived from the first organism is heterologous to the polynucleotide of the second organism and which, if expressed, can encode a polypeptide which is heterologous to the respective polypeptide of the second organism Similarly, in some embodiments, a polypeptide that is fused to a second polypeptide that has a different function or sequence than the first peptide, is a heterologous to the second peptide. Heterologous components may also refer to chemically synthesized components, for example synthetic polypeptides.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

The term “therapeutically effective amount” refers to an amount of a composition of this invention effective to “alleviate” or “treat” a disease or disorder in a subject or mammal. Generally, alleviation or treatment of a disease or disorder involves the lessening of one or more symptoms or medical problems associated with the disease or disorder. In some embodiments, it is an amount that results in the reduction in the number of B cells in the mammal. In the case of cancer, the therapeutically effective amount of the drug can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. In some embodiments, a composition of this invention can be used to prevent the onset or reoccurrence of the disease or disorder in a subject or mammal. For example, in a subject with autoimmune disease, a composition of this invention can be used to prevent or alleviate flare-ups.

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Optionally, the cancer will express, or have associated with the cancer cell, BLyS. In some embodiments, the cancers for treatment herein include lymphoma, leukemia and myeloma, and subtypes thereof, such as Burkitt's lymphoma, multiple myeloma, acute lymphoblastic or lymphocytic leukemia, non-Hodgkin's and Hodgkin's lymphoma, and acute myeloid leukemia.

The term “immune related disease” means a disease in which a component of the immune system of a mammal causes, mediates or otherwise contributes to morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include 1, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

“Autoimmune disease” is used herein in a broad, general sense to refer to disorders or conditions in mammals in which destruction of normal or healthy tissue arises from humoral or cellular immune responses of the individual mammal to his or her own tissue constituents. Examples include, but are not limited to, lupus erythematous, thyroiditis, rheumatoid arthritis, psoriasis, multiple sclerosis, autoimmune diabetes, and inflammatory bowel disease (IBD).

As used herein, “B cell depletion” refers to a reduction in B cell levels in an animal or human after drug or antibody treatment, as compared to the level before treatment. B cell levels are measurable using well known assays such as by getting a complete blood count, by FACS analysis staining for known B cell markers, and by methods such as described in the Experimental Examples. B cell depletion can be partial or complete. In a patient receiving a B cell depleting drug, B cells are generally depleted for the duration of time when the drug is circulating in the patient's body and the time for recovery of B cells.

1. Polypeptide-BLyS Antagonists

The present invention describes polypeptides useful as antagonists of BLyS. In some embodiments, the 17-mer peptides are soluble (preferably not membrane bound), and may be used as core sequences or otherwise combined or conjugated with a variety of structures as is described below. Some amino acids in the 17-mer polypeptide were randomized and screened for functional conservative and non-conservative substitutions. As is understood by one of skill in the art and described herein, additions and substitutions may be accomplished without impairing the BLyS binding of the resulting 17mer peptide and constructs including the resulting 17mer peptide. Guidance as to allowed substitutions that yield BLyS binding function is provided below and in the examples. In some embodiments, residues implicated in structural or binding affinity relationships are conserved, meaning that either the amino acid identity is retained or a conservative substitution is made as described in the formulas and description below.

A polypeptide of this invention comprises a sequence selected from the group consisting of: Formula I (SEQ ID NO:1), Formula II (SEQ ID NO:18), Formula III (SEQ ID NO:26), a sequence recited in FIG. 12A-C, ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16) and ECFDLLVRHWVACGLLR (SEQ ID NO:17) and mixtures thereof.

In one aspect of the invention, a polypeptide comprises an amino acid sequence of Formula I:
X₁-C_{N}-X₃-D-X₅-L-X₇-X₈-X₉-X₁₀-X₁₁-X₁₂-C_{T}-X₁₄-X₁₅-X₁₆-X₁₇ (Formula I) (SEQ ID NO:1)

wherein X₁, X₃, X₅, X₇, X₈, X₉, X₁₀, X₁₁, X₁₂, X₁₄, X₁₅ and X₁₇ are any amino acid except cysteine; and

wherein X₁₆ is an amino acid selected from the group consisting of L, F, I and V; and

wherein the polypeptide does not comprise a cysteine within seven amino acid residues N-terminal to C_{N} (cysteine N terminal) and C-terminal to C_{T} (cysteine C terminal) of Formula I.

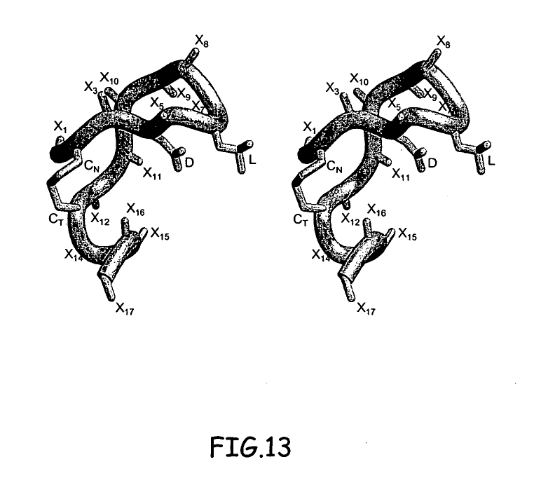

In some embodiments, a polypeptide comprising the sequence of Formula I has C_{N} and C_{T} joined by disulfide bonding; X₅LX₇X₈ forming the conformation of a type I beta turn structure with the center of the turn between L and X₇; and has a positive value for the dihedral angle phi of X₈. See FIG. 13 and description below. In some embodiments, X₁₀ is selected from the group consisting of W, F, V, L, I, Y, M and a non-polar amino acid. (SEQ ID NO:2). In some embodiments, X₁₀ is W. (SEQ ID NO:3). In some embodiments, X₃ is an amino acid selected from the group consisting of M, V, L, I, Y, F, W and a non-polar amino acid. (SEQ ID NO:4). In some embodiments, X₅ is selected from the group consisting of V, L, P, S, I, A and R. (SEQ ID NO:5). In some embodiments, X₇ is selected from the group consisting of V, T, I and L. (SEQ ID NO:6). In some embodiments, X₇ is not T or I. (SEQ ID NO:7). In some embodiments, X₈ is selected from the group consisting of any R, K, G, N, H and all D-amino acids. (SEQ ID NO:8). In some embodiments, X₉ is selected from the group consisting of H, K, A, R and Q. (SEQ ID NO:9). In some embodiments, X₁₁ is I or V. (SEQ ID NO:10). In some embodiments, X₁₂ is selected from the group consisting of P, A, D, E and S. (SEQ ID NO:11). In some embodiments, X₁₆ is L. (SEQ ID NO:12).

In specific embodiments, the sequence of Formula I is a sequence selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17).

Another aspect of the invention includes a polypeptide comprising an amino acid sequence of Formula II:
X₁-C_{N}-X₃-D-X₅-L-V-X₈-X₉-W-X₁₁-X₁₂-C_{T}-X₁₄-X₁₅-L-X₁₇ (Formula II)
(SEQ ID NO:18)

wherein X₁, X₃, X₅, X₈, X₉, X₁₁, X₁₂, X₁₄, X₁₅ and X₁₇ are any amino acid, except cysteine; and wherein the polypeptide does not comprise a cysteine within seven amino acid residues N-terminal to C_{N} (cysteine N terminal) and C-terminal to C_{T} (cysteine C terminal) of Formula II.

In some embodiments, a polypeptide comprising the sequence of Formula I has C_{N} and C_{T} joined by disulfide bonding; X₅LVX₈ forming the conformation of a type I beta turn structure with the center of the turn between L and X₇; and has a positive value for the dihedral angle phi of X₈. See FIG. 13.

In some embodiments, X₁, X₃, X₅, X₈, X₉, X₁₁, X₁₂, X₁₄, X₁₅ and X₁₇ are selected from a group of amino acids consisting of L, P, H, R, I, T, N, S, V, A, D, and G. (SEQ ID. NO:19).

In some embodiments of Formula II, X₃ is an amino acid selected from the group consisting of Norleucine, M, A, V, L, I, Y, F, W and a non-polar amino acid. (SEQ ID NO:20). In some embodiments of Formula II, X₅ is selected from the group consisting of V, L, P, S, I, A and R. (SEQ ID NO:21). In some embodiments of Formula II, X₈ is selected from the group consisting of R, K, G, N, H and all D-amino acids. (SEQ ID NO:22). In some embodiments of Formula II, X₉ is selected from the group consisting of H, K, A, R and Q. (SEQ ID NO:23). In some embodiments of Formula II, X₁₁ is selected from the group consisting of I and V. (SEQ ID NO:24). In some embodiments of Formula II, X₁₂ is selected from the group consisting of P, A, D, E and S. (SEQ ID NO:25).

The present invention also relates to a polypeptide comprising a sequence selected from any one of the sequences described in FIG. 12A-C. (SEQ ID NOS: 13, 15, 16, and 63-137).

Another aspect of the invention includes a polypeptide comprising an amino acid sequence of Formula III:
E-C_{N}-F-D-X₅-L-V-X₈-X₉-W-V-X₁₂-C_{T}-X₁₄-X₁₅-X₁₆-X₁₇ (Formula III) (SEQ ID NO:26)

wherein X₅, X₈, X₉, X₁₂, X₁₄, X₁₅ and X₁₇ are any amino acid except cysteine;

wherein X₁₆ is an amino acid selected from the group consisting of L, F, I and V;

wherein the polypeptide does not comprise a cysteine within seven amino acid residues N-terminal to C_{N} (cysteine N terminal) and C-terminal to C_{T} (cysteine C terminal) of Formula III; and

wherein C_{N} and C_{T} are joined by disulfide bonding.

In some embodiments of Formula III, the polypeptide comprising the contiguous sequence of Formula III has a disulfide bond between C_{N} and C_{T} and forms a type I beta turn structure with the center of the turn between L and V at X₅LVX₈; and has a positive value for the dihedral angle phi of X₈. See FIG. 13.

In some embodiments of Formula III, X₅, X₈, X₉, X₁₂, X₁₄, X₁₅ and X₁₇ are selected from the group consisting of L, P, H, R, I, T, N, S, V, A, D, and G. (SEQ ID NO:27). In some embodiments of Formula III, X₅ is L and X₈ is R. (SEQ ID NO:28). In some embodiments of Formula III, X₉ is selected from the group consisting of H, K, A, S, R and Q. In some embodiments of Formula III, X₁₂ is selected from the group consisting of P, A, D, E and S. (SEQ ID NO:30). In some embodiments of Formula III, X₁₂ is P. (SEQ ID NO:31). In some embodiments of Formula III, X₁₆ is L. (SEQ ID NO:32).

In specific embodiments, the sequence of Formula III is selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16) and ECFDLLVRHWVACGLLR (SEQ ID NO:17).

The present invention also relates to a polypeptide comprising a contiguous polypeptide sequence selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17). The present invention also relates to a polypeptide comprising a sequence selected from any one of the sequences described in FIG. 12A-C. Polypeptides comprising any one of the sequences described in FIG. 12A-C preferably join the cysteines of the sequence by disulfide bonding. In some embodiments, the sequence between the fifth and eighth residues of the sequence forms a conformation of a type I beta turn structure with the center of the turn between L and X₇ and the eighth residue has a positive value for the dihedral angle phi.

The present invention also relates to a polypeptide comprising at least 88% sequence identity with a contiguous 17mer polypeptide sequence selected from the group consisting of: ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), and ECFDLLVRHWVACGLLR (SEQ ID NO:17). In other embodiments sequence identity is at least 64%, and each successive integer to 100% after aligning to provide maximum homology. Homology is reduced for sequence gaps and sequences shorter than the 17mers of the present invention after aligning to provide maximum homology. Neither N-nor C-terminal extensions nor insertions shall be construed as reducing homology.

According to some embodiments of this invention, the polypeptide is less than 50 amino acids in length, less than 25 amino acids in length, or is a 17-mer.

In some embodiments, the polypeptides of this invention comprise additional polypeptide sequences N-terminal, C-terminal or both N-terminal and C-terminal to the sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C. The additional polypeptide sequences are heterologous to a native sequence BR3 polypeptide, and include, for example, Fc portion of immunoglobulins.

Another aspect of the invention involves polypeptides that comprise at least one and more preferably, more than one of a polypeptide comprising a sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C. The polypeptides that are linked together can have the same sequence or have different sequences. In some embodiments, these polypeptides can be joined to one another, optionally, through the use of a linker. The linker serves as a spacer and can be made of a variety of chemical compounds. In some embodiments, the linker is a polypeptide that has about 1 to 50 amino acids, more preferably about 1 to 30 amino acids. Linker sequences are known to those of skill in the art. For example, linker sequences include GGGKGGGG and GGGNSSGG and the like. In specific embodiments, the polypeptides linked together have the same sequence and comprise a formula: PP1-L1-PP1-L2-PP1, wherein PP1 has the same amino acid sequence and comprises an amino acid sequence selected from the group consisting of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), and sequences listed in FIG. 12A-C, and L1 and L2 are linker sequences that are different in sequence.

Antagonists for BLyS binding to BR3, such as the polypeptides described herein, preferably bind to BLyS with an affinity the same as or greater than a native BR3 sequence, such as BR3 ECD of (SEQ ID NO:60) or mini-BR3 of (SEQ ID NO:59). In some embodiments, the polypeptides having a sequence of that of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C have a binding affinity for BLyS of about 100 nM or less, preferably 10 nM or less, or 1 nM or less. One method of measuring binding affinity is provided in the examples.

A method used in the present invention to find BLyS antagonists involves identifying, modifying and selectively randomizing a core sequence of 17 residues. Specific techniques used are described further below and in the examples.

Structural considerations for 17-mer BLyS antagonists of the present invention include: In some embodiments, the N terminal cysteine residue (C_{N}) at position X₂ and C-terminal cysteine (C_{T}) at position X₁₃ are conserved and preferably form a disulfide bridge. In some embodiments, C_{N} and C_{T} are separated by 10 contiguous amino acids. Preferably, the 17mer sequence does not contain any cysteine residues other than at positions X₂ and X₁₃. Additionally, if the 17mer is included in a larger structure, sequences flanking the 17mer will preferably not include any cysteine residues within 7 amino acids of C_{N} or C_{T}. X₁₀ is substituted with any non-polar amino acid except for cysteine; for example: W, F, V, L, I, Y or M. In some embodiments, X₁₀ is W.

In some embodiments, the motif D-X₅-L-X₇ is conserved due to demonstrated contribution to BLyS binding. In some embodiments, a beta-turn located between C_{N} and C_{T}, is formed between X₄ and X₉. In some embodiments, the center of the beta-turn is positioned between L-X₇. In some embodiments, the structure of the 17mer peptides of the present invention is generally two beta-strands linked by a type I beta-turn, forming a beta-hairpin connected by a disulfide bond between C_{N} and C_{T}. In some embodiments, X₇ may be selected from the group consisting of V, T, I and L. In some embodiments, X₇ is preferably V. In some embodiments, the motif from X₄ to X₇ is DLLV.

Additionally, in some embodiments, the residue at X₈ adopts a positive value for the dihedral angle phi of X₈ to accommodate the type I beta turn in the beta hairpin structure. A stereoview of a model of the three-dimensional structure of a peptide of this invention is illustrated in FIG. 13. The model is based on solution NMR data acquired on two representative peptides, BLys0027 (SEQ ID NO:17) and BLyS0048 (SEQ ID NO:14). The peptide adopts a beta-hairpin structure: residues X₁-Asp and X₉-X₁₂ form beta strands that are connected by a type I beta-turn centered at Leu-X₇, with X₈ adopting a positive phi value. Residues X₁₄-X₁₇ are disordered in solution and can adopt more than one conformation. The backbone is shown as a ribbon diagram, with sidechains shown only for C_{N}, C_{T}, Asp, and Leu from Formula I; other positions are shown with a stick representation of the Calpha-Cbeta bond vector indicating the direction that the sidechain would be located. The beta-hairpin conformation shown in FIG. 13 can be defined by a variety of parameters measured by NMR spectroscopy. One parameter easily measured is the three-bond backbone coupling constant ³J_{HN-Hα}. In some embodiments, a peptide of this invention will have ³J_{HN-Hα} values of >8 Hz for residues in positions X₁, C_{N}, D, and X₁₁, >9 Hz for the residue X₇, and <7 Hz for residue C_{T}, measured at 20° C. in aqueous solution, indicating the peptide adopts a stable structure, consistent with the structure shown in FIG. 13. A more preferred peptide will have ³J_{HN-Hα} values of >8.5 Hz for residues in positions D and X₁₁, >9 Hz for residues X₁ and C_{N}, >10 Hz for residue X₇, and <6 Hz for residue C_{T}, measured at 20° C. in aqueous solution, indicating the peptide adopts a highly stable structure in solution, consistent with that shown in FIG. 13. Methods for determining the coupling constants using NMR techniques are known to those of skill in the art and are described in the Examples.

All D-amino acids and glycine readily adopt positive values for the backbone dihedral angle phi. In contrast, L-amino acids favor a negative phi value in most circumstances, including unstructured peptides and in the majority of proteins that have been visualized with high-resolution crystal structures. However, certain three-dimensional structural environments stabilize this more rare conformation of a positive value for the backbone dihedral angle phi. Specifically, in type I beta-turns that are embedded within a beta-hairpin structure, the positive phi value in the position analogous to that of X₈ in the 17mer peptides is required to maintain a stable beta-hairpin conformation [Nakamura, G. R., Starovasnik, M. A., Reynolds, M. E., and Lowman, H. B. (2001) Biochemistry 40, 9828-9835]. In some embodiments, X₈ is selected from the group consisting of L-amino acids R, K, G, N, H and all D-amino acids.

In some embodiments, the length of the binding region of the BLyS antagonist is 17 amino acids. In some embodiments, the polypeptide BLyS antagonist is 17 amino acids. In some embodiments, four amino acids, X₁₄-X₁₇, follow C_{T} at the C-terminal end. In some embodiments, X₁₆ forms a hydrophobic contact with BLyS when the 17mer is bound, therefore this residue is conserved. In some embodiments X₁₆ is L.

In an additional embodiment, the 17mer BLyS antagonist is ECFDLLVRHCVACGLLR (SEQ ID NO.216) corresponding to a contiguous 17 amino acid region of native human BR3.

2. Polynucleotides, Vectors, Host Cells

According to some embodiments, the polypeptides of this invention are selected from the group consisting of: 17mer peptides described herein, polypeptides incorporating one or more 17mer peptides as core regions, and covalently modified forms of the 17mer peptides and polypeptides (e.g., immunoadhesins, labeled polypeptides, protected polypeptides, conjugated polypeptides, fusion proteins, etc.). Various techniques that are employed for making these forms of polypeptides are described below. Methods for labeling polypeptides and conjugating molecules to polypeptides are known in the art.

The peptides and polypeptides or portions thereof can be made synthetically using methods of peptide synthesis. Synthetic methods of preparation may be especially useful to incorporate non naturally occurring amino acids at positions including D amino acids.

Compositions of the invention can be prepared using recombinant techniques known in the art. The description below relates to methods of producing such polypeptides by culturing host cells transformed or transfected with a vector containing the encoding nucleic acid and recovering the polypeptide from the cell culture. (See, e.g., Sambrook et al., Molecular Cloning: A Laboratory Manual (New York: Cold Spring Harbor Laboratory Press, 1989); Dieffenbach et al., PCR Primer: A Laboratory Manual (Cold Spring Harbor Laboratory Press, 1995)).

The nucleic acid (e.g., cDNA or genomic DNA) encoding the desired polypeptide may be inserted into a replicable vector for further cloning (amplification of the DNA) or for expression. Various vectors are publicly available. The vector components generally include, but are not limited to, one or more of the following: a signal sequence, an origin of replication, one or more marker genes, an enhancer element, a promoter, and a transcription termination sequence, each of which is described below. Optional signal sequences, origins of replication, marker genes, enhancer elements and transcription terminator sequences that may be employed are known in the art and described in further detail in WO97/25428.

Expression and cloning vectors usually contain a promoter that is recognized by the host organism and is operably linked to the encoding nucleic acid sequence. Promoters are untranslated sequences located upstream (5′) to the start codon of a structural gene (generally within about 100 to 1,000 bp) that control the transcription and translation of a particular nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to the encoding DNA by removing the promoter from the source DNA by restriction enzyme digestion and inserting the isolated promoter sequence into the vector.

Promoters suitable for use with prokaryotic hosts include the PhoA promoter, the β-galactosidase and lactose promoter systems, a tryptophan (trp) promoter system, T7 promoter, and hybrid promoters such as the tac, tacII or the trc promoter. However, other promoters that are functional in bacteria (such as other known bacterial or phage promoters) are suitable as well. For example, the nucleotide sequences have been published are known in the art. Methods of operably linking the promoters to target light and heavy chains are known in the art (Siebenlist et al. (1980) Cell 20: 269).

Construction of suitable vectors containing one or more of the above-listed components employs standard ligation techniques. Isolated plasmids or DNA fragments are cleaved, tailored, and re-ligated in the form desired to generate the plasmids required. For analysis to confirm correct sequences in plasmids constructed, the ligation mixtures can be used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced using standard techniques known in the art. [See, e.g., Messing et al., Nucleic Acids Res., 9:309 (1981); Maxam et al., Methods in Enzymology, 65:499 (1980)].

Expression vectors that provide for the transient expression in mammalian cells of the encoding DNA may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector [Sambrook et al., supra]. Transient expression systems, comprising a suitable expression vector and a host cell, allow for the convenient positive identification of polypeptides encoded by cloned DNAs, as well as for the rapid screening of such polypeptides for desired biological or physiological properties.

Other methods, vectors, and host cells suitable for adaptation to the synthesis of the desired polypeptide in recombinant vertebrate cell culture are described in Gething et al., Nature, 293:620-625 (1981); Mantei et al., Nature, 281:40-46 (1979); EP 117,060; and EP 117,058.

Suitable host cells for cloning or expressing the DNA in the vectors herein include prokaryote, yeast, or higher eukaryote cells. Suitable prokaryotes for this purpose include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Preferably, the host cell should secrete minimal amounts of proteolytic enzymes.

In addition to prokaryotes, eukaryotic microbes such as filamentous fungi or yeast are suitable cloning or expression hosts for vectors. Suitable host cells for the expression of glycosylated polypeptide are derived from multicellular organisms. Examples of all such host cells are described further in WO97/25428.

Host cells are transfected and preferably transformed with the above-described expression or cloning vectors and cultured in nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable; either as an extrachromosomal element or by chromosomal integrant. Depending on the host cell used, transformation is done using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. In addition, plants may be transfected using ultrasound treatment as described in WO 91/00358 published 10 Jan. 1991.

For mammalian cells without such cell walls, the calcium phosphate precipitation method of Graham and van der Eb, Virology, 52:456-457 (1978) may be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Prokaryotic cells can be cultured in suitable culture media as described generally in Sambrook et al., supra. Examples of commercially available culture media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: A Practical Approach, M. Butler, ed. (IRL Press, 1991).

The expressed polypeptides may be recovered from the culture medium as a secreted polypeptide, although may also be recovered from host cell lysates when directly produced without a secretory signal. If the polypeptide is membrane-bound, it can be released from the membrane using a suitable detergent solution (e.g. Triton-X 100) or its extracellular region may be released by enzymatic cleavage.

When the polypeptide is produced in a recombinant cell other than one of human origin, it is free of proteins or polypeptides of human origin. However, it is usually necessary to recover or purify the polypeptide from recombinant cell proteins or polypeptides to obtain preparations that are substantially homogeneous. As a first step, the culture medium or lysate may be centrifuged to remove particulate cell debris. The following are procedures exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, SEPHADEX® G-75; and protein A SEPHAROSE® columns to remove contaminants such as IgG.

3. Phage Display

According to some embodiments, the polypeptides of this invention selected from the group consisting of: Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), and sequences listed in FIG. 12A-C, may utilized in phage display.

Using the techniques of phage display allows the generation of large libraries of protein variants which can be rapidly sorted for those sequences that bind to a target molecule with high affinity. Nucleic acids encoding variant polypeptides are fused to a nucleic acid sequence encoding a viral coat protein, such as the gene III protein or the gene VIII protein. Monovalent phage display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phage display system, the gene fusion is expressed at low levels and wild type gene III proteins are also expressed so that infectivity of the particles is retained. Methods of generating peptide libraries and screening those libraries have been disclosed in many patents (e.g. U.S. Pat. No. 5,723,286, U.S. Pat. No. 5,432,018, U.S. Pat. No. 5,580,717, U.S. Pat. No. 5,427,908 and U.S. Pat. No. 5,498,530).

In some embodiments, Formula I (SEQ ID NO:1), Formula II (SEQ ID NO:18), or Formula III (SEQ ID NO:26) are expressed as peptide libraries on phage. The phage expressing the library of polypeptides of Formula I, Formula II or Formula III are then subjected to selection based on BLyS binding. In some embodiments, the selection process involves allowing some phage bind to biotinylated BLyS which is subsequently bound to a NEUTRAVIDIN® plate. Phage bound to the plate through the BLyS-biotin-neutravidin binding are recovered and propagated. In some embodiments, the phage are subject to several rounds of selection. In some embodiments, the phage is incubated with BLyS-biotin, followed by the addition of unbiotinylated BLyS as a competitive binder. Additional guidance of use of phage display in the context of the present invention is provided in the Examples.

4. Fusion Proteins and Conjugated Polypeptides

Immunoadhesin molecules comprising the polypeptides of this invention are further contemplated for use in the methods herein. In some embodiments, the molecule comprises a fusion of a polypeptide of this invention with an immunoglobulin or a particular region of an immunoglobulin. For a bivalent form of the immunoadhesin, such a fusion usefully comprises the Fc region of an IgG molecule. In a further embodiment, the Fc region is from a human IgG1 molecule. In some embodiments, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130 issued Jun. 27, 1995 and Chamow et al., TIBTECH, 14:52-60 (1996).

The simplest and most straightforward immunoadhesin design often combines the binding domain(s) of the adhesin (e.g. polypeptide of this invention) with the Fc region of an immunoglobulin heavy chain. For example, a polypeptide comprising a sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C can be covalently linked to an Fc portion of an immunoglobulin by recombinant methods. In addition, one or more of these polypeptides can be linked to one another and linked to an Fc portion of an immunoglobulin.

Ordinarily, when preparing the immunoadhesins of the present invention, nucleic acid encoding the binding domain of the adhesin will be attached in frame 3′ to the nucleic acid encoding the N-terminus of an immunoglobulin constant domain sequence such that a fusion protein comprising the adhesin and constant domain is produced upon expression. However N-terminal fusions are also possible.

Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G1 (IgG1). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and CH2 and CH3 or (b) the CH1, hinge, CH2 and CH3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:

(a) ACL-ACL;

(b) ACH-(ACH, ACL-ACH, ACL-VHCH, or VLCL-ACH);

(c) ACL-ACH-(ACL-ACH, ACL-VHCH, VLCL-ACH, or VLCL-VHCH)

(d) ACL-VHCH-(ACH, or ACL-VHCH, or VLCL-ACH);

(e) VLCL-ACH-(ACL-VHCH, or VLCL-ACH); and

(f) (A-Y)n-(VLCL-VHCH)2,

wherein each A represents identical or different polypeptides comprising an amino acid sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C or combinations thereof;

VL is an immunoglobulin light chain variable domain;

VH is an immunoglobulin heavy chain variable domain;

CL is an immunoglobulin light chain constant domain;

CH is an immunoglobulin heavy chain constant domain;

n is an integer greater than 1;

Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the CH2 domain, or between the CH2 and CH3 domains. Similar constructs have been reported by Hoogenboom et al., Mol. Immunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the polypeptide of this invention.

The polypeptides of the present invention can also be modified in a way to form chimeric molecules by fusing the polypeptide to another, heterologous polypeptide or amino acid sequence. According to some embodiments, such heterologous polypeptide or amino acid sequence is one which acts to oligomerize the chimeric molecule. In some embodiments, such a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an “-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

The polypeptide of the present invention may also be conjugated to an agent selected from the group consisting of a growth inhibitory agent, a cytotoxic agent, a detection agent, an agent that improves the bioavailability of the polypeptide and an agent that improves the half-life of the polypeptide. In some embodiments, the cytotoxic agent is a toxin, an antibiotic and a radioactive isotope. In additional embodiments, the polypeptide of the present invention is conjugated to a chemotherapeutic agent.

To increase the half-life of the immunoadhesins, antibodies or other polypeptides of this invention, one can attach a salvage receptor binding epitope to the antibody (especially an antibody fragment), immunoadhesin or polypeptide of this invention as described in U.S. Pat. No. 5,739,277, for example (e.g., the nucleic acid encoding the salvage receptor binding epitope can be linked in frame to a nucleic acid encoding a polypeptide sequence of this invention so that the fusion protein expressed by the nucleic acid molecule comprises the epitope and a polypeptide sequence of this invention). As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO00/42072 (Presta, L.). In another embodiment, the serum half-life can also be increased, for example, by attaching serum albumin or a portion of serum albumin that binds to the FcRn receptor or a serum albumin binding peptide described in WO01/45746 to an immunoadhesin, antibody or polypeptide of this invention. See also, Dennis, M. S., et al., (2002) JBC 277(38):35035-35043 for serum albumin binding peptide sequences.

5. Construction of Peptide-Polymer Conjugates

In some embodiments the strategy for the conjugation of a polymer, (e.g., PEGylation) of synthetic peptides consists of combining, through forming a conjugate linkage in solution, a peptide and a PEG moiety, each bearing a special functionality that is mutually reactive toward the other. The peptide-polymer conjugate may be useful, interalia, for increasing the half life of the peptides, increasing the amount of peptide delivered, in formulations for inhalation, for increasing the effective size of the peptides, for increasing solubility, for stabilizing the peptide against proteolytic attack, and for reducing immunogenicity.

The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

a. Peptide Reactive Sites

In some embodiments, a peptide is covalently bonded (conjugated) via one or more of the amino acid residues of the peptide to a terminal reactive group on the polymer, depending mainly on the reaction conditions, the molecular weight of the polymer, etc. In some embodiments, multiple peptides are conjugated to a polymer having two or more terminal reactive groups. The polymer with the reactive group(s) is designated herein as activated polymer. The reactive group selectively reacts with free amino or other reactive groups on the peptide. Potential reactive sites include: N-terminal amino group, epsilon amino groups on lysine residues, as well as other amino, imino, carboxyl, sulfhydryl, hydroxyl, and other hydrophilic groups. It will be understood, however, that the type and amount of the reactive group chosen, as well as the type of polymer employed, to obtain optimum results, will depend on the particular peptide employed to avoid having the reactive group react with too many particularly active groups on the peptide. In some embodiments, a reactive residue, (e.g., lysine (K), a modified, non-natural amino acid, or other small molecule) may be substituted at a position suitable for conjugation.

In some embodiments, the peptide comprises the sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C have a terminal reactive group. In some embodiments, the peptide comprises at least one and more preferably, more than one of a polypeptide comprising a sequence of Formula I (SEQ ID NO:1), Formula II (SEQ ID NO:18), Formula III (SEQ ID NO:26), ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C. The polypeptides that are linked together can have the same sequence or have different sequences and a terminal reactive group. In some embodiments, these polypeptides can be joined to one another, optionally, through the use of a linker.

While conjugation may occur at any reactive amino acid on the polypeptide, in some embodiments, the reactive amino acid is lysine, which is linked to the reactive group of the activated polymer through its free epsilon-amino group, or glutamic or aspartic acid, which is linked to the polymer through an amide bond. In some embodiments, the reactive amino acids of the peptide are not cysteine residues at positions X₂ and X₁₂.

The degree of polymer conjugation with each peptide will vary depending upon the number of reactive sites on the peptide, the molecular weight, hydrophilicity and other characteristics of the polymer, and the particular peptide derivatization sites chosen. In some embodiments, the conjugate has a final molar ratio of 1 to 10 polymer molecules per peptide molecule, but greater numbers of polymer molecules attached to the peptides of the invention are also contemplated. In other embodiments, the conjugate has a final molar ratio of 1 to 10 peptide molecules per polymer molecule, but greater numbers of peptides attached to the polymer molecules are also contemplated. In some embodiments, each conjugate contains one polymer molecule. The desired amount of derivatization is easily achieved by using an experimental matrix in which the time, temperature and other reaction conditions are varied to change the degree of substitution, after which the level of polymer substitution of the conjugates is determined by size exclusion chromatography or other means known in the art.

b. Activated Polymers

In some embodiments, the polymer contains only a single group which is reactive. This helps to avoid cross-linking of protein molecules. However, it is within the scope herein to maximize reaction conditions to reduce cross-linking, or to purify the reaction products through gel filtration or ion exchange chromatography to recover substantially homogenous derivatives. In some embodiments, the polymer is covalently bonded directly to the peptide without the use of a multifunctional (ordinarily bifunctional) crosslinking agent.

In other embodiments, the polymer contains two or more reactive groups for the purpose of linking multiple peptides to the polymer backbone. For example, a homobifunctional PEG molecule has a reactive group on each end of a linear PEG, such that a peptide is covalently attached at each end. In some embodiments, branched PEG molecules are used to provide multiple reactive sites for peptide conjugation. Again, gel filtration or ion exchange chromatography can be used to recover the desired derivative in substantially homogeneous form. The conjugation of two or more peptides to a polymer molecule may boost apparent affinity, through an avidity effect, for example when binding conjugated peptides of the present invention with cell surface expressed BLyS.

The covalent modification reaction may take place by any appropriate method generally used for reacting biologically active materials with inert polymers, preferably at about pH 5-9, more preferably 7-9 if the reactive groups on the peptide are lysine groups. Generally, the process involves preparing an activated polymer (the polymer typically having at least one terminal hydroxyl group to be activated), preparing an active substrate from this polymer, and thereafter reacting the peptide with the active substrate to produce the peptide suitable for formulation. The above modification reaction can be performed by several methods, which may involve one or more steps. Examples of modifying agents that can be used to produce the activated polymer in a one-step reaction include cyanuric acid chloride (2,4,6-trichloro-5-triazine) and cyanuric acid fluoride.

In some embodiments, the modification reaction takes place in two steps wherein the polymer is reacted first with an acid anhydride such as succinic or glutaric anhydride to form a carboxylic acid, and the carboxylic acid is then reacted with a compound capable of reacting with the carboxylic acid to form an activated polymer with a reactive ester group that is capable of reacting with the peptide. Examples of such compounds include N-hydroxysuccinimide, 4-hydroxy-3-nitrobenzene sulfonic acid, and the like, and preferably N-hydroxysuccinimide or 4-hydroxy-3-nitrobenzene sulfonic acid is used. For example, monomethyl substituted PEG may be reacted at elevated temperatures, preferably about 100-110° C. for four hours, with glutaric anhydride. The monomethyl PEG-glutaric acid thus produced is then reacted with N-hydroxysuccinimide in the presence of a carbodiimide reagent such as dicyclohexyl or isopropyl carbodiimide to produce the activated polymer, methoxypolyethylene glycolyl-N-succinimidyl glutarate, which can then be reacted with the GH. This method is described in detail in Abuchowski et al., Cancer Biochem. Biophys., 7: 175-186 (1984). In another example, the monomethyl substituted PEG may be reacted with glutaric anhydride followed by reaction with 4-hydroxy-3-nitrobenzene sulfonic acid (HNSA) in the presence of dicyclohexyl carbodiimide to produce the activated polymer. HNSA is described by Bhatnagar et al., Peptides: Synthesis-Structure-Func-tion. Proceedings of the Seventh American Peptide Symposium, Rich et al. (eds.) (Pierce Chemical Co., Rockford Ill., 1981), p. 97-100, and in Nitecki et al., High-Technology Route to Virus Vaccines (American Society for Microbiology: 1986) entitled “Novel Agent for Coupling Synthetic Peptides to Carriers and Its Applications.”

In some embodiments, covalent binding to amino groups is accomplished by known chemistries based upon cyanuric chloride, carbonyl diimidazole, aldehyde reactive groups (PEG alkoxide plus diethyl acetal of bromoacetaldehyde; PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG.). Carboxyl groups are derivatized by coupling PEG-amine using carbodiimide. Sulfhydryl groups are derivatized by coupling to maleimido-substituted PEG (e.g. alkoxy-PEG amine plus sulfosuccinimidyl 4-(N-maleimidomethyl)cyclohexane-1-carboxylate) as described in WO 97/10847 published Mar. 27, 1997, or PEG-maleimide commercially available from Nektar Technologies, San Carlos, Calif. (formerly Shearwater Polymers, Inc.). Alternatively, free amino groups on the peptide (e.g. epsilon amino groups on lysine residues) may be coupled to N-hydroxysucciminidyl substituted PEG (PEG-NHS available from Nektar Technologies;) or can be thiolated with 2-imino-thiolane (Traut's reagent) and then coupled to maleimide-containing derivatives of PEG as described in Pedley et al., Br. J. Cancer, 70: 1126-1130 (1994).

Many inert polymers, including but not limited to PEG, are suitable for use in pharmaceuticals. See, e.g., Davis et al., Biomedical Polymers: Polymeric Materials and Pharmaceuticals for Biomedical Use, pp. 441-451 (1980). In some embodiments of the invention, a non-proteinaceous polymer is used. The nonproteinaceous polymer is typically a hydrophilic synthetic polymer, i.e., a polymer not otherwise found in nature. However, polymers which exist in nature and are produced by recombinant or in vitro methods are also useful, as are polymers which are isolated from native sources. Hydrophilic polyvinyl polymers fall within the scope of this invention, e.g. polyvinyl alcohol and polyvinylpyrrolidone. Particularly useful are polyalkylene ethers such as polyethylene glycol (PEG); polyoxyalkylenes such as polyoxyethylene, polyoxypropylene, and block copolymers of polyoxyethylene and polyoxypropylene (PLURONIC®); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

The polymer prior to conjugation need not be, but preferably is, water soluble, but the final conjugate is preferably water-soluble. Preferably, the conjugate exhibits a water solubility of at least about 0.01 mg/ml, and more preferably at least about 0.1 mg/ml, and still more preferably at least about 1 mg/ml. In addition, the polymer should not be highly immunogenic in the conjugate form, nor should it possess viscosity that is incompatible with intravenous infusion, injection, or inhalation if the conjugate is intended to be administered by such routes.

The molecular weight of the polymer can range up to about 100,000 D, and preferably is at least about 500 D, or at least about 1,000 D, or at least about 5,000 D. In some embodiments, the PEG or other polymer has a molecular weight in the range of 5000 (5k) to 20,000 (20k) D. The molecular weight chosen can depend upon the effective size of the conjugate to be achieved, the nature (e.g. structure, such as linear or branched) of the polymer, and the degree of derivatization, i.e. the number of polymer molecules per peptide, and the polymer attachment site or sites on the peptide. In some embodiments, branched PEG's may used to induce a large increase in effective size of the peptides. PEG or other polymer conjugates may be utilized to increase half-life, increase solubility, stabilize against proteolytic attack, and reduce immunogenicity. In some embodiments, a single PEG molecule with molecular weight in the range of 5k to 40k is conjugated to one or more peptides, which is suitable for, for example, administration by inhalation.

Functionalized PEG polymers to modify the peptides of the invention are available from Nektar Technologies of San Carlos, Calif. (formerly Shearwater Polymers, Inc.). Such commercially available PEG derivatives include, but are not limited to, amino-PEG, PEG amino acid esters. PEG-N-hydroxysuccinamide chemistry (NHS), PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-xycarbonylimidazole, PEG-nitrophenyl carbonate, PEG tresylate, PEG-glycidyl ether, PEG-aldehyde, PEG vinylsulfone, PEG-maleimide, PEG-orthopyridyldisulfide, heterofunctional PEGs, PEG vinyl derivatives, PEG silanes, and PEG phospholides. The reaction conditions for coupling these PEG derivatives will vary depending on the protein, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer.

c. Characterization of Conjugates.

The conjugates may be characterized by SDS-PAGE, gel filtration, NMR, tryptic mapping, liquid chromatography-mass spectrophotometry, and in vitro biological assays. For example, the extent of PEG conjugation may be shown by SDS-PAGE and gel filtration, and then analyzed by NMR, which has a specific resonance peak for the methylene hydrogens of PEG. The number of PEG groups on each molecule can be calculated from the NMR spectrum or mass spectrometry. Polyacrylamide gel electrophoresis in 10% SDS is appropriately run in 10 mM Tris-HCl pH 8.0, 100 mM NaCl as elution buffer. To demonstrate which residue is PEGylated, tryptic mapping can be performed. Thus, PEGylated peptides are digested with trypsin at the protein/enzyme ratio of 100 to 1 in mg basis at 37° C. for 4 hours in 100 mM sodium acetate, 10 mM Tris-HCl, 1 mM calcium chloride, pH 8.3, and acidified to pH<4 to stop digestion before separating on HPLC NUCLEOSIL® C-18 (4.6 mm×150 mm, 5.mu., 100A). The chromatogram is compared to that of non-PEGylated starting material. Each peak can then be analyzed by mass spectrometry to verify the size of the fragment in the peak. The fragment(s) that carried PEG groups are usually not retained on the HPLC column after injection and disappear from the chromatograph. Such disappearance from the chromatograph is an indication of PEGylation on that particular fragment that should contain at least one lysine residue. PEGylated peptides may then be assayed for ability to bind to the BLyS by conventional methods.

In some embodiments, conjugates are purified by ion-exchange chromatography, (e.g, ion exchange HPLC. The chemistry of many of the electrophilically activated PEGs results in a reduction of amino group charge of the PEGylated product. Thus, high resolution ion exchange chromatography can be used to separate the free and conjugated proteins, and to resolve species with different levels of PEGylation. In fact, the resolution of different species (e.g. containing one or two PEG residues) is also possible due to the difference in the ionic properties of the unreacted amino acids. In one embodiment, species with difference levels of PEGylation are resolved according to the methods described in WO 96/34015 (International Application No. PCT/US96/05550 published Oct. 31, 1996). Heterologous species of the conjugates are purified from one another in the same fashion.

In some embodiments, PEG-N-hydroxysuccinamide (NHS) reacts with a primary amine (e.g. lysines and the N-terminus). In some embodiments, PEG-NHS reacts with a C-terminal lysine (K) of the polypeptide. In some embodiments, the lysine residue is added to the C-terminus of the 17-mer polypeptide, while in other embodiments, X₁₇ is substituted with lysine. In some embodiments, the polymer reacts with the N-terminus. In a preferred embodiment, the conjugate is generated by utilizing the derivatization and purification methods described in the Examples below.

In one aspect, the invention provides any of the above-described conjugates formed by its component parts, i.e. one or more peptide(s) covalently attached to one or more polymer molecule(s), without any extraneous matter in the covalent molecular structure of the conjugate.

6. Antibodies

It is contemplated that the polypeptides, such as Formula I, Formula II, Formula III, ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C of this invention will be used to create antibodies.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al, Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the B cell surface marker. Other such antibodies may bind a first B cell marker and further bind a second B cell surface marker. Alternatively, an anti-B cell marker binding arm may be combined with an arm which binds to a triggering molecule on a leukocyte such as a T-cell receptor molecule (e.g. CD2 or CD3), or Fc receptors for IgG (FcR), such as FcRI (CD64), FcRII (CD32) and FcRIII (CD16) so as to focus cellular defense mechanisms to the B cell. Bispecific antibodies may also be used to localize cytotoxic agents to the B cell. These antibodies possess a B cell marker-binding arm and an arm which binds the cytotoxic agent (e.g. saporin, anti-interferon-”, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Antibodies with more than two valencies are contemplated. For example, trispecific antibodies can be prepared. Tutt et al. J. Immunol. 147: 60 (1991).

Antibodies can be screened for binding affinity to the polypeptides described herein, BLyS or a polypeptide comprising a sequence of Formula I, Formula II, Formula III, ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16), ECFDLLVRHWVACGLLR (SEQ ID NO:17), or sequences listed in FIG. 12A-C using methods known to those of skill in the art. Antibodies generated herein can be screened for BLyS antagonist activity in various assays for assessing functional activity of BLyS as described herein. Competitive binding assays may be utilized to assay the relative binding affinity of the antibody as compared to other BLyS antagonists using methods known in the art.

7. Variation in Polypeptides and Variation in CD20 Antagonists and Antibodies

Variation in the 17-mers of the present invention is as described above in section 1. However additional variation in protein regions conjugated, fused or otherwise flanking the 17-mers, as well as agents used in combination with the BLyS antagonists of the present invention is possible as described herein. Additionally, amino acid sequence modification(s) of CD20 antagonists and antibodies described herein are contemplated. For example, it may be desirable to improve the binding affinity and/or other biological properties of the CD20 binding antibody or antagonist.

Amino acid sequence variants are prepared by introducing appropriate nucleotide changes into the nucleic acid, or by peptide synthesis. Such modifications include, for example, deletions from, and/or insertions into and/or substitutions of, residues within the amino acid sequences of the CD20 antibody or antagonist. Any combination of deletion, insertion, and substitution is made to arrive at the final construct, provided that the final construct possesses the desired characteristics. The amino acid changes also may alter post-translational processes of the CD20 antagonist, such as changing the number or position of glycosylation sites. A useful technique for identifying locations for mutagenesis is “alanine scanning mutagenesis”, described above.

Amino acid sequence insertions include amino- and/or carboxyl-terminal fusions ranging in length from one residue to polypeptides containing a hundred or more residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide or other conjugated molecule which increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists, such anti-CD20 antibody, include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described above in reference to amino acid classes, may be introduced and the products screened.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

8. Assay Methods and Methods for Inhibiting BLyS

Generally, the methods of the invention for inhibiting BLyS signaling in mammalian cells comprise exposing the cells to a desired amount of antagonist which fully or partially blocks BR3 interaction with BLyS. In some embodiments, the amount of antagonist employed will be an amount effective to affect the binding and/or activity of BLyS or BR3 to achieve a therapeutic effect. This can be accomplished in vitro or in vivo in accordance, for instance, with the methods described below and in the Examples. Exemplary conditions or disorders to be treated with such BLyS antagonists include conditions in mammals clinically referred to as autoimmune diseases, including but not limited to rheumatoid arthritis, multiple sclerosis, psoriasis, and lupus or other pathological conditions in which B cell response(s) in mammals is abnormally upregulated such as cancer. As shown in the Examples below, BLyS antagonists inhibited BR3 binding to BLyS. These results indicate that the polypeptides of this invention can inhibit BLyS signaling, including its effects on B cell survival and maturation, that blocking or inhibiting BLyS using the BLyS antagonists of this invention can have therapeutic utility for autoimmune diseases such as RA. Exemplary conditions or disorders to be treated with BCMA antagonists include immune-related diseases and cancer.

Diagnostic methods are also provided herein. For instance, the polypeptides of the invention can be used to detect BLyS in mammals or in vitro assays, including detection in mammals known to be or suspected of having a BLyS-related pathological condition or expressing abnormal amounts of BLyS (e.g., lupus patients and NZF/WF1 mice). According to some embodiments, polypeptides of this invention are used, e.g., in immunoassays to detect or quantitate BLyS in a sample. According to some embodiments, a sample, such as cells obtained from a mammal, can be incubated in the presence of a labeled polypeptide of this invention, and detection of the labeled polypeptide is performed. Such assays, including various clinical assay procedures, are known in the art, for instance as described in Voller et al., Immunoassays, University Park, 1981.

According to some embodiments, BLyS/BR3 binding studies can be carried out in any known assay method, such as competitive binding assays, direct and indirect sandwich assays, and immunoprecipitation assays. According to some embodiments, BLyS/BR3 binding assays are carried out as described herein, using the polypeptides of the invention in place of native sequence BR3. Cell-based assays and animal models can be used to further understand the interaction between the BLyS and BR3 and the development and pathogenesis of the conditions and diseases referred to herein.

In one approach, mammalian cells can be transfected with the BLyS and/or a polypeptide of this invention described herein, and the ability of the BLyS antagonists to stimulate or inhibit binding or activity of BLyS is analyzed. Suitable cells can be transfected with a polypeptide of this invention, and monitored for activity. Such transfected cell lines can then be used to test the ability of BLyS antagonists (e.g., drug candidates) to inhibit, for example, B-cell signalling (e.g., B cell proliferation, Ig secretion, etc.).

In addition, primary cultures derived from transgenic animals can be used in the cell-based assays. Techniques to derive continuous cell lines from transgenic animals are well known in the art. [see, e.g., Small et al., Mol. Cell. Biol., 5:642-648 (1985)].

One suitable cell based assay is the addition of epitope-tagged BLyS (e.g., AP or Flag) to cells that have or express a polypeptide of this invention, and analysis of binding (in presence or absence or prospective BLyS antagonists) by FACS staining with anti-tag antibody. In another assay, the ability of a BLyS antagonist to inhibit the BLyS induced proliferation of B cells is assayed. B cells or cell lines are cultured with BLyS in the presence or absence or prospective BLyS antagonists and the proliferation of B cells can be measured by, e.g., 3H-thymidine incorporation or FACS analysis.

The results of the cell based in vitro assays can be further verified using in vivo animal models. A variety of well known animal models can be used to further understand the role of the BLyS antagonists identified herein in the development and pathogenesis of for instance, immune related disease or cancer or B-cell depletion, and to test the efficacy of the candidate therapeutic agents. The in vivo nature of such models makes them particularly predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g. subcutaneous injection, tail vein injection, spleen implantation, intraperitoneal implantation, and implantation under the renal capsule.

Animal models for delayed type hypersensitivity provides an assay of cell mediated immune function as well. Delayed type hypersensitivity reactions are a T cell mediated in vivo immune response characterized by inflammation that does not reach a peak until after a period of time has elapsed after challenge with an antigen. These reactions also occur in tissue specific autoimmune diseases such as multiple sclerosis (MS) and experimental autoimmune encephalomyelitis (EAE, a model for MS). A suitable procedure is described in detail in Current Protocols in Immunology, unit 4.5.

An animal model for arthritis is collagen-induced arthritis. This model shares clinical, histological and immunological characteristics of human autoimmune rheumatoid arthritis and is an acceptable model for human autoimmune arthritis. Mouse and rat models are characterized by synovitis, erosion of cartilage and subchondral bone. The compounds of the invention can be tested for activity against autoimmune arthritis using the protocols described in Current Protocols in Immunology, above, units 15.5. See also the model using a monoclonal antibody to CD18 and VLA-4 integrins described in Issekutz, A. C. et al., Immunology, (1996) 88:569.

Additionally, the compositions of the invention can be tested on animal models for psoriasis like diseases. The compounds of the invention can be tested in the scid/scid mouse model described by Schon, M. P. et al., Nat. Med., (1997) 3:183, in which the mice demonstrate histopathologic skin lesions resembling psoriasis. Another suitable model is the human skin/scid mouse chimera prepared as described by Nickoloff, B. J. et al., Am. J. Path., (1995) 146:580.

Various animal models are well known for testing anti-cancer activity of a candidate therapeutic composition. These include human tumor xenografting into athymic nude mice or scid/scid mice, or genetic murine tumor models such as p53 knockout mice.

Recombinant (transgenic) animal models can be engineered by introducing the coding portion of the molecules identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g. baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA, 82, 6148-615 [1985]); gene targeting in embryonic stem cells (Thompson et al., Cell, 56, 313-321 [1989]); electroporation of embryos (Lo, Mol. Cel. Biol., 3, 1803-1814 [1983]); sperm-mediated gene transfer (Lavitrano et al., Cell, 57, 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89, 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals may be further examined for signs of immune disease pathology, for example by histological examination to determine infiltration of immune cells into specific tissues or for the presence of cancerous or malignant tissue.

Alternatively, “knock out” animals can be constructed which have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., L1 et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

9. Compositions and Formulations

The polypeptides and compositions described herein are preferably employed in a carrier. Suitable carriers and their formulations are described in Remington's Pharmaceutical Sciences, 16th ed., 1980, Mack Publishing Co., edited by Oslo et al. Typically, an appropriate amount of a physiologically-acceptable salt is used in the carrier to render the formulation isotonic. Examples of the carrier include saline, Ringer's solution and dextrose solution. The pH of the solution is preferably from about 5 to about 8, and more preferably from about 7.4 to about 7.8. It will be apparent to those persons skilled in the art that certain carriers can be more preferable depending upon, for instance, the route of administration and concentration of agent being administered. The carrier can be in the form of a lyophilized formulation or aqueous solution.

Acceptable carriers, excipients, or stabilizers are preferably nontoxic to cells and/or recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN®, PLURONIC® or polyethylene glycol (PEG).

The formulation can also contain more than one active compound as necessary for the particular indication being treated, preferably those with complementary activities that do not adversely affect each other. The antagonist also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Oslo, A. Ed. (1980).

The formulations to be used for in vivo administration should be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and (ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

10. Modes of Therapy

The polypeptides described herein are useful in treating various pathological conditions, such as immune related diseases or cancer. These conditions can be treated by inhibiting a selected activity associated with BLyS in a mammal through administration of one or more polypeptides of the invention.

Diagnosis in mammals of the various pathological conditions described herein can be made by the skilled practitioner. Diagnostic techniques are available in the art which allow, e.g., for the diagnosis or detection of cancer or immune related disease in a mammal. For instance, cancers can be identified through techniques, including but not limited to, palpation, blood analysis, x-ray, NMR and the like. Immune related diseases can also be readily identified. In systemic lupus erythematosus, the central mediator of disease is the production of auto-reactive antibodies to self proteins/tissues and the subsequent generation of immune-mediated inflammation. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint can induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjogren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology, which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet $ cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of anti-body or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barr syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+ T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus-Host-Disease (GVHD) are T lymphocyte-dependent; inhibition of T lymphocyte function is ameliorative.

Other diseases in which intervention of the immune and/or inflammatory response have benefit are Infectious disease including but not limited to viral infection (including but not limited to AIDS, hepatitis A, B, C, D, E) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e. as from chemotherapy) immunodeficiency), and neoplasia.

The polypeptides of this invention can be administered in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes. Optionally, administration can be performed through mini-pump infusion using various commercially available devices. The polypeptides of this invention can also be employed using gene therapy techniques that have been described in the art.

Effective dosages and schedules for administering the polypeptides of this invention can be determined empirically, and making such determinations is within the skill in the art. Single or multiple dosages can be employed. It is presently believed that an effective dosage or amount of a polypeptide of this invention used alone can range from about 1 mg/kg to about 100 mg/kg of body weight or more per day. Interspecies scaling of dosages can be performed in a manner known in the art, e.g., as disclosed in Mordenti et al., Pharmaceut. Res., 8:1351 (1991).

When in vivo administration of a polypeptide of this invention thereof is employed, normal dosage amounts can vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, can necessitate delivery in a manner different from that to another organ or tissue. Those skilled in the art will understand that the dosage of polypeptide that must be administered will vary depending on, for example, the mammal which will receive the antagonist, the route of administration, and other drugs or therapies being administered to the mammal.

Depending on the type of cells and/or severity of the disease, about 1 mg/kg to 15 mg/kg (e.g. 0.1-20 mg/kg) of polypeptide is an initial candidate dosage for administration, whether, for example, by one or more separate administrations, or by continuous infusion. A typical daily dosage might range from about 1:g/kg to 100 mg/kg or more, depending on the factors mentioned above. For repeated administrations over several days or longer, depending on the condition, the treatment is sustained until a desired suppression of disease symptoms occurs. However, other dosage regimens can be useful.

Optionally, prior to administration of any polypeptide, the mammal or patient can be tested to determine levels or activity of BLyS. Such testing can be conducted by ELISA or FACS of serum samples or peripheral blood leukocytes.

It is contemplated that yet additional therapies can be employed in the methods. The one or more other therapies can include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors which are known in the art and defined further with particularity in Section I above. In addition, therapies based on therapeutic antibodies that target tumor antigens such as RITUXAN® or HERCEPTIN® as well as anti-angiogenic antibodies such as anti-VEGF.

Preparation and dosing schedules for chemotherapeutic agents can be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent can precede, or follow administration of, e.g. a polypeptide of this invention, or can be given simultaneously therewith. The antagonist can also be combined with an anti-oestrogen compound such as tamoxifen or an anti-progesterone such as onapristone (see, EP 616812) in dosages known for such molecules.

It can be desirable to also administer antibodies against other antigens, such as antibodies which bind to CD20, CD11a, CD18, CD40, ErbB2, EGFR, ErbB3, ErbB4, or vascular endothelial factor (VEGF). Alternatively, or in addition, two or more antibodies binding the same or two or more different antigens disclosed herein can be co-administered to the patient. Sometimes, it can be beneficial to also administer one or more cytokines to the patient. In some embodiments, the antagonists herein are co-administered with a growth inhibitory agent. For example, the growth inhibitory agent can be administered first, followed by a polypeptide of the present invention.

The polypeptide of this invention (and one or more other therapies) can be administered concurrently or sequentially. Following administration of antagonist, treated cells in vitro can be analyzed. Where there has been in vivo treatment, a treated mammal can be monitored in various ways well known to the skilled practitioner. For instance, markers of B cell activity such as Ig production (non-specific or antigen specific) can be assayed.

11. Methods of Screening

The invention also encompasses methods of identifying BLyS antagonists. Such molecules can comprise small molecules or polypeptides, including antibodies. Examples of small molecules include, but are not limited to, small peptides or peptide-like molecules, preferably soluble peptides, and synthetic non-peptidyl organic or inorganic compounds. The screening assays for drug candidates are designed to identify compounds or molecules that bind or complex with the polypeptides identified herein, or otherwise interfere with the interaction of these polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

Assays for, for instance, antagonists are common in that they call for contacting the drug candidate with a polypeptide identified herein under conditions and for a time sufficient to allow these two components to interact.

In binding assays, the interaction is binding and the complex formed can be isolated or detected in the reaction mixture. In a particular embodiment, the polypeptide identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which can be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

Compounds or molecules that interfere with the interaction of BLyS and BR3 and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo can be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

The polypeptides of this invention or other BLyS antagonists can also be evaluated to determine the strength of their BLyS antagonist activity using assays known in the art. For example, BLyS antagonists may be evaluated by BLyS-dependent B cell proliferation and survival assays with primary human or primary murine B cells. Suitable assay formats for B Cell proliferation and survival assays are described in Do et al., (2000) J. Exp. Med. 192, 953-964; Khare et al., (2000) PNAS, 97, 3370-3375; and Moore et al., (1999) Science 285, 260-263.

In another assay, a BR3-DR4 chimeric receptor (the extracellular domain of human DR4 replaced with that of BR3) is used in an apoptosis assay. HeLa cells can be used for stable expression of BR3-DR4. Addition of BLyS triggers apoptosis due to activation of the BR3-DR4 chimeric receptor. The cell based screening is based the fact that BLyS antagonists should prevent BLyS induced cell death of these transfected cells. HeLa cells expressing BR3-DR4 were seeded into 12-well plate 16 hours before the assay. Purified recombinant BLyS (10 ng/ml) is first preincubated with the agents to be tested (e.g., a polypeptide of this invention) for 30 min at room temperature. 8 to 16 hours after addition of BLyS, cell death is quantified by Trypan-Blue assay.

12. Articles of Manufacture

In some embodiments of the invention, an article of manufacture containing materials useful for the treatment of the disorders described above is provided. The article of manufacture comprises a container and a label. Suitable containers include, for example, bottles, vials, syringes, and test tubes. The containers can be formed from a variety of materials such as glass or plastic. The container holds a composition which is effective for treating the condition and can have a sterile access port (for example the container can be an intravenous solution bag or a vial having a stopper pierceable by a hypodermic injection needle). The active agents in the composition comprises a polypeptide of this invention alone or in combination with an additional therapeutic agent. Examples of an additional therapeutic agent includes, chemotherapeutic agents, cytotoxic agents, etc. The label on, or associated with, the container indicates that the composition is used for treating the condition of choice. The article of manufacture can further comprise a second container comprising a physiologically-acceptable buffer, such as phosphate-buffered saline, Ringer's solution and dextrose solution. It can further include other materials desirable from a commercial and user standpoint, including other buffers, diluents, filters, needles, syringes, and package inserts with instructions for use.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

Commercially available reagents referred to in the Examples were used according to manufacturer's instructions unless otherwise indicated. The source of those cells identified in the following Examples, and throughout the specification, by ATCC® accession numbers is the American Type Culture Collection, Manassas, Va. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology 1991.

U.S. patent application Ser. No. ______ entitled “Combination Therapy for B Cell Disorders” (first inventor, Andrew C. Chan), filed Jun. 5, 2004, is hereby incorporated by reference. All other publications (including patents and patent applications) cited herein are hereby incorporated in their entirety by reference.

### EXAMPLES

### Example 1

### Materials

BLyS₈₂₋₂₈₅ production. A DNA fragment encoding human BAFF (residues 82-285) was cloned into the pET15b (Novagen) expression vector, creating a fusion with an N-terminal His-tag followed by a thrombin cleavage site. E. coli BL21(DE3) (Novagen) cultures were grown to mid-log phase at 37° C. in LB medium with 50 mg/L carbenicillin and then cooled to 16 C prior to induction with 1.0 mM IPTG. Cells were harvested by centrifugation after 12 h of further growth and stored at −80 C. The cell pellet was resuspended in 50 mM Tris, pH 8.0, and 500 mM NaCl and sonicated on ice. After centrifugation, the supernatant was loaded onto a Ni-NTA agarose column (Qiagen). The column was washed with 50 mM Tris, pH 8.0, 500 mM NaCl, and 20 mM imidazole and then eluted with a step gradient in the same buffer with 250 mM imidazole. BAFF-containing fractions were pooled, thrombin was added, and the sample was dialyzed overnight against 20 mM Tris, pH 8.0, and 5 mM CaCl₂ at 4° C. The protein was further purified on a monoQ (Pharmacia) column and finally on an S-200 size exclusion column in 20 mM Tris, 150 mM NaCl, and 5 mM MgCl₂. The resulting BLyS protein was used as described below.

BR3 extracellular domain production. The extracellular domain of human BR3 (residues 1 to 61) (SEQ ID NO:60) was subcloned into the pET32a expression vector (Novagen), creating a fusion with an N-terminal thioredoxin (TRX)-His-tag followed by an enterokinase protease site. E. coli BL21(DE3) cells (Novagen) were grown at 30° C. and protein expression induced with IPTG. TRX-BR3 was purified over a Ni-NTA column (Qiagen), eluted with an imidazole gradient, and cleaved with enterokinase (Novagen). BR3 was then purified over an S-Sepharose column, refolded overnight in PBS, pH 7.8, in the presence of 3 mM oxidized and 1 mM reduced glutathione, dialyzed against PBS, repurified over a MonoS column, concentrated, and dialyzed into PBS.

Peptide synthesis. MiniBR3 was synthesized as a C-terminal amide on a PERSEPTIVE BIOSYSTEMS® PIONEER™ Peptide Synthesizer (Applied Biosystems Inc.) using standard Fmoc chemistry. The side chain thiols of cysteines 19 and 32 were protected as trifluoroacetic acid (TFA)-stable acetamidomethyl (Acm) derivatives. Peptides were cleaved from the resin by treatment with 5% triisopropyl silane in TFA for 1.54 hr at room temperature. After removal of TFA by rotary evaporation, peptides were precipitated by addition of ethyl ether, then purified by reversed-phase HPLC (acetonitrile/H₂O/0.1% TFA). Peptide identity was confirmed by electrospray mass spectrometry. After lyophilization, the oxidized peptide was purified by HPLC. HPLC fractions containing reduced miniBR3 were adjusted to a pH of ˜9 with NH₄OH; the disulfide between cysteines 24 and 35 was then formed by addition of a small excess of K₃Fe(CN)₆, and the oxidized peptide purified by HPLC. Acm groups were removed (with concomitant formation of the second disulfide) by treatment of the HPLC eluate with a small excess of I₂ over ˜4 h. The progress of the oxidation was monitored by analytical HPLC, and the final product was again purified by HPLC. MiniBR3 was amino-terminally biotinylated on the resin by reaction with a 10-fold molar excess of sulfo-NHS-biotin (Pierce Chemical, Co.). The biotinylated miniBR3 was then cleaved from the resin and purified as described above for the unbiotinylated miniBR3.

The following peptides ECFDLLVRHWVACGLLR (BLyS0027) (SEQ ID NO:17), ECFDLLVRHWVPCGLLR (BLyS0048) (SEQ ID NO:14) and ECFDLLVRAWVPCSVLK (BLyS0051) (SEQ ID NO:13) were synthesized generally as follows. Peptides were synthesized on a RAININ® Symphony peptide synthesizer system using Rink amide resin and a threefold excess of 9-fluorenylmethoxycarbonyl (Fmoc) protected amino acid activated with 2-(1H-Benzotriazone-1-yl)-1,1,3,3-tetramethyluronium hexafluorophosphate (HBTU) in the presence of a fivefold excess of diisopropylethylamine (DIPEA). Amino acids were coupled twice at each position before deprotecting with a 20% solution of piperidine in dimethylformamide (DMF) and moving to the next residue. Washes between coupling steps were performed using dimethylacetamide (DMA). Following coupling of the final amino acid onto the peptide and its deprotection with 20% piperidine in DMF, the peptides were acylated at their amino terminus using 3 equivalents of acetic anhydride and 5 equivalents of DIPEA in DMA. Alternatively, the amino terminus was modified through acylation with 5-carboxyfluorescein, with (+)-biotin, or through reaction with another fluorophore or reporter molecule. The peptide was then cleaved from the resin through treatment with a solution of 95% trifluoroacetic acid (TFA) containing 2.5% water and 2.5% triisopropylsilane for 90 minutes. The volatiles were removed under reduced pressure, diethyl ether was added and the solids filtered off. The resulting precipitate was washed again with diethyl ether and the combined organics discarded. The washed solids were then washed successively with acetic acid, a 1:1 mixture of acetic acid and acetonitrile, a 1:1:1 mixture of acetic acid, acetonitrile and water, an 1:1:8 mixture of acetic acid, acetonitrile and water and finally with water. The combined washes were lyophilized and the resulting crude peptides purified using C18 reverse phase high performance liquid chromatography using a 30 minute 10% to 70% gradient of acetonitrile in water with 0.1% trifluoroacetic acid in each solvent at a flow rate of 15 milliliters per minute. Fractions containing the desired peptide were oxidized through addition of a saturated solution of iodine in acetic acid until the solution remained colored. This solution was then lyophilized. Finally, the lyophilized crude oxidized peptide was purified a second time under identical conditions and the fractions containing the desired peptide lyophilized. Some of the peptides were synthesized under identical conditions except that the synthesis was performed on a PERSEPTIVE BIOSYSTEMS® PIONEER™ Peptide Synthesizer (Applied Biosystems, Inc.) automated synthesizer using a fourfold excess of amino acid, coupling only once per residue.

### Example 2

### Phage Display of 17mers

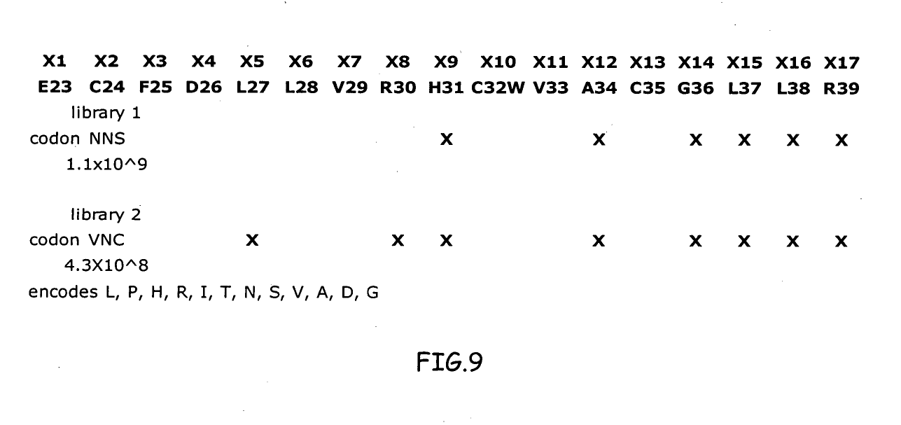

Library construction. A phagemid encoding the STII secretion signal sequence (“STII ss”), a linker (GGGSGGG, SEQ ID NO:61), and a sequence encoding the C-terminal residues of minor protein III of M13 phage (e.g., residues 267-421) (hereinafter, “cP3”) was used as a template for library construction. Two libraries were constructed using Kunkel mutagenesis techniques and oligonucleotides that introduced a fragment corresponding to residues 23-39 of human BR3 with a C32W mutation, also known as “17-mer C32W”, and additionally encoded mutations within the 17-mer C32W region. Specifically, library 1 encoded replacement codons at residues numbered 31, 34 and 36-39 (replacement codon: NNS=any codon), and library 2 encoded replacement codons residues 27, 30, 31, 34 and 36-39 (replacement codon: VNC=encodes amino acids L, P, H, R, I, T, N, S, V, A, D and G). In the replacement codons: N is 25% A, 25% C, 25% G, 25% T; S is 50% G/50% C; V is 33% G133% A/33% C; and C is 100% C. Library 1 encoded 1.1×10⁹ members and Library 2 encoded 4.3×10⁸ members. See FIG. 9.

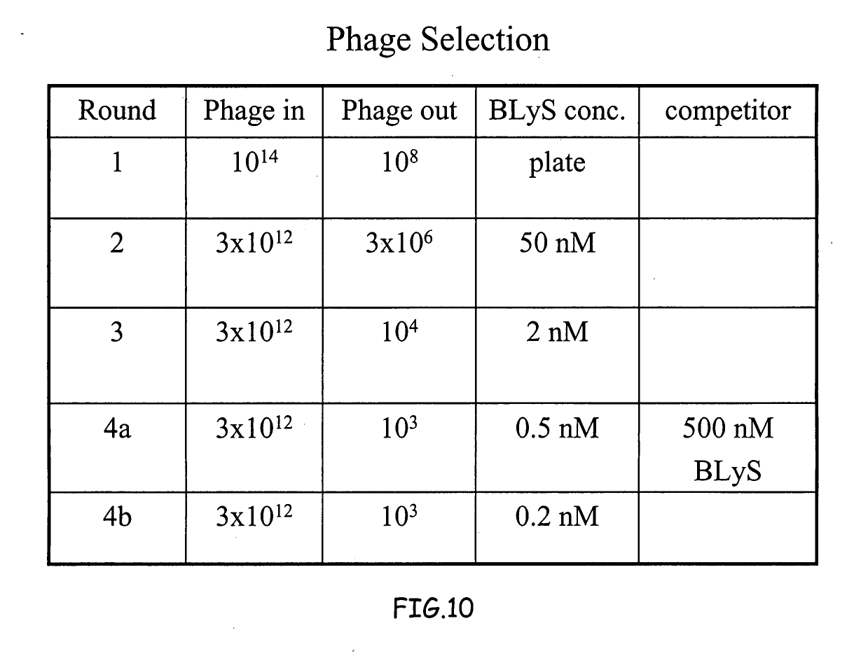

Library Sorting. The phage were subject to four rounds of selection (FIG. 10, overview). In general, the phage input per round was 10¹⁴ phage for the 1^{st} round (solid phase sorting) and 3×10¹² phage for additional rounds (solution phase sorting).

Phage Selection. The first round of selection was a solid phase sorting method. Maxisorp immunoplates (96-well) were coated with BLyS₈₂₋₂₈₅ prepared as described above (100 μl at 2 μg/ml in 50 mM carbonate buffer (pH 9.6)) overnight at 4° C. The wells were then blocked for one hour with 0.2% (w/v) BSA in phosphate-buffered saline (PBS) and washed 3-5 times with PBS, 0.05% TWEEN® 20. Phage particles ((100 μl/well in ELISA buffer (PBS/0.5% BSA/0.05% TWEEN® 20)) were added to the wells. After two hours, the wells were washed several times with PBS, 0.05% TWEEN® 20. The phage bound to the wells were eluted with 0.1N HCl for 10 min at RT. The eluted phage were neutralized by adding 1/20 volume 2M Tris pH 11.0.

To titer the phage, log phase XL-1 (OD 600 nm 0.3) was infected with eluted phage at 37° C. for 30 minutes. Next, the infected cells were serially diluted in 10 fold increments in 2YT. 10 μl aliquots of the infected cells were plated per carbenicillin plate. ˜10⁸ phage from each library were obtained from the first round of selection.

To propagate the phage, eluted phage was used to infect log phase XL-1 (OD 600 nm˜0.3) at 37° C. for 30 minutes. Helper phage, K07, and carbenicillin were added to the infection at a final concentration of 1×10¹⁰ pfu/ml KO7 and 50 ug/ml carbenicillin at 37° C. for another 30 minutes. The culture was grown in 2YT media with carbenicillin 50 ug/ml and 25 ug/ml kanamycin to final volumes of 25 ml at 37° C. overnight.

The phage were purified by spinning down the cells at 10000 rpm for 10 minutes. The supernatant was collected. 20% PEG/2.5M NaCl was added at ⅕ of the supernatant volume, mixed and allowed to sit at room temperature for 5 minutes. The phage were spun down into a pellet at 10000 rpm for 10 minutes. The supernatant was discarded and the phage pellet spun again for 5 minutes at 5000 rpm. The pellets were resuspended in 0.7 ml PBS and spun down at 13000 rpm for 10 minutes to clear debris. The OD of the resupended phage pellet was read at 268 mm.

The second-fourth rounds of selection utilized solution sorting methods. For the second round, NUNC® Maxisorp 96-well plates were coated with 5 ug/ml NEUTRAVIDIN® (Pierce Biotechnology, Inc.) at 4° C. overnight. Next, the plate was blocked with 200 μl/ml SUPERBLOCK® (Pierce Biotechnology, Inc.) in PBS for 30 min at room temperature. TWEEN® 20 was added to each well for a final concentration of 0.2% (v/w) and blocked for another 30 minutes at room temperature. The amplified, purified phage from the first round of selection were incubated with 50 nM biotinylated BLyS (final concentration) in 150 ul buffer containing SUPERBLOCK® 0.5% and 0.1% TWEEN® 20 for 1 h at room temperature. The mixtures were then diluted 5-10× with PBS/0.05% TWEEN® and applied at 100 μl/well to the NEUTRAVIDIN® coated plate. The plate was gently shaken for five minutes at room temperature to allow phage bound to biotinylated BLyS to be captured in the wells. The wells were then washed with PBS/0.05% TWEEN® 20 several times. Bound phage were eluted with 0.1N HCl for 10 min, neutralized, tittered, propagated and purified as described above. ˜3×10⁶ phage from each library were obtained from the second round of selection.

The third round of selection was similar to the second round, except a concentration of 2 nM biotinylated BLyS was incubated with the phage prior to dilution and addition to each well. Bound phage were eluted with 0.1N HCl for 10 min, neutralized, titered and propagated as described above. ˜10⁴ phage from each library were obtained from the third round of selection.

Phage from the third round of selection were next subjected to two different selection methods in the fourth round. Method 4a was similar to the second and third rounds of selection except that the phage was incubated in the presence of 0.5 nM biotinylated BLyS for 1 h at room temperature. The mixture was then incubated for an additional 15 minutes at room temperature in the presence of 1000 fold excess (500 nM) of unbiotinylated BLyS prior to dilution and addition to the coated wells Method 4b was also similar to the second and third rounds of selection except that 0.2 nM BLyS was incubated with the phage before dilution and addition to each well. Bound phage from each round four selection were eluted with 0.1N HCl for 10 min, neutralized, titered and propagated as described above. ˜10³ phage were obtained for each library from each of the fourth rounds (4a and 4b) of selection.

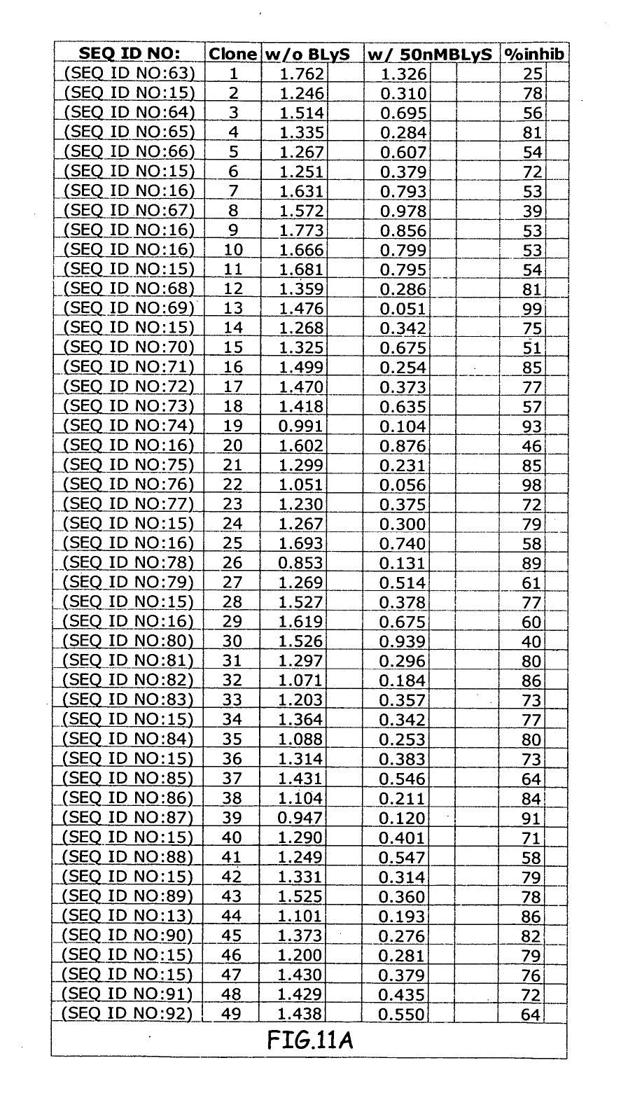

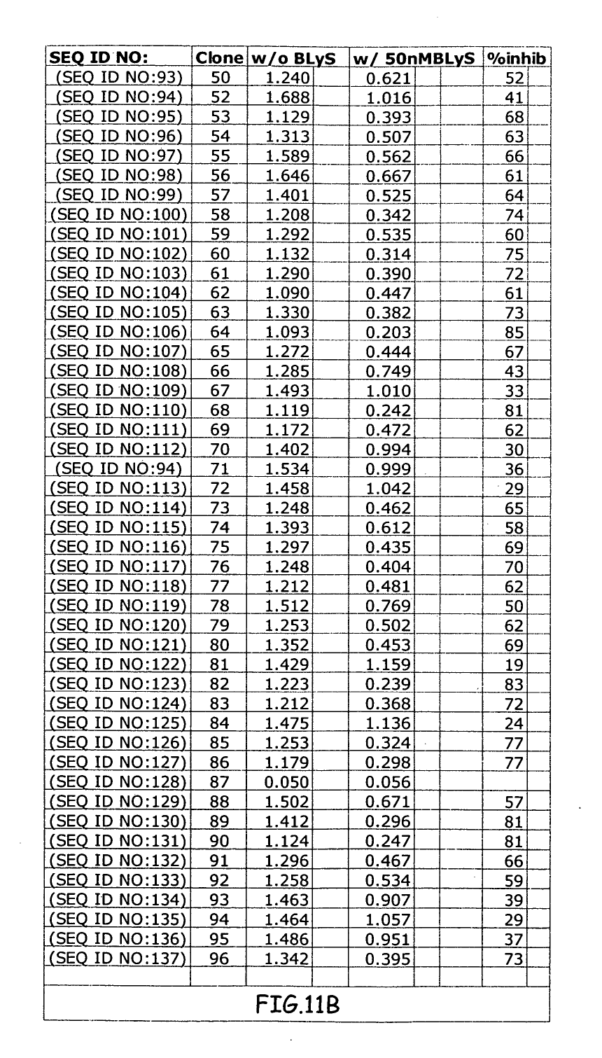

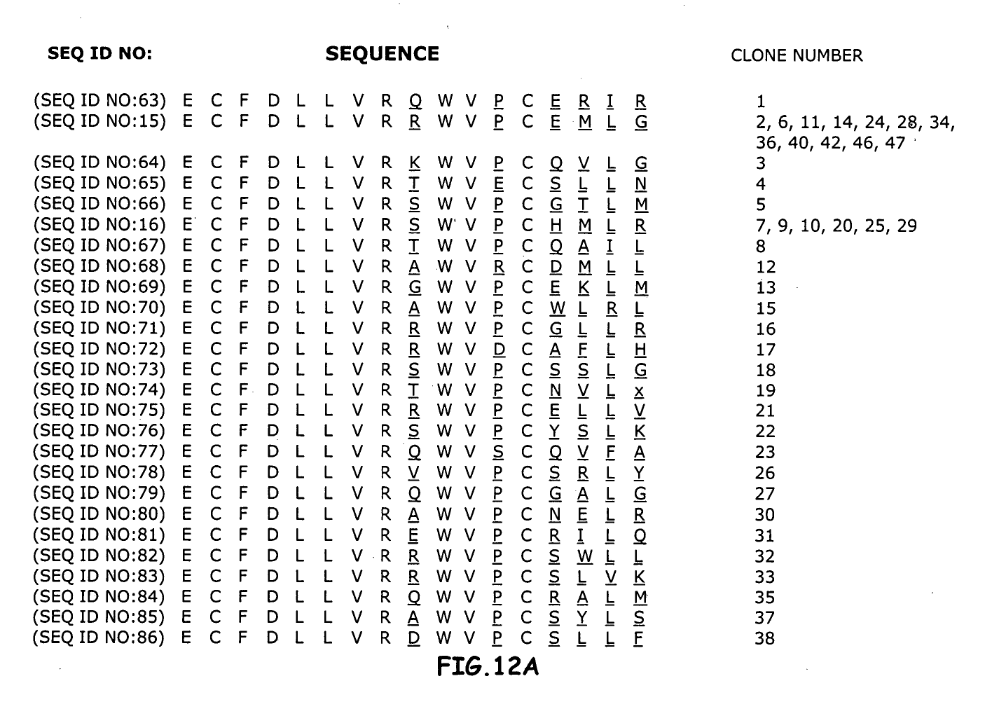

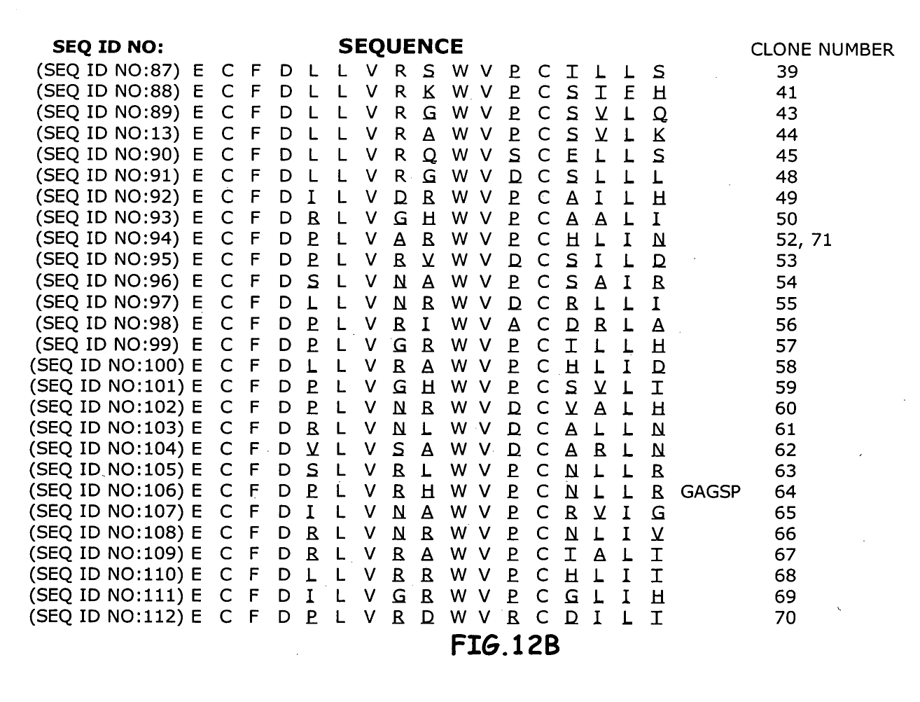

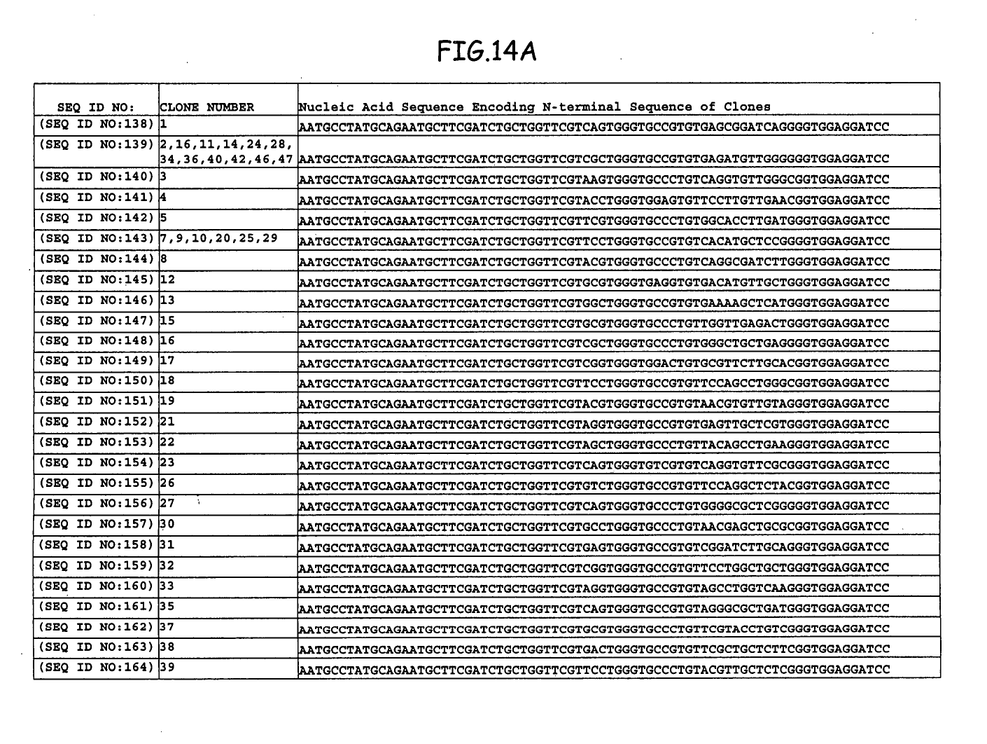

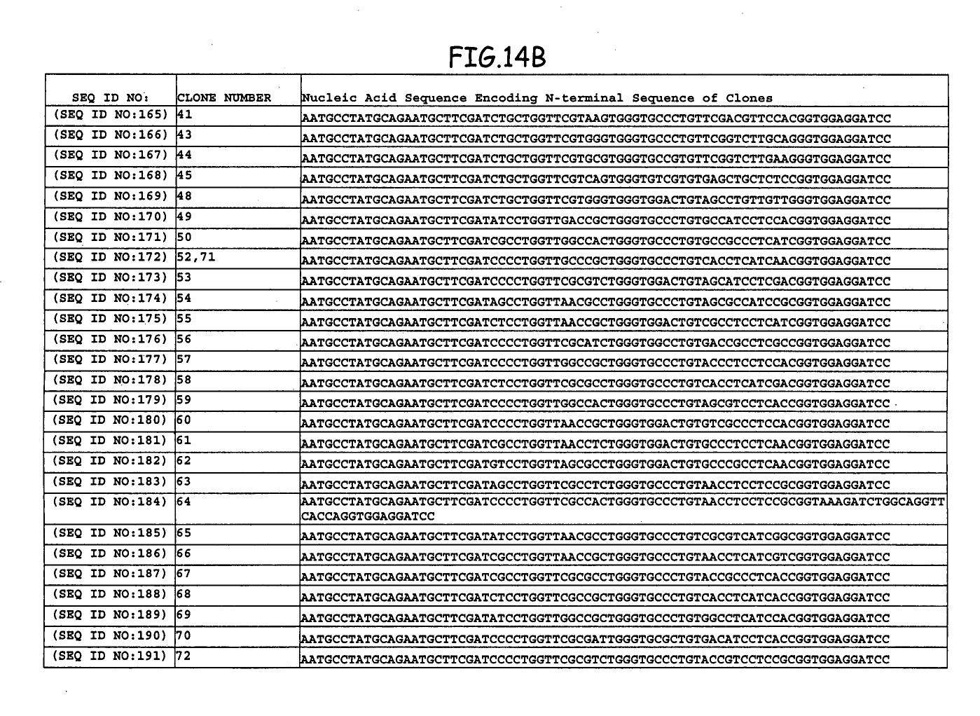

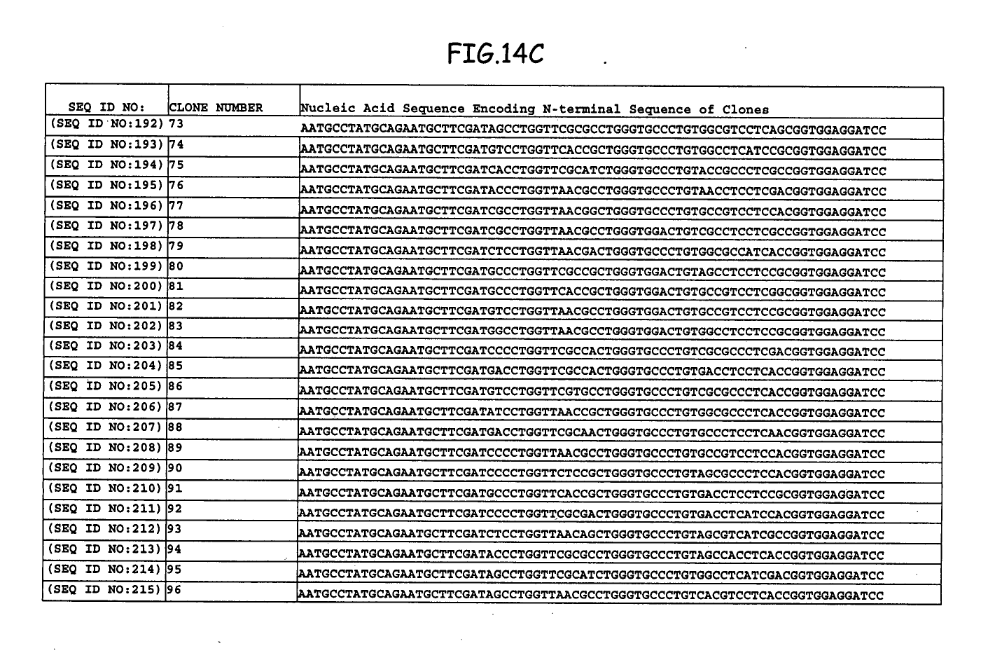

Clone Analysis. After the fourth round of selection, individual clones were grown in a 96-well format in 400 μL of 2YT medium supplemented with carbenicillin and KO7 helper phage. Supernatants from these cultures were used in phage ELISAs. For phage ELISAs, NUNC® Maxisorp 96-well plates were coated overnight at 4° C. with 100 μl of a 2 μg/ml solution of BLyS in carbonate buffer, pH 9.6. The plate was washed with PBS and blocked with 0.5% BSA in PBS for two hours. Phage supernatant was diluted 1:4 in ELISA binding buffer (PBS, 0.5% BSA, 0.05% TWEEN® 20) in the absence or presence of 50 nM BLyS and incubated for 1 h at RT. 100 ul of the diluted phage supernatants were then transferred to the coated plates and allowed to shake gently to capture phage for 20 minutes. The plates were then washed with PBS/0.05% TWEEN® 20 several times. 100 μl per well of HRP-conjugated anti-M13 antibody in PBS/0.05% TWEEN® 20 (1:5000) was then transferred to the plates and incubated for 20 min. After washing with PBS/0.05% TWEEN® followed by PBS, the plate was incubated 5 min with 100 μl PBS substrate solution containing 0.8 mg/ml OPD (Sigma) and 0.01% H₂O₂. The reaction was quenched with 100 μl/well 1M H₃PO₄ and the plate read at 490 nm. FIG. 11 reports the results of the phage ELISA in the absence and presence of 50 nM BLyS, as well as a calculation of % inhibition calculated for this BLyS concentration for each of the 96 phage clones. The clones tested were then sequenced as previously described (Weiss, G. A., Watanabe, C. K., Zhong, A., Goddard, A., and Sidhu, S. S. (2000) Proc. Natl. Acad. Sci. U.S.A. 97, 8950-8954). Sequences of acceptable quality were translated and aligned. The amino acid sequences of the 17mers are shown in FIG. 12A-C. The nucleic acid sequences encoding, among other things, the 17-mer sequences are provided in FIG. 14A-C. The nucleic acid sequences for each entry in FIG. 14A-C can be translated to the following amino acid sequences: part of the leader sequence from the StII secretion signal, NAYA, 17mer sequence described above (in FIG. 12A-C), and part of a linker sequence (GGGS).

Fourteen clones were further analyzed in a BLyS binding assay to determine their IC50 value. Clones 2 and 7 had a high number of siblings (clones with an identical sequence) in the fourth round. According to the phage ELISA assay, clones 13, 19, 22, 26, 32, 39 and 44 were greatly inhibited from binding to the plate by 50 nM BLyS (FIG. 11). The binding of clones 35, 45, 68, 82 and 90 was also greatly inhibited in the phage ELISA assay (FIG. 11). Phage supernatants from these 14 clones were used to infect log phase XL-1 which were propagated and purified as described above.

To normalize for display and phage yield and determine the appropriate dilution of phage for IC50 measurement, serial dilutions of purified phage from each clone were incubated in ELISA binding buffer (PBS, 0.5% BSA, 0.05% TWEEN® 20) for 1 hour at room temperature. 100 μl of each dilution were transferred to BLyS coated plates and allowed to shake gently to capture phage for 20 minutes as described above. Bound phage was detected by HRP-conjugated anti-M13 antibody, followed by OPD/H₂O₂ substrate reaction, quenched and read at 490 nm as described above. By this process, the dilution of each clone that yielded ˜1 O.D. at 490 nm was determined and used in the IC50 assay.

To determine the IC50 value of each of the 14 clones, NUNC® Maxisorp 96-well plates were coated overnight at 4° C. with 100 μl of a 2 μg/ml solution of BLyS in carbonate buffer, pH 9.6, and washed and blocked as described above. A dilution of amplified, purified phage for each of the 14 clones was incubated in the presence of a concentration series of BLyS ranging from 0.003-1000 nM in 130 ul ELISA binding buffer (PBS, 0.5% BSA, 0.05% TWEEN® 20) for 1 hour at room temperature. 100 μl of each of these concentration series were transferred to BLyS coated plates and captured, washed, detected with HRP-conjugated anti-M13 antibody and processed as described above. The results are shown in FIG. 15. IC50 values were determined by a four-parameter fit of the ELISA signal for each of the 14 clones. The IC50 values ranged from 0.4 (clone 44) to 11 nM (clone 22).

Competitive Displacement ELISA. The following 17-mers, Ac-ECFDLLVRHWVACGLLR-NH₂ (SEQ ID NO:17) (“BLyS0027”), Ac-ECFDLLVRHWVPCGLLR-NH₂ (SEQ ID NO:14) (“BLyS0048”), Ac-ECFDLLVRAWVPCSVLK-NH₂ (SEQ ID NO:13) (“BLyS0051”) were synthesized as described above. NUNC® Maxisorp 96-well plates were coated overnight at 4° C. with 100 μl of a 2 μg/ml solution of BLyS in carbonate buffer, pH 9.6. The plate was washed with PBS and blocked with 1% skim milk in PBS. Serial dilutions of the BR3 ECD (residues 1-61) (SEQ ID NO: 60) and the above 17-mer peptides were prepared in PBS/0.05% TWEEN® 20 containing 3 ng/ml biotinylated miniBR3. After washing with PBS/0.05% TWEEN®, 100 μl/well of each dilution was transferred and incubated for 1 hour at room temperature. The plate was washed with PBS/0.05% TWEEN® and incubated 15 min with 100 μl/well of 0.1 U/ml Streptavidin-POD (Boehringer Mannheim) in PBS/0.05% TWEEN®. After washing with PBS/0.05% TWEEN® followed by PBS, the plate was incubated 5 min with 100 μl PBS substrate solution containing 0.8 mg/ml OPD (Sigma) and 0.01% H₂O₂. The reaction was quenched with 100 μl/well 1M H₃PO₄ and the plate read at 490 nm. IC₅₀ values were determined by a four-parameter fit of the competitive displacement ELISA signal. The concentrations of initial stock solutions of miniBR3 and BR3 extracellular (SEQ ID NO: 60) domain were determined by quantitative amino acid analysis.

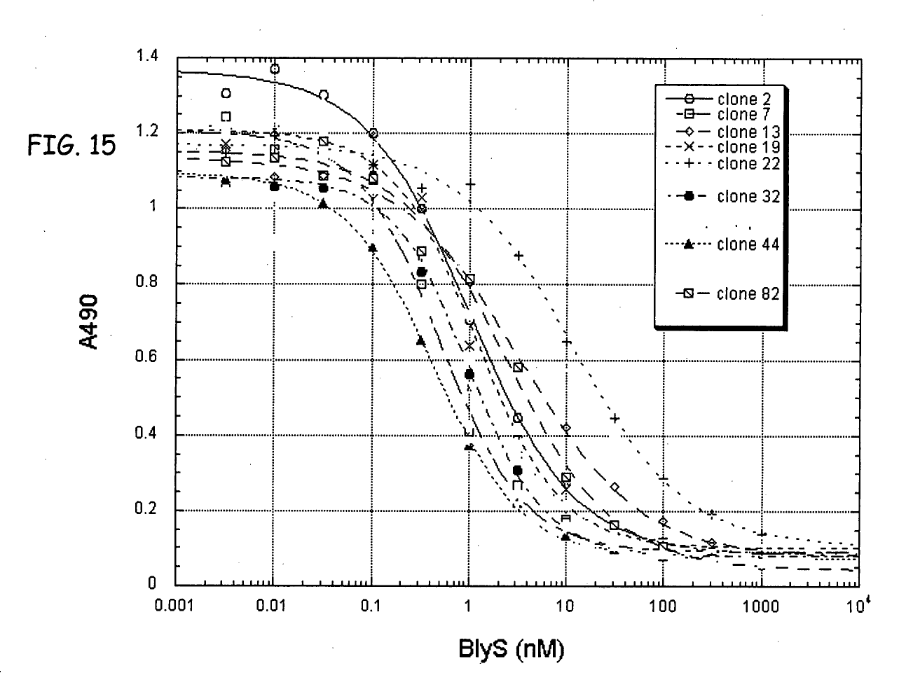

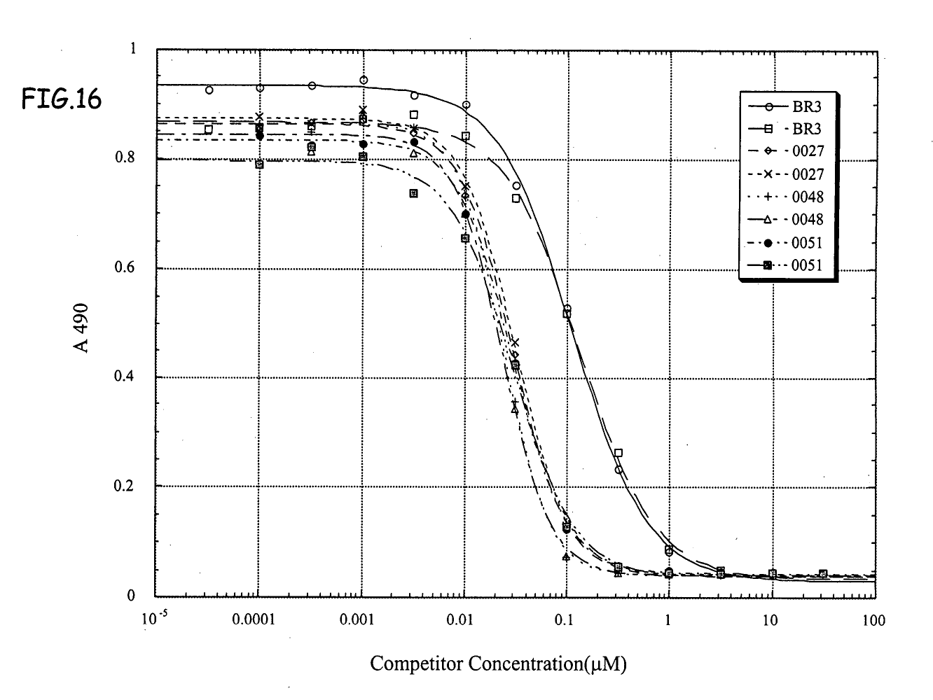

FIG. 16 shows that the IC50 values of BR3 ECD (SEQ ID NO: 60), BLyS0027 (SEQ ID NO:17), BLyS0048 (SEQ ID NO:14) and BLyS0051 (SEQ ID NO:13) using this assay. The 17-mer peptides all had greater affinity for BLyS than the 61-mer BR3 ECD (SEQ ID NO: 60).

### Example 3

The following peptides: Ac-ECFDLLVRHWVACGLLR-NH₂ (BLyS0027) (SEQ ID NO:17), ECFDLLVRHWVPCGLLR (BLyS0048) (SEQ ID NO:14) were analyzed by 2D NMR spectroscopy. Backbone HN-Halpha coupling constants are given in Table 2 below. Backbone ³J_{HN-Hα} coupling constants were measured from 2D COSY spectra acquired on a 500 MHz (blys048) or 600 MHz (blys027) spectrometer at 20° C. as described [Starovasnik, M. A., Skelton, N. J., O'Connell, M. P., Kelley, R. F., Reilly, D., and Fairbrother, W. J. (1996) Biochemistry 35, 15558-15569]. ov indicates that the relevant peak was overlapped, hence an accurate value of the coupling constant could not be obtained. na indicates the value could not be measured in this spectrum. NMR samples were prepared by dissolving lyophilized peptide in 92% H2O/8% D20 at a concentration of ˜3 mg/ml peptide. 2D NOESY, TOCSY, and COSY spectra were collected and used to assign all ¹H resonances using standard 2D NMR methods [Wüthrich, K. (1986) NMR of Proteins and Nucleic Acids (New York, J. Wiley and Sons)].

Three-bond backbone coupling constants are highly sensitive indicators of the three-dimensional structure and stability of a given polypeptide. The values shown for BLyS0027 (SEQ ID NO:17) and BLyS0048 (SEQ ID NO:14) indicate that each of these peptides adopts a highly stable structure in solution, and indicate the peptide adopts a turn conformation very similar to that seen in bhpBR3 (Kayagaki et al., 2002), the BR3 NMR structure (Gordon et al., Biochemistry 42, 5977-5983 2003) and BR3 from the BLyS/BR3 co-crystal structures (Liu et al., Nature 423, 49-56, 2003; Kim et al., 2003 Nature Structual Biology, 10, 342-348).

TABLE 2

| | | | ³J_{HN-Hα} | ³J_{HN-Hα} |
|---|---|---|---|---|
| | | | (Hz) | (Hz) most |
| | ³J_{HN-Hα} | ³J_{HN-Hα} | preferred | preferred for |
| | (Hz) | (Hz) | for peptide | peptide of |
| Residue | BLyS0027 | BLyS0048 | of invention | invention |
| X₁ (Glu) | 9.5 | 9.5 | >8 | >9 |
| C_{N} (Cys) | 9.9 | 10.0 | >8 | >9 |
| X₃ (Phe) | 7.2 | 7.2 | | |
| Asp | 8.9 | 9.7 | >8 | >8.5 |
| X₅ (Leu) | 6.0 | 5.9 | | |
| Leu | 5.9 | 6.4 | | |
| X₇ (Val) | 11.3 | 11.1 | >9 | >10 |
| X₈ (Arg) | 7.5 | 7.5 | | |
| X₉ (His) | 7.9 | 7.2 | | |
| X₁₀ (Trp) | 7.2 | 7.3 | | |
| X₁₁ (Val) | 9.2 | 9.3 | >8 | >8.5 |
| X₁₂ | 5.5 | na (Pro) | | |
| (Ala, blys027; | | | | |
| Pro, blys048) | | | | |
| C_{T} (Cys) | 5.7 | 5.4 | <7 | <6 |
| X₁₄ (Gly) | na | na | | |
| X₁₅ (Leu) | 6.9 | ov | | |
| X₁₆ (Leu) | 7.7 | 7.0 | | |
| X₁₇ (Arg) | 7.8 | ov | | |

### Example 4

This example demonstrates the synergy between anti-CD20 mAb and BR3 antagonist treatments for B cell modulation/depletion.

Materials and Methods:

FVB mice expressing a bacterial artificial chromosome encoding human CD20 (designated as hCD20⁺ mice) were treated with intraperitoneal injections of anti-CD20 mAb (single injection of 100 micrograms on Day 9), BR3-Fc (100 micrograms every other day from Days 1 through 12), or the combination of anti-CD20 mAb and BR3-Fc. Each group consisted of 4 mice. Two days following the last injection, the mice were sacrificed and analyzed for hCD20⁺ B cells. FACS analysis of spleen, blood, lymph node and Peyer's Patches were analyzed for B cell markers (CD21⁺CD23⁺).

Results:

1. Anti-CD20 mAb therapy depletes >99% of mature circulating B cells in the blood and lymph nodes.
2. BR3-Fc decreases mature circulating B cells in the blood and lymph nodes.
3. Anti-CD20 mAb therapy depletes T2 and follicular B cells, but not marginal zone B cells in the spleen.
4. BR3-Fc decreases T2/follicular and marginal zone B cells in the spleen.
5. The combination of anti-CD20 mAb and BR3-Fc synergizes to deplete all populations of B cells in the spleen.

These results and others are shown in U.S. patent application Ser. No. ______ entitled, “Combination Therapy for B Cell Disorders,” filed on the same day as the instant application (first inventor: Andrew C. Chan) and are specifically incorporated by reference herein.

A similar experiment performed in Cynomolgus monkeys is in progress. This experiment and other experiments demonstrate the surprising results that the combination of anti-CD20 mAb and BR3-Fc result in great synergy in depleting all subsets of B cells.

### Example 5

### Peptide-PEG Conjugates

BLyS₈₂₋₂₈₅ production. A DNA fragment encoding human BAFF (residues 82-285) was cloned into the pET15b (Novagen) expression vector, creating a fusion with an N-terminal His-tag followed by a thrombin cleavage site. E. coli BL21(DE3) (Novagen) cultures were grown to mid-log phase at 37° C. in LB medium with 50 mg/L carbenicillin and then cooled to 16 C prior to induction with 1.0 mM IPTG. Cells were harvested by centrifugation after 12 h of further growth and stored at −80 C. The cell pellet was resuspended in 50 mM Tris, pH 8.0, and 500 mM NaCl and sonicated on ice. After centrifugation, the supernatant was loaded onto a Ni-NTA agarose column (Qiagen). The column was washed with 50 mM Tris, pH 8.0, 500 mM NaCl, and 20 mM imidazole and then eluted with a step gradient in the same buffer with 250 mM imidazole. BAFF-containing fractions were pooled, thrombin was added, and the sample was dialyzed overnight against 20 mM Tris, pH 8.0, and 5 mM CaCl₂ at 4° C. The protein was further purified on a monoQ (Pharmacia) column and finally on an S-200 size exclusion column in 20 mM Tris, 150 mM NaCl, and 5 mM MgCl₂. The resulting BLyS protein was used as described below.

Peptide synthesis. Mini-BR3 (SEQ ID NO: 59) was synthesized as a C-terminal amide on a PERSEPTIVE BIOSYSTEMS® PIONEER™ Peptide Synthesizer (Applied Biosystems Inc.) using standard Fmoc chemistry. The side chain thiols of cysteines 19 and 32 were protected as trifluoroacetic acid (TFA)-stable acetamidomethyl (Acm) derivatives. Peptides were cleaved from the resin by treatment with 5% triisopropyl silane in TFA for 1.5-4 hr at room temperature. After removal of TFA by rotary evaporation, peptides were precipitated by addition of ethyl ether, then purified by reversed-phase HPLC (acetonitrile/H₂O/0.1% TFA). Peptide identity was confirmed by electrospray mass spectrometry. After lyophilization, the oxidized peptide was purified by HPLC. HPLC fractions containing reduced mini-BR3 were adjusted to a pH of 9 with NH₄OH; the disulfide between cysteines 24 and 35 was then formed by addition of a small excess of K₃Fe(CN)₆, and the oxidized peptide purified by HPLC. Acm groups were removed (with concomitant formation of the second disulfide) by treatment of the HPLC eluate with a small excess of I₂ over ˜4 h. The progress of the oxidation was monitored by analytical HPLC, and the final product was again purified by HPLC. Mini-BR3 (SEQ ID NO: 60) was amino-terminally biotinylated on the resin by reaction with a 10-fold molar excess of sulfo-NHS-biotin (Pierce Chemical, Co.). The biotinylated mini-BR3 was then cleaved from the resin and purified as described above for the unbiotinylated mini-BR3.

The following peptides, ECFDLLVRHWVPCGLLR (BLyS0048) (SEQ ID NO:14) and ECFDLLVRHWVPCGLLK (BLyS0095) (SEQ ID NO:62) were synthesized generally as follows. Peptides were synthesized on a RAININ® Symphony peptide synthesizer system using Rink amide resin and a threefold excess of 9-fluorenylmethoxycarbonyl (Fmoc) protected amino acid activated with 2-(1H-Benzotriazone-1-yl)-1,1,3,3-tetramethyluronium hexafluorophosphate (HBTU) in the presence of a fivefold excess of diisopropylethylamine (DIPEA). Amino acids were coupled twice at each position before deprotecting with a 20% solution of piperidine in dimethylformamide (DMF) and moving to the next residue. Washes between coupling steps were performed using dimethylacetamide (DMA). Following coupling of the final amino acid onto the peptide and its deprotection with 20% piperidine in DMF, the peptides were acylated at their amino terminus using 3 equivalents of acetic anhydride and 5 equivalents of DIPEA in DMA. Alternatively, the amino terminus was modified through acylation with 5-carboxyfluorescein, with (+)-biotin, or through reaction with another fluorophore or reporter molecule. The peptide was then cleaved from the resin through treatment with a solution of 95% trifluoroacetic acid (TFA) containing 2.5% water and 2.5% triisopropylsilane for 90 minutes. The volatiles were removed under reduced pressure, diethyl ether was added and the solids filtered off. The resulting precipitate was washed again with diethyl ether and the combined organics discarded. The washed solids were then washed successively with acetic acid, a 1:1 mixture of acetic acid and acetonitrile, a 1:1:1 mixture of acetic acid, acetonitrile and water, an 1:1:8 mixture of acetic acid, acetonitrile and water and finally with water. The combined washes were lyophilized and the resulting crude peptides purified using C18 reverse phase high performance liquid chromatography using a 30 minute 10% to 70% gradient of acetonitrile in water with 0.1% trifluoroacetic acid in each solvent at a flow rate of 15 milliliters per minute. Fractions containing the desired peptide were oxidized through addition of a saturated solution of iodine in acetic acid until the solution remained colored. This solution was then lyophilized. Finally, the lyophilized crude oxidized peptide was purified a second time under identical conditions and the fractions containing the desired peptide lyophilized. Some of the peptides were synthesized under identical conditions except that the synthesis was performed on a PERSEPTIVE BIOSYSTEMS® PIONEER™ Peptide Synthesizer (Applied Biosystems Inc.) using a fourfold excess of amino acid, coupling only once per residue.

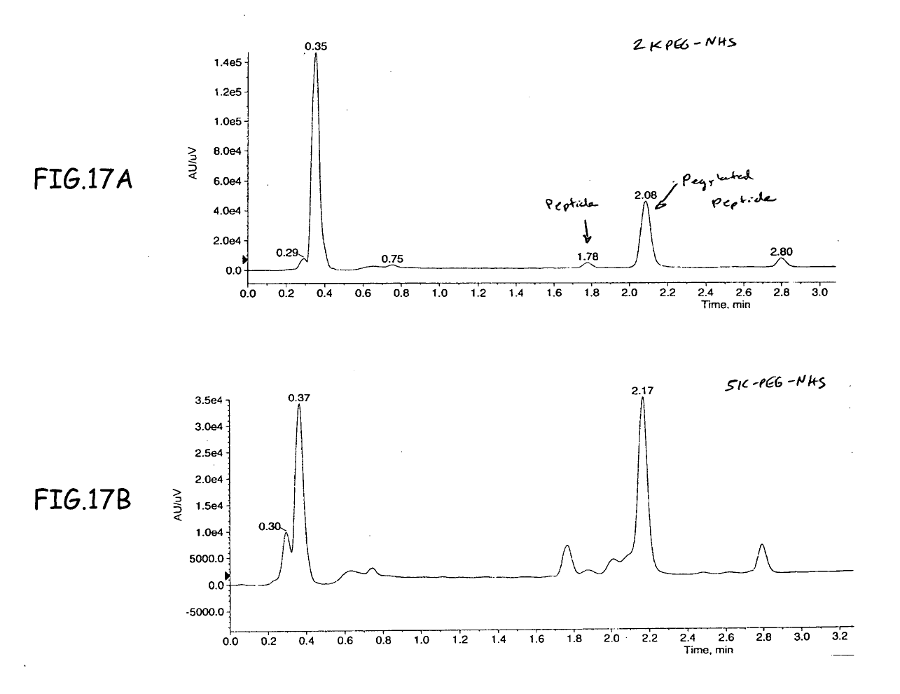

Conjugation of Polymers to Peptides. PEGylated 17-mer peptides were generated by using linear PEGs modified with N-hydroxysuccinimide chemistry (NHS) to react with primary amines (lysines and N-terminus). All PEG-NHS (PEG-SPA) reagents were purchased from Nektar Therapeutics, San Carlos, Calif. and stored under nitrogen at −70° C. The peptide was dissolved at 1 mg/mL in phosphate-buffer saline (PBS). To 0.4 mL aliquots of the peptide solution was added solid keg-SPA, keg-SPA, or 20 kPEG-SPA. Enough solid was added to obtain a 3:1 molar ratio of PEG-SPA to peptide. These solutions were incubated at room temperature for 1 hour and then the progress of the reaction was analyzed by reverse phase analytical HPLC on a 50 μL portion of the solution. The PEG addition and incubation was repeated 2 times until all of the peptide had been modified as shown by HPLC (FIGS. 17A and 17B). The PEGylated peptides were tested for BLyS binding without further purification.

HPLC chromatographs for the 2k PEG-conjugate and 5k PEG-conjugate are presented in FIG. 17A and FIG. 17B, respectively. In FIG. 17A, the unconjugated peptide corresponds to the peak at 1.78 min. and the 2K PEG-peptide conjugate corresponds to the peak at 2.08 minutes. In FIG. 17B, the unconjugated peptide corresponds to the peak at 1.78 min. and the 5K PEG-peptide conjugate corresponds to the peak at 2.17 minutes. The 20K PEG-conjugate is also purified by similar methods. The ratio of PEG:peptide in the purified conjugated product is approximately 1:1.

Competitive Displacement ELISA. A 17-mer, Ac-ECFDLLVRHWVPCGLLR-NH₂ (SEQ ID NO:14) (“BLyS0048”) was synthesized as described above. ECFDLLVRHWVPCGLLK (BLyS0095) (SEQ ID NO:62) was synthesized and coupled to each of 2K, 5K and 20K PEG-NHS as described above. Nunc® Maxisorp 96-well plates were coated overnight at 4° C. with 100 μl of a 2 μg/ml solution of BLyS in carbonate buffer, pH 9.6. The plate was washed with PBS and blocked with 1% skim milk in PBS. Serial dilutions of mini-BR3 (SEQ ID NO:59) and the above 17-mer peptide and PEG-peptide conjugate were prepared in PBS/0.05% TWEEN® 20 containing 3 ng/ml biotinylated mini-BR3. After washing with PBS/0.05% TWEEN®, 100 μl/well of each dilution was transferred and incubated for 1 hour at room temperature. The plate was washed with PBS/0.05% TWEEN® and incubated 15 min with 100 μl/well of 0.1 U/ml Streptavidin-POD (Boehringer Mannheim) in PBS/0.05% TWEEN®. After washing with PBS/0.05% TWEEN® followed by PBS, the plate was incubated 5 min with 100 μl PBS substrate solution containing 0.8 mg/ml OPD (Sigma) and 0.01% H₂O₂. The reaction was quenched with 100 μl/well 1M H₃PO₄ and the plate read at 490 nm. IC₅₀ values were determined by a four-parameter fit of the competitive displacement ELISA signal. The equation is: y=m1+(m2−m1)/(1+m0/m4)ˆm3, where m1 is the absorbance at infinite competitor concentration, m2 is the absorbance for no added competitor, m3 is the slope of the curve near the midpoint, m4 is the IC50 value and m0 is the concentration of competitor, peptide in this case. The concentration of biotinylated mini-BR3 was about 10 pM. The concentration of initial stock solution of mini-BR3 was determined by quantitative amino acid analysis.

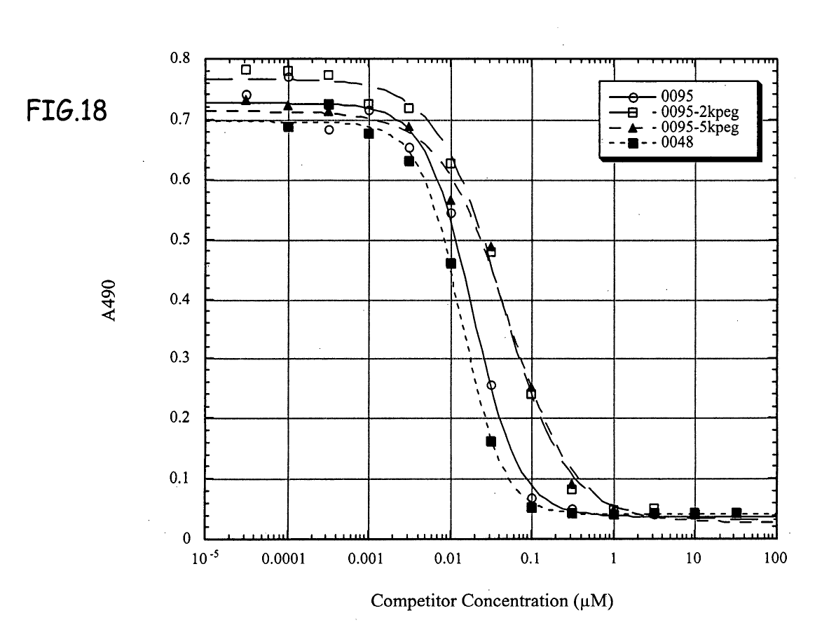

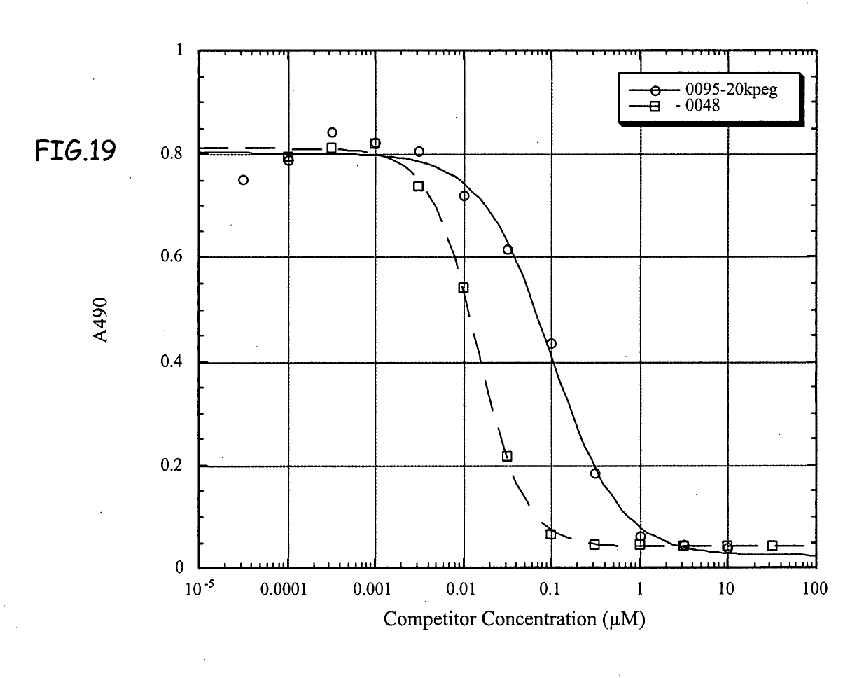

Results. The four-parameter fit of the competitive displacement ELISA signals of FIG. 18 provided IC50 values for: BLyS0095 (SEQ ID NO:62) of 19 nM, BLyS0048 (SEQ ID NO:14) of 14 nM and BLyS0095-2 kPEG conjugate of 43 nM, and BLyS0095-5 kPEG conjugate of 51 nM using this assay. Similarly, the fit of the competitive displacement ELISA signals of FIG. 19 provided IC50 values for BLyS0095-20 kPEG conjugate of 99 nM and BLyS0048 (SEQ ID NO:14) of 15 nM.

The 17-mer peptide-PEG conjugates (2k, 5k and 20k) demonstrated binding ability for BLyS. The conjugation of PEG to BLyS0095 (SEQ ID NO:62) did not significantly reduce its binding affinity as compared to similar unconjugated peptides.

1. A polypeptide comprising the sequence of Formula I:
X₁-C_{N}-X₃-D-X₅-L-X₇-X₈-X₉-X₁₀-X₁₁-X₁₂-C_{T}-X₁₄-X₁₅-X₁₆-X₁₇ (Formula I) (SEQ ID NO:1)
wherein X₁, X₃, X₅, X₇, X₈, X₉, X₁₀, X₁₁, X₁₂, X₁₄, X₁₅ and X₁₇ are any amino acid except cysteine; and
wherein X₁₆ is an amino acid selected from the group consisting of L, F, I and V;
wherein the polypeptide does not comprise a cysteine within seven amino acid residues N-terminal to C_{N} (cysteine N terminal) and C-terminal to C_{T} (cysteine C terminal) of Formula I;
wherein C_{N} and C_{T} are joined by disulfide bonding;
wherein the conformation of X₅LX₇X₈ forms a type I beta turn structure with the center of the turn between L and X₇; and
wherein X₈ has a positive value for the dihedral angle phi.

2. The polypeptide according to claim 1 wherein, X₁₀ is selected from the group consisting of W, F, V, L, I, Y, M and a non-polar amino acid. (SEQ ID NO:2).

3. The polypeptide according to claim 1, wherein X₁₀ is W. (SEQ ID NO:3).

4. The polypeptide according to claim 1, wherein the X₃ is an amino acid selected from the group consisting of M, V, L, 1, Y, F, W and a non-polar amino acid. (SEQ ID NO:4).

5. The polypeptide according to claim 1, wherein X₅ is selected from the group consisting of V, L, P, S, I, A and R. (SEQ ID NO:5).

6. The polypeptide according to claim 1, wherein the X₇ is selected from the group consisting of V, T, I and L. (SEQ ID NO:6).

7. The polypeptide according to claim 1, wherein the X₇ is not T or I (SEQ ID NO:7).

8. The polypeptide according to claim 1, wherein the X₈ is selected from the group consisting of any R, K, G, N, H and all D-amino acids. (SEQ ID NO:8).

9. The polypeptide according to claim 1, wherein X₉ is selected from the group consisting of H, K, A, R and Q. (SEQ ID NO:9).

10. The polypeptide according to claim 1, wherein the X₁₁ is selected from the group consisting of I and V. (SEQ ID NO:10).

11. The polypeptide according to claim 1, wherein the X₁₂ is selected from the group consisting of P, A, D, E and S. (SEQ ID NO:11).

12. The polypeptide according to claim 1, wherein the X₁₆ is L. (SEQ ID NO:12).

13. A polypeptide comprising the sequence of Formula II:
X₁-C_{N}-X₃-D-X₅-L-V-X₈-X₉-W-X₁₁-X₁₂-C_{T}-X₁₄-X₁₅-L-X₁₇ (Formula II) (SEQ ID NO:18)
wherein X₁, X₃, X₅, X₈, X₉, X₁₀, X₁₁, X₁₂, X₁₄, X₁₅ and X₁₇ are any amino acid except cysteine;
wherein the polypeptide does not comprise a cysteine within seven amino acid residues N-terminal to C_{N} (cysteine N terminal) and C-terminal to C_{T} (cysteine C terminal) of Formula II; and
wherein C_{N} and C_{T} are joined by disulfide bonding.

14. The polypeptide according to claim 13, wherein the conformation of X₅-L-V-X₈ forms a type I beta turn structure with the center of the turn between L and V; and
wherein X₈ has a positive value for the dihedral angle phi.

15. The polypeptide according to claim 13, wherein X₁, X₃, X₅, X₈, X₉, X₁₄, X₁₅ and X₁₇ are selected from a group of amino acids consisting of L, P, H, R, I, T, N, S, V, A, D, and G. (SEQ ID NO:19).

16. The polypeptide according to claim 13, wherein the X₃ is an amino acid selected from the group consisting of Norleucine, M, V, L, I, Y, F, W, and a non-polar amino acid. (SEQ ID NO:20).

17. The polypeptide according to claim 13, wherein X₅ is selected from the group consisting of V, L, P, S, I, A and R. (SEQ ID NO:21).

18. The polypeptide according to claim 13, wherein the X₈ is selected from the group consisting of R, K, G, N, H and all D-amino acids. (SEQ ID NO:22).

19. The polypeptide according to claim 13, wherein X₉ is selected from the group consisting of H, K, A, R and Q. (SEQ ID NO:23).

20. The polypeptide according to claim 13, wherein the X₁₁ is selected from the group consisting of I and V. (SEQ ID NO:24).

21. The polypeptide according to claim 13, wherein the X₁₂ is selected from the group consisting of P, A, D, E and S. (SEQ ID NO:25).

22. A polypeptide comprising an amino acid sequence of Formula III:
E-C_{N}-F-D-X₅-L-V-X₈-X₉-W-V-X₁₂-C_{T}-X₁₄-X₁₅-X₁₆-X₁₇ (Formula III) (SEQ ID NO:26)
wherein X₅, X₈, X₉, X₁₂, X₁₄, X₁₅ and X₁₇ are any amino acid except cysteine;
wherein X₁₆ is an amino acid selected from the group consisting of L, F, I and V;
wherein the polypeptide does not comprise a cysteine within seven amino acid residues N-terminal to C_{N} (cysteine N terminal) and C-terminal to C_{T} (cysteine C terminal) of Formula III; and
wherein C_{N} and C_{T} are joined by disulfide bonding.

23. The polypeptide according to claim 22, wherein the conformation forms a type I beta turn structure with the center of the turn between L and V; and
wherein V has a positive value for the dihedral angle phi.

24. The polypeptide according to claim 22, wherein X₅, X₈, X₉, X₁₂, X₁₄, X₁₅ and X₁₇ are selected from the group consisting of L, P, H, R, I, T, N, S, V, A, D, and G. (SEQ ID NO:27).

25. The polypeptide according to claim 22, wherein X₅ is L and X₈ is R. (SEQ ID NO:28).

26. The polypeptide according to claim 22, wherein X₉ is selected from the group consisting of H, K, A, S, R and Q. (SEQ ID NO:29).

27. The polypeptide according to claim 22, wherein X₁₂ is selected from the group consisting of P, A, D, E and S. (SEQ ID NO:30).

28. The polypeptide according to claim 22, wherein X₁₂ is P. (SEQ ID NO:31).

29. The polypeptide according to claim 22, wherein X₁₆ is L. (SEQ ID NO:32).

30. The polypeptide according to claim 22, wherein the sequence of Formula III is selected from the group consisting of ECFDLLVRAWVPCSVLK (SEQ ID NO:13), ECFDLLVRHWVPCGLLR (SEQ ID NO:14), ECFDLLVRRWVPCEMLG (SEQ ID NO:15), ECFDLLVRSWVPCHMLR (SEQ ID NO:16) and ECFDLLVRHWVACGLLR (SEQ ID NO:17).

31. A polypeptide comprising a polypeptide sequence selected from the group consisting of: SEQ ID NO:13 through SEQ ID NO:17 and SEQ ID NO: 62 through SEQ ID NO:137.

32. The polypeptides according to claim 1, wherein the polypeptide comprises additional sequences N-terminal, C-terminal or both N-terminal and C-terminal to a polypeptide sequence of Formula I wherein the additional sequences are heterologous to a BR3 polypeptide.

33. The polypeptide according to claim 1, wherein the sequence of Formula I is a sequence fused or conjugated to an immunoadhesion protein.

34. The polypeptide according to claim 1, wherein the sequence of Formula I is a sequence fused or conjugated to an antibody.

35. The polypeptide according to claim 34 wherein the antibody is selected from the group consisting of a F(ab) antibody, F(ab′)₂ antibody and a scFv antibody.

36. The polypeptide according to claim 34, wherein the antibody is selected from the group consisting of a humanized antibody and a multi-specific antibody.

37. The polypeptide according to claim 1, wherein the polypeptide is conjugated to an agent selected from the group consisting of a growth inhibitory agent, a cytotoxic agent, a detection agent, an agent that improves the bioavailability of the polypeptide and an agent that improves the half-life of the polypeptide.

38. The polypeptide according to claim 37, wherein said cytotoxic agent is selected from the group consisting of a toxin, an antibiotic and a radioactive isotope.

39. A nucleic acid molecule encoding the polypeptide according to claim 1.

40. A vector comprising the nucleic acid molecule according to claim 39.

41. A host cell comprising the nucleic acid molecule according to claim 39.

42. A method for producing a polypeptide comprising culturing a host cell comprising the vector according to claim 40 under conditions suitable for expressing the polypeptide from the vector.

43. The polypeptide according to claim 1, wherein said polypeptide is produced in bacteria.

44. The polypeptide according to claim 1, wherein said polypeptide is produced in CHO cells.

45. A composition comprising the polypeptide according to claim 1, optionally further comprising a physiologically acceptable carrier.

46. A method for detecting an inhibitor of BLyS binding to BR3 in vitro comprising detecting an inhibitor that prevents the polypeptide according to claim 1 from binding to BLyS.

47. A method for inhibiting BLyS binding to BR3 in a mammal comprising administering the polypeptide according to claim 1 in an amount effective to inhibit binding between BLyS and BR3 in the mammal.

48. (canceled)

49. A method for treating an immune-related condition in a mammal in need of treatment therefor comprising treating the mammal with a therapeutically effective amount of the polypeptide according to claim 1.

50. The method according to claim 48, wherein the immune related disease is selected from the group consisting of rheumatoid arthritis, multiple sclerosis and systemic lupus erythematosis.

51. A method for treating a cancer in a mammal in need of treatment therefor comprising treating the mammal with a therapeutically effective amount of the polypeptide according to claim 1.

52. The method according to claim 50, wherein said cancer is selected from the group consisting of leukemia, lymphoma and myeloma.

53. The method according to claim 50 wherein a therapeutically effective amount of an anti-CD20 antibody is also administered to the mammal.

54. The method according to claim 50, wherein the anti-CD20 antibody is a RITUXAN® antibody.

55. The polypeptide according to claim 1, wherein the polypeptide is conjugated to at least one non-proteinaceous polymer.

56. The polypeptide according to claim 55 wherein the non-proteinaceous polymer is a hydrophilic, synthetic polymer.

57. The polypeptide according to claim 55 wherein the non-proteinaceous polymer is polyethylene glycol.

58. The polypeptide according to claim 55 wherein the non-proteinaceous polymer is selected from the group consisting of: 2K-PEG, 5K-PEG, and 20K-PEG.

59. A polypeptide comprising at least two peptides selected from the group consisting of: SEQ ID NO:13 through SEQ ID NO:17 and SEQ ID NO: 62 through SEQ ID NO:137.

60. The polypeptide according to claim 59 wherein the peptides are the same sequence and the peptides are connected by a linker.

61. The polypeptide according to claim 59 wherein the peptides are different sequences and the peptides are connected by a linker.

62. The polypeptide according to claim 59, wherein the peptides linked together comprise a formula: PP1-L1-PP1-L2-PP1, wherein PP1 is a peptide selected from the group of claim 59 and L1 and L2 are linker sequences that are different in sequence.

63. The polypeptide according to claim 13, wherein the polypeptide comprises additional sequences N-terminal, C-terminal or both N-terminal and C-terminal to a polypeptide sequence of Formula II, wherein the additional sequences are heterologous to a BR3 polypeptide.

64. A nucleic acid molecule encoding the polypeptide according to claim 13.

65. A vector comprising the nucleic acid molecule according to claim 64.

66. A method for producing a polypeptide comprising culturing a host cell comprising the vector according to claim 65 under conditions suitable for expressing the polypeptide from the vector.

67. A composition comprising the polypeptide according to claim 13, optionally further comprising a physiologically acceptable carrier.

68. A method for inhibiting BLyS signaling in a mammal comprising administering the polypeptide according to claim 13 in an amount effective to inhibit binding between BLyS and BR3 in the mammal.

69. A method for treating an immune-related condition in a mammal in need of treatment therefor comprising treating the mammal with a therapeutically effective amount of the polypeptide according to claim 13.

70. The method according to claim 69, wherein the immune related disease is selected from the group consisting of rheumatoid arthritis, multiple sclerosis and systemic lupus erythematosis.

71. A method for treating a cancer in a mammal in need of treatment therefor comprising treating the mammal with a therapeutically effective amount of the polypeptide according to claim 13.

72. The method according to claim 71, wherein said cancer is selected from the group consisting of leukemia, lymphoma and myeloma.

73. The method according to claim 71 wherein a therapeutically effective amount of an anti-CD20 antibody is also administered to the mammal.

74. The polypeptide according to claim 13, wherein the polypeptide is conjugated to at least one non-proteinaceous polymer.

75. The polypeptide according to claim 74 wherein the non-proteinaceous polymer is polyethylene glycol.

76. The polypeptide according to claim 75 wherein the non-proteinaceous polymer is selected from the group consisting of: 2K-PEG, 5K-PEG, and 20K-PEG.

77. The polypeptide according to claim 74, wherein the peptides linked together comprise a formula: PP1-L1-PP1-L2-PP1, wherein PP1 is a peptide of claim 13 and L1 and L2 are linker sequences that are different in sequence.

78. The polypeptide according to claim 22, wherein the polypeptide comprises additional sequences N-terminal, C-terminal or both N-terminal and C-terminal to a polypeptide sequence of Formula III, wherein the additional sequences are heterologous to a BR3 polypeptide.

79. A nucleic acid molecule encoding the polypeptide according to claim 22.

80. A vector comprising the nucleic acid molecule according to claim 79.

81. A method for producing a polypeptide comprising culturing a host cell comprising the vector according to claim 80 under conditions suitable for expressing the polypeptide from the vector.

82. A composition comprising the polypeptide according to claim 22, optionally further comprising a physiologically acceptable carrier.

83. A method for inhibiting BLyS signaling in a mammal comprising administering the polypeptide according to claim 22 in an amount effective to inhibit binding between BLyS and BR3 in the mammal.

84. A method for treating an immune-related condition in a mammal in need of treatment therefor comprising treating the mammal with a therapeutically effective amount of the polypeptide according to claim 22.

85. The method according to claim 84, wherein the immune related disease is selected from the group consisting of rheumatoid arthritis, multiple sclerosis and systemic lupus erythematosis.

86. A method for treating a cancer in a mammal in need of treatment therefor comprising treating the mammal with a therapeutically effective amount of the polypeptide according to claim 22.

87. The method according to claim 86, wherein said cancer is selected from the group consisting of leukemia, lymphoma and myeloma.

88. The method according to claim 85, wherein a therapeutically effective amount of an anti-CD20 antibody is also administered to the mammal.

89. The polypeptide according to claim 22, wherein the polypeptide is conjugated to at least one non-proteinaceous polymer.

90. The polypeptide according to claim 89, wherein the non-proteinaceous polymer is polyethylene glycol.

91. The polypeptide according to claim 90, wherein the non-proteinaceous polymer is selected from the group consisting of: 2K-PEG, 5K-PEG, and 20K-PEG.


SEQUENCE LISTING (216 sequences)

SEQ ID NO: 1 - protein, 17 aa, Artificial Sequence
  Synthetic peptide sequences and Formula I
XCXDXLXXXXXXCXXXX

SEQ ID NO: 2 - protein, 17 aa, Artificial Sequence
  Synthetic peptide sequences and Formula I
XCXDXLXXXXXXCXXXX

SEQ ID NO: 3 - protein, 17 aa, Artificial Sequence
  Synthetic peptide sequences and Formula I
XCXDXLXXXXXXCXXXX

SEQ ID NO: 4 - protein, 17 aa, Artificial Sequence
  Synthetic peptide sequences and Formula I
XCXDXLXXXXXXCXXXX

SEQ ID NO: 5 - protein, 17 aa, Artificial Sequence
  Synthetic peptide sequences and Formula I
XCXDXLXXXXXXCXXXX

SEQ ID NO: 6 - protein, 17 aa, Artificial Sequence
  Synthetic peptide sequences and Formula I
XCXDXLXXXXXXCXXXX

SEQ ID NO: 7 - protein, 17 aa, Artificial Sequence
  Synthetic peptide sequences and Formula I
XCXDXLXXXXXXCXXXX

SEQ ID NO: 8 - protein, 17 aa, Artificial Sequence
  Synthetic peptide sequences and Formula I
XCXDXLXXXXXXCXXXX

SEQ ID NO: 9 - protein, 17 aa, Artificial Sequence
  Synthetic peptide sequences and Formula I
XCXDXLXXXXXXCXXXX

SEQ ID NO: 10 - protein, 17 aa, Artificial Sequence
  Synthetic peptide sequences and Formula I
XCXDXLXXXXXXCXXXX

SEQ ID NO: 11 - protein, 17 aa, Artificial Sequence
  Synthetic peptide sequences and Formula I
XCXDXLXXXXXXCXXXX

SEQ ID NO: 12 - protein, 17 aa, Artificial Sequence
  Synthetic peptide sequences and Formula I
XCXDXLXXXXXXCXXXX

SEQ ID NO: 13 - protein, 17 aa, Artificial Sequence
  Clone 44; BLyS0051
ECFDLLVRAWVPCSVLK

SEQ ID NO: 14 - protein, 17 aa, Artificial Sequence
  BLyS0048
ECFDLLVRHWVPCGLLR

SEQ ID NO: 15 - protein, 17 aa, Artificial Sequence
  Clone 2, 6,11, 14, 24, 28, 34, 36, 40, 42, 46, 47
ECFDLLVRRWVPCEMLG

SEQ ID NO: 16 - protein, 17 aa, Artificial Sequence
  Clone 7, 9, 10, 20, 25, 29
ECFDLLVRSWVPCHMLR

SEQ ID NO: 17 - protein, 17 aa, Artificial Sequence [sequence omitted]

SEQ ID NO: 18 - protein, 17 aa, Artificial Sequence
  Synthetic peptide sequences and Formula II
XCXDXLVXXWXXCXXLX

SEQ ID NO: 19 - protein, 17 aa, Artificial Sequence
  Synthetic peptide sequences and Formula II
XCXDXLVXXWXXCXXLX

SEQ ID NO: 20 - protein, 17 aa, Artificial Sequence
  Synthetic peptide sequences and Formula II
XCXDXLVXXWXXCXXLX

SEQ ID NO: 21 - protein, 17 aa, Artificial Sequence
  Synthetic peptide sequences and Formula II
XCXDXLVXXWXXCXXLX

SEQ ID NO: 22 - protein, 17 aa, Artificial Sequence
  Synthetic peptide sequences and Formula II
XCXDXLVXXWXXCXXLX

SEQ ID NO: 23 - protein, 17 aa, Artificial Sequence
  Synthetic peptide sequences and Formula II
XCXDXLVXXWXXCXXLX

SEQ ID NO: 24 - protein, 17 aa, Artificial Sequence
  Synthetic peptide sequences and Formula II
XCXDXLVXXWXXCXXLX

SEQ ID NO: 25 - protein, 17 aa, Artificial Sequence
  Synthetic peptide sequences and Formula II
XCXDXLVXXWXXCXXLX

SEQ ID NO: 26 - protein, 17 aa, Artificial Sequence
  Synthetic peptide sequences and Formula III
ECFDXLVXXWVXCXXXX

SEQ ID NO: 27 - protein, 17 aa, Artificial Sequence
  Synthetic peptide sequences and Formula III
ECFDXLVXXWVXCXXXX

SEQ ID NO: 28 - protein, 17 aa, Artificial Sequence
  Synthetic peptide sequences and Formula III
ECFDXLVXXWVXCXXXX

SEQ ID NO: 29 - protein, 17 aa, Artificial Sequence
  Synthetic peptide sequences and Formula III
ECFDXLVXXWVXCXXXX

SEQ ID NO: 30 - protein, 17 aa, Artificial Sequence
  Synthetic peptide sequences and Formula III
ECFDXLVXXWVXCXXXX

SEQ ID NO: 31 - protein, 17 aa, Artificial Sequence
  Synthetic peptide sequences and Formula III
ECFDXLVXXWVXCXXXX

SEQ ID NO: 32 - protein, 17 aa, Artificial Sequence
  Synthetic peptide sequences and Formula III
ECFDXLVXXWVXCXXXX

SEQ ID NO: 33 - DNA, 858 bp, Homo sapiens
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtga

SEQ ID NO: 34 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 35 - DNA, 595 bp, Homo sapiens
cgtcggcaccatgaggcgagggccccggagcctgcggggcagggacgcgccagcccccacgccctgcgtcccggccgagtgcttcgacctgctggtccgccactgcgtggcctgcgggctcctgcgcacgccgcggccgaaaccggccggggccagcagccctgcgcccaggacggcgctgcagccgcaggagtcggtgggcgcgggggccggcgaggcggcgctgcccctgcccgggctgctctttggcgcccccgcgctgctgggcctggcactggtcctggcgctggtcctggtgggtctggtgagctggaggcggcgacagcggcggcttcgcggcgcgtcctccgcagaggcccccgacggagacaaggacgccccagagcccctggacaaggtcatcattctgtctccgggaatctctgatgccacagctcctgcctggcctcctcctggggaagacccaggaaccaccccacctggccacagtgtccctgtgccagccacagagctgggctccactgaactggtgaccaccaagacggccggccctgagcaacaatagcagggagccggcaggaggtggcccctgccc

SEQ ID NO: 36 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 37 - DNA, 1881 bp, Mus musculus
atgggcgccaggagactccggttccgaagccagaggagccgggacagctcggtgcccacccagtgcaatcagaccgagtgcttcgaccctctggtgagaaactgcgtgtcctgtgagctcttccacacgccggacactggacatacaagcagcctggagcctgggacagctctgcagcctcaggagggctccgcgctgagacccgacgtggcgctgctcgtcggtgcccccgcactcctgggactgatactggcgctgaccctggtgggtctagtgagtctggtgagctggaggtggcgtcaacagctcaggacggcctccccagacacttcagaaggagtccagcaagagtccctggaaaatgtctttgtaccctcctcagaaacccctcatgcctcagctcctacctggcctccgctcaaagaagatgcagacagcgccctgccacgccacagcgtcccggtgcccgccacagaactgggctccaccgagctggtgaccaccaagacagctggcccagagcaatagcagcagtggaggctggaacccagggatctctactgggcttgtggacttcacccaacagcttgggaaagaacttggcccttcagtgacggagtcctttgcctggggggcgaacccggcagaaccagacactacaggccacatgagattgcttttgtgttagctcttgacttgagaacgttccatttctgagatggtttttaagcctgtgtgccttcagatggttggatagacttgagggttgcatatttaatctctgtagtgagtcggagactggaaacttaatctcgttctaaaaattttggattactgggctggaggtatggctcagcagttcggtttgtgtgctgttctagccgaggactccagttgttcagcttcccggaactcagatctggcagcttaagaccacctgtcactccagcccctggaacatccttgcctccaaaggcaccagcactcatttgctctagagcacacacacacacacacacacacacacacacacacacacacatatgcatgcatgcacacttaaaaatgtcaaaattagcggctggagaaattcatggtcaacagcgcttactgtgattccagaggatgagagtttgattcccagaatgcactgcgggtggctcattactgagcataacttttgcttcaggggacctgatgcctctggacttcatgggcatctgtattcacgtgcacatcctacacacacacacacacacacacacagacatacacacacacacactcttttacaaatgataaaatataagataggcatggtggtacacacctttaatcccaacattggggaagcaaaggcaggcaggtaactgagttggaggccatcctggtctacatagcaagttccaggctaaccagagctaaatggtgagaccaagtctcaaaataatactccccccccaaaaaaaaaaaacttttaaattttgatttttttcttttattattattttttatattaatttcatggtgtttagaagtggtatacttagatggtgactaagaggaggtaaagccatcaggactgagcccctaacatacaaggagaaagcagagacaatgaacacgcccctctcctgctgtgtgccagctctggaccaccagccagagggcaatcatcagatgtgggccctagaaccttcagagccgaaagctaaatcaatctcatttctttgtaaagctatttagccttaggtgttttgttacggtgatataaaatggactaacacaggcactatgagtaagaagcttttctttgagctgggaaaggtactgttaaaccaaaattaatctgaataaaaaaaggctaaggggaagacactt

SEQ ID NO: 38 - protein, 175 aa, Mus musculus
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

SEQ ID NO: 39 - DNA, 891 bp, Homo sapiens
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctcct

SEQ ID NO: 40 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 41 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 42 - protein, 107 aa, Artificial Sequence
  Humanized 2H7.v16 VL
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 43 - protein, 107 aa, Artificial Sequence
  VLk SGI
DIQMTQSPSSLSASVGDRVTITCRASQSISNYLAWYQQKPKAPKLLIYAASSLESGVPSRFSGSGSGTDFTLTISSLQPEDFATYYCQQYNSLPWTFGQGTKVEIKR

SEQ ID NO: 44 - protein, 10 aa, Mus musculus
RASSSVSYMH

SEQ ID NO: 45 - protein, 7 aa, Mus musculus
APSNLAS

SEQ ID NO: 46 - protein, 9 aa, Mus musculus
QQWSFNPPT

SEQ ID NO: 47 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 48 - protein, 122 aa, Artificial Sequence [sequence omitted]

SEQ ID NO: 49 - protein, 119 aa, Artificial Sequence
  VH SGIII
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 50 - protein, 10 aa, Mus musculus
GYTFTSYNMH

SEQ ID NO: 51 - protein, 17 aa, Mus musculus
AIYPGNGDTSYNQKFKG

SEQ ID NO: 52 - protein, 13 aa, Mus musculus
VVYYSNSYWYFDV

SEQ ID NO: 53 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 54 - protein, 185 aa, Homo sapiens
MRRGPRSLRGRDAPAPTPCVPAECFDLLVRHCVACGLLRTPRPKPAGAASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 55 - protein, 175 aa, Rat
MGVRRLRVRSRRSRDSPVSTQCNQTECFDPLVRNCVSCELFYTPETRHASSLEPGTALQPQEGSGLRPDVALLFGAPALLGLVLALTLVGLVSLVGWRWRQQRRTASLDTSEGVQQESLENVFVPPSETLHASAPNWPPFKEDADNILSCHSIPVPATELGSTELVTTKTAGPEQ

SEQ ID NO: 56 - protein, 232 aa, Artificial Sequence
  humanized 2H7.v16 light chain
MGWSCIILFLVATATGVHSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 471 aa, Artificial Sequence
  Humanized 2H7.v16 Heavy chain
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 471 aa, Artificial Sequence
  Humanized 2H7.v31 Heavy chain
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 26 aa, Artificial Sequence
  mini-BR3
TPCVPAECFDLLVRHCVACGLLRTPR

SEQ ID NO: 60 - protein, 61 aa, Artificial Sequence
  human BR3-ECD
MRRGPRSLRGRDAPAPTPCVPAECFDLLVRHCVACGLLRTPRPKPAGASSPAPRTALQPQE

SEQ ID NO: 61 - protein, 7 aa, Artificial Sequence
  Linker
GGGSGGG

SEQ ID NO: 62 - protein, 17 aa, Artificial Sequence
  BLyS0095
ECFDLLVRHWVPCGLLK

SEQ ID NO: 63 - protein, 17 aa, Artificial Sequence
  Clone 1
ECFDLLVRQWVPCERIR

SEQ ID NO: 64 - protein, 17 aa, Artificial Sequence
  Clone 3
ECFDLLVRKWVPCQVLG

SEQ ID NO: 65 - protein, 17 aa, Artificial Sequence
  Clone 4
ECFDLLVRTWVECSLLN

SEQ ID NO: 66 - protein, 17 aa, Artificial Sequence
  Clone 5
ECFDLLVRSWVPCGTLM

SEQ ID NO: 67 - protein, 17 aa, Artificial Sequence
  Clone 8
ECFDLLVRTWVPCQAIL

SEQ ID NO: 68 - protein, 17 aa, Artificial Sequence
  Clone 12
ECFDLLVRAWVRCDMLL

SEQ ID NO: 69 - protein, 17 aa, Artificial Sequence
  Clone 13
ECFDLLVRGWVPCEKLM

SEQ ID NO: 70 - protein, 17 aa, Artificial Sequence
  Clone 15
ECFDLLVRAWVPCWLRL

SEQ ID NO: 71 - protein, 17 aa, Artificial Sequence
  Clone 16
ECFDLLVRRWVPCGLLR

SEQ ID NO: 72 - protein, 17 aa, Artificial Sequence
  Clone 17
ECFDLLVRRWVDCAFLH

SEQ ID NO: 73 - protein, 17 aa, Artificial Sequence
  Clone 18
ECFDLLVRSWVPCSSLG

SEQ ID NO: 74 - protein, 17 aa, Artificial Sequence
  Clone 19
ECFDLLVRTWVPCNVLX

SEQ ID NO: 75 - protein, 17 aa, Artificial Sequence
  Clone 21
ECFDLLVRRWVPCELLV

SEQ ID NO: 76 - protein, 17 aa, Artificial Sequence
  Clone 22
ECFDLLVRSWVPCYSLK

SEQ ID NO: 77 - protein, 17 aa, Artificial Sequence
  Clone 23
ECFDLLVRQWVSCQVFA

SEQ ID NO: 78 - protein, 17 aa, Artificial Sequence
  Clone 26
ECFDLLVRVWVPCSRLY

SEQ ID NO: 79 - protein, 17 aa, Artificial Sequence
  Clone 27
ECFDLLVRQWVPCGALG

SEQ ID NO: 80 - protein, 17 aa, Artificial Sequence
  Clone 30
ECFDLLVRAWVPCNELR

SEQ ID NO: 81 - protein, 17 aa, Artificial Sequence
  Clone 31
ECFDLLVREWVPCRILQ

SEQ ID NO: 82 - protein, 17 aa, Artificial Sequence
  Clone 32
ECFDLLVRRWVPCSWLL

SEQ ID NO: 83 - protein, 17 aa, Artificial Sequence
  Clone 33
ECFDLLVRRWVPCSLVK

SEQ ID NO: 84 - protein, 17 aa, Artificial Sequence
  Clone 35
ECFDLLVRQWVPCRALM

SEQ ID NO: 85 - protein, 17 aa, Artificial Sequence
  Clone 37
ECFDLLVRAWVPCSYLS

SEQ ID NO: 86 - protein, 17 aa, Artificial Sequence
  Clone 38
ECFDLLVRDWVPCSLLF

SEQ ID NO: 87 - protein, 17 aa, Artificial Sequence
  Clone 39
ECFDLLVRSWVPCTLLS

SEQ ID NO: 88 - protein, 17 aa, Artificial Sequence
  Clone 41
ECFDLLVRKWVPCSTFH

SEQ ID NO: 89 - protein, 17 aa, Artificial Sequence
  Clone 43
ECFDLLVRGWVPCSVLQ

SEQ ID NO: 90 - protein, 17 aa, Artificial Sequence
  Clone 45
ECFDLLVRQWVSCELLS

SEQ ID NO: 91 - protein, 17 aa, Artificial Sequence
  Clone 48
ECFDLLVRGWVDCSLLL

SEQ ID NO: 92 - protein, 17 aa, Artificial Sequence
  Clone 49
ECFDILVDRWVPCAILH

SEQ ID NO: 93 - protein, 17 aa, Artificial Sequence
  Clone 50
ECFDRLVGHWVPCAALI

SEQ ID NO: 94 - protein, 17 aa, Artificial Sequence
  Clone 52, 71
ECFDPLVARWVPCHLIN

SEQ ID NO: 95 - protein, 17 aa, Artificial Sequence
  Clone 53
ECFDPLVRVWVDCSILD

SEQ ID NO: 96 - protein, 17 aa, Artificial Sequence
  Clone 54
ECFDSLVNAWVPCSAIR

SEQ ID NO: 97 - protein, 17 aa, Artificial Sequence
  Clone 55
ECFDLLVNRWVDCRLLI

SEQ ID NO: 98 - protein, 17 aa, Artificial Sequence
  Clone 56
ECFDPLVRIWVACDRLA

SEQ ID NO: 99 - protein, 17 aa, Artificial Sequence
  Clone 57
ECFDPLVGRWVPCTLLH

SEQ ID NO: 100 - protein, 17 aa, Artificial Sequence
  Clone 58
ECFDLLVRAWVPCHLID

SEQ ID NO: 101 - protein, 17 aa, Artificial Sequence
  Clone 59
ECFDPLVGHWVPCSVLT

SEQ ID NO: 102 - protein, 17 aa, Artificial Sequence
  Clone 60
ECFDPLVNRWVDCVALH

SEQ ID NO: 103 - protein, 17 aa, Artificial Sequence
  Clone 61
ECFDRLVNLWVDCALLN

SEQ ID NO: 104 - protein, 17 aa, Artificial Sequence
  Clone 62
ECFDVLVSAWVDCARLN

SEQ ID NO: 105 - protein, 17 aa, Artificial Sequence
  Clone 63
ECFDSLVRLWVPCNLLR

SEQ ID NO: 106 - protein, 22 aa, Artificial Sequence
  Clone 64
ECFDPLVRHWVPCNLLRGAGSP

SEQ ID NO: 107 - protein, 17 aa, Artificial Sequence
  Clone 65
ECFDILVNAWVPCRVIG

SEQ ID NO: 108 - protein, 17 aa, Artificial Sequence
  Clone 66
ECFDRLVNRWVPCNLIV

SEQ ID NO: 109 - protein, 17 aa, Artificial Sequence
  Clone 67
ECFDRLVRAWVPCTALT

SEQ ID NO: 110 - protein, 17 aa, Artificial Sequence
  Clone 68
ECFDLLVRRWVPCHLIT

SEQ ID NO: 111 - protein, 17 aa, Artificial Sequence
  Clone 69
ECFDILVGRWVPCGLIH

SEQ ID NO: 112 - protein, 17 aa, Artificial Sequence
  Clone 70
ECFDPLVRDWVRCDILT

SEQ ID NO: 113 - protein, 17 aa, Artificial Sequence
  Clone 72
ECFDPLVRVWVPCTVLR

SEQ ID NO: 114 - protein, 17 aa, Artificial Sequence
  Clone 73
ECFDSLVRAWVPCGVLS

SEQ ID NO: 115 - protein, 17 aa, Artificial Sequence
  Clone 74
ECFDVLVHRWVPCGLIR

SEQ ID NO: 116 - protein, 17 aa, Artificial Sequence
  Clone 75
ECFDHLVRIWVPCTALA

SEQ ID NO: 117 - protein, 17 aa, Artificial Sequence
  Clone 76
ECFDTLVNAWVPCNLLD

SEQ ID NO: 118 - protein, 17 aa, Artificial Sequence
  Clone 77
ECFDRLVNGWVPCAVLH

SEQ ID NO: 119 - protein, 17 aa, Artificial Sequence
  Clone 78
ECFDRLVNAWVDCRLLA

SEQ ID NO: 120 - protein, 17 aa, Artificial Sequence
  Clone 79
ECFDLLVNDWVPCGAIT

SEQ ID NO: 121 - protein, 17 aa, Artificial Sequence
  Clone 80
ECFDALVRRWVDCSLLR

SEQ ID NO: 122 - protein, 17 aa, Artificial Sequence
  Clone 81
ECFDALVHRWVDCAVLG

SEQ ID NO: 123 - protein, 17 aa, Artificial Sequence
  Clone 82
ECFDVLVNAWVDCAVLR

SEQ ID NO: 124 - protein, 17 aa, Artificial Sequence
  Clone 83
ECFDGLVNAWVDCGLLR

SEQ ID NO: 125 - protein, 17 aa, Artificial Sequence
  Clone 84
ECFDPLVRHWVPCRALD

SEQ ID NO: 126 - protein, 17 aa, Artificial Sequence
  Clone 85
ECFDDLVRHWVPCDLLT

SEQ ID NO: 127 - protein, 17 aa, Artificial Sequence
  Clone 86
ECFDVLVRAWVPCRALT

SEQ ID NO: 128 - protein, 17 aa, Artificial Sequence
  Clone 87
ECFDILVNRWVPCGALT

SEQ ID NO: 129 - protein, 17 aa, Artificial Sequence
  Clone 88
ECFDDLVRNWVPCALLN

SEQ ID NO: 130 - protein, 17 aa, Artificial Sequence
  Clone 89
ECFDPLVNAWVPCAVLH

SEQ ID NO: 131 - protein, 17 aa, Artificial Sequence
  Clone 90
ECFDPLVLRWVPCSALH

SEQ ID NO: 132 - protein, 17 aa, Artificial Sequence
  Clone 91
ECFDALVHRWVPCDLLR

SEQ ID NO: 133 - protein, 17 aa, Artificial Sequence
  Clone 92
ECFDPLVRDWVPCDLIH

SEQ ID NO: 134 - protein, 17 aa, Artificial Sequence
  Clone 93
ECFDLLVNSWVPCSVIA

SEQ ID NO: 135 - protein, 17 aa, Artificial Sequence
  Clone 94
ECFDTLVRAWVPCSHLT

SEQ ID NO: 136 - protein, 17 aa, Artificial Sequence
  Clone 95
ECFDSLVRIWVPCGLID

SEQ ID NO: 137 - protein, 17 aa, Artificial Sequence
  Clone 96
ECFDSLVNAWVPCHVLT

SEQ ID NO: 138 - DNA, 75 bp, Artificial Sequence
  Clone 1
aatgcctatgcagaatgcttcgatctgctggttcgtcagtgggtgccgtgtgagcggatcaggggtggaggatcc

SEQ ID NO: 139 - DNA, 75 bp, Artificial Sequence
  Clone 2,16,11,14,24,28, 34,36,40,42,46,47
aatgcctatgcagaatgcttcgatctgctggttcgtcgctgggtgccgtgtgagatgttggggggtggaggatcc

SEQ ID NO: 140 - DNA, 75 bp, Artificial Sequence
  Clone 3
aatgcctatgcagaatgcttcgatctgctggttcgtaagtgggtgccctgtcaggtgttgggcggtggaggatcc

SEQ ID NO: 141 - DNA, 75 bp, Artificial Sequence
  Clone 4
aatgcctatgcagaatgcttcgatctgctggttcgtacctgggtggagtgttccttgttgaacggtggaggatcc

SEQ ID NO: 142 - DNA, 75 bp, Artificial Sequence
  Clone 5
aatgcctatgcagaatgcttcgatctgctggttcgttcgtgggtgccctgtggcaccttgatgggtggaggatcc

SEQ ID NO: 143 - DNA, 75 bp, Artificial Sequence
  Clone 7,9,10,20,25,29
aatgcctatgcagaatgcttcgatctgctggttcgttcctgggtgccgtgtcacatgctccggggtggaggatcc

SEQ ID NO: 144 - DNA, 75 bp, Artificial Sequence
  Clone 8
aatgcctatgcagaatgcttcgatctgctggttcgtacgtgggtgccctgtcaggcgatcttgggtggaggatcc

SEQ ID NO: 145 - DNA, 75 bp, Artificial Sequence
  Clone 12
aatgcctatgcagaatgcttcgatctgctggttcgtgcgtgggtgaggtgtgacatgttgctgggtggaggatcc

SEQ ID NO: 146 - DNA, 75 bp, Artificial Sequence
  Clone 13
aatgcctatgcagaatgcttcgatctgctggttcgtggctgggtgccgtgtgaaaagctcatgggtggaggatcc

SEQ ID NO: 147 - DNA, 75 bp, Artificial Sequence
  Clone 15
aatgcctatgcagaatgcttcgatctgctggttcgtgcgtgggtgccctgttggttgagactgggtggaggatcc

SEQ ID NO: 148 - DNA, 75 bp, Artificial Sequence
  Clone 16
aatgcctatgcagaatgcttcgatctgctggttcgtcgctgggtgccctgtgggctgctgaggggtggaggatcc

SEQ ID NO: 149 - DNA, 75 bp, Artificial Sequence
  Clone 17
aatgcctatgcagaatgcttcgatctgctggttcgtcggtgggtggactgtgcgttcttgcacggtggaggatcc

SEQ ID NO: 150 - DNA, 75 bp, Artificial Sequence
  Clone 18
aatgcctatgcagaatgcttcgatctgctggttcgttcctgggtgccgtgttccagcctgggcggtggaggatcc

SEQ ID NO: 151 - DNA, 75 bp, Artificial Sequence
  Clone 19
aatgcctatgcagaatgcttcgatctgctggttcgtacgtgggtgccgtgtaacgtgttgtagggtggaggatcc

SEQ ID NO: 152 - DNA, 75 bp, Artificial Sequence
  Clone 21
aatgcctatgcagaatgcttcgatctgctggttcgtaggtgggtgccgtgtgagttgctcgtgggtggaggatcc

SEQ ID NO: 153 - DNA, 75 bp, Artificial Sequence
  Clone 22
aatgcctatgcagaatgcttcgatctgctggttcgtagctgggtgccctgttacagcctgaagggtggaggatcc

SEQ ID NO: 154 - DNA, 75 bp, Artificial Sequence
  Clone 23
aatgcctatgcagaatgcttcgatctgctggttcgtcagtgggtgtcgtgtcaggtgttcgcgggtggaggatcc

SEQ ID NO: 155 - DNA, 75 bp, Artificial Sequence
  Clone 26
aatgcctatgcagaatgcttcgatctgctggttcgtgtctgggtgccgtgttccaggctctacggtggaggatcc

SEQ ID NO: 156 - DNA, 75 bp, Artificial Sequence
  Clone 27
aatgcctatgcagaatgcttcgatctgctggttcgtcagtgggtgccctgtggggcgctcgggggtggaggatcc

SEQ ID NO: 157 - DNA, 75 bp, Artificial Sequence
  Clone 30
aatgcctatgcagaatgcttcgatctgctggttcgtgcctgggtgccctgtaacgagctgcgcggtggaggatcc

SEQ ID NO: 158 - DNA, 75 bp, Artificial Sequence
  Clone 31
aatgcctatgcagaatgcttcgatctgctggttcgtgagtgggtgccgtgtcggatcttgcagggtggaggatcc

SEQ ID NO: 159 - DNA, 75 bp, Artificial Sequence
  Clone 32
aatgcctatgcagaatgcttcgatctgctggttcgtcggtgggtgccgtgttcctggctgctgggtggaggatcc

SEQ ID NO: 160 - DNA, 75 bp, Artificial Sequence
  Clone 33
aatgcctatgcagaatgcttcgatctgctggttcgtaggtgggtgccgtgtagcctggtcaagggtggaggatcc

SEQ ID NO: 161 - DNA, 75 bp, Artificial Sequence
  Clone 35
aatgcctatgcagaatgcttcgatctgctggttcgtcagtgggtgccgtgtagggcgctgatgggtggaggatcc

SEQ ID NO: 162 - DNA, 75 bp, Artificial Sequence
  Clone 37
aatgcctatgcagaatgcttcgatctgctggttcgtgcgtgggtgccctgttcgtacctgtcgggtggaggatcc

SEQ ID NO: 163 - DNA, 75 bp, Artificial Sequence
  Clone 38
aatgcctatgcagaatgcttcgatctgctggttcgtgactgggtgccgtgttcgctgctcttcggtggaggatcc

SEQ ID NO: 164 - DNA, 75 bp, Artificial Sequence
  Clone 39
aatgcctatgcagaatgcttcgatctgctggttcgttcctgggtgccctgtacgttgctctcgggtggaggatcc

SEQ ID NO: 165 - DNA, 75 bp, Artificial Sequence
  Clone 41
aatgcctatgcagaatgcttcgatctgctggttcgtaagtgggtgccctgttcgacgttccacggtggaggatcc

SEQ ID NO: 166 - DNA, 75 bp, Artificial Sequence
  Clone 43
aatgcctatgcagaatgcttcgatctgctggttcgtgggtgggtgccctgttcggtcttgcagggtggaggatcc

SEQ ID NO: 167 - DNA, 75 bp, Artificial Sequence
  Clone 44
aatgcctatgcagaatgcttcgatctgctggttcgtgcgtgggtgccgtgttcggtcttgaagggtggaggatcc

SEQ ID NO: 168 - DNA, 75 bp, Artificial Sequence
  Clone 45
aatgcctatgcagaatgcttcgatctgctggttcgtcagtgggtgtcgtgtgagctgctctccggtggaggatcc

SEQ ID NO: 169 - DNA, 75 bp, Artificial Sequence
  Clone 48
aatgcctatgcagaatgcttcgatctgctggttcgtgggtgggtggactgtagcctgttgttgggtggaggatcc

SEQ ID NO: 170 - DNA, 75 bp, Artificial Sequence
  Clone 49
aatgcctatgcagaatgcttcgatatcctggttgaccgctgggtgccctgtgccatcctccacggtggaggatcc

SEQ ID NO: 171 - DNA, 75 bp, Artificial Sequence
  Clone 50
aatgcctatgcagaatgcttcgatcgcctggttggccactgggtgccctgtgccgccctcatcggtggaggatcc

SEQ ID NO: 172 - DNA, 75 bp, Artificial Sequence
  Clone 52,71
aatgcctatgcagaatgcttcgatcccctggttgcccgctgggtgccctgtcacctcatcaacggtggaggatcc

SEQ ID NO: 173 - DNA, 75 bp, Artificial Sequence
  Clone 53
aatgcctatgcagaatgcttcgatcccctggttcgcgtctgggtggactgtagcatcctcgacggtggaggatcc

SEQ ID NO: 174 - DNA, 75 bp, Artificial Sequence
  Clone 54
aatgcctatgcagaatgcttcgatagcctggttaacgcctgggtgccctgtagcgccatccgcggtggaggatcc

SEQ ID NO: 175 - DNA, 75 bp, Artificial Sequence
  Clone 55
aatgcctatgcagaatgcttcgatctcctggttaaccgctgggtggactgtcgcctcctcatcggtggaggatcc

SEQ ID NO: 176 - DNA, 75 bp, Artificial Sequence
  Clone 56
aatgcctatgcagaatgcttcgatcccctggttcgcatctgggtggcctgtgaccgcctcgccggtggaggatcc

SEQ ID NO: 177 - DNA, 75 bp, Artificial Sequence
  Clone 57
aatgcctatgcagaatgcttcgatcccctggttggccgctgggtgccctgtaccctcctccacggtggaggatcc

SEQ ID NO: 178 - DNA, 75 bp, Artificial Sequence
  Clone 58
aatgcctatgcagaatgcttcgatctcctggttcgcgcctgggtgccctgtcacctcatcgacggtggaggatcc

SEQ ID NO: 179 - DNA, 75 bp, Artificial Sequence
  Clone 59
aatgcctatgcagaatgcttcgatcccctggttggccactgggtgccctgtagcgtcctcaccggtggaggatcc

SEQ ID NO: 180 - DNA, 75 bp, Artificial Sequence
  Clone 60
aatgcctatgcagaatgcttcgatcccctggttaaccgctgggtggactgtgtcgccctccacggtggaggatcc

SEQ ID NO: 181 - DNA, 75 bp, Artificial Sequence
  Clone 61
aatgcctatgcagaatgcttcgatcgcctggttaacctctgggtggactgtgccctcctcaacggtggaggatcc

SEQ ID NO: 182 - DNA, 75 bp, Artificial Sequence
  Clone 62
aatgcctatgcagaatgcttcgatgtcctggttagcgcctgggtggactgtgcccgcctcaacggtggaggatcc

SEQ ID NO: 183 - DNA, 75 bp, Artificial Sequence
  Clone 63
aatgcctatgcagaatgcttcgatagcctggttcgcctctgggtgccctgtaacctcctccgcggtggaggatcc

SEQ ID NO: 184 - DNA, 99 bp, Artificial Sequence
  Clone 64
aatgcctatgcagaatgcttcgatcccctggttcgccactgggtgccctgtaacctcctccgcggtaaagatctggcaggttcaccaggtggaggatcc

SEQ ID NO: 185 - DNA, 75 bp, Artificial Sequence
  Clone 65
aatgcctatgcagaatgcttcgatatcctggttaacgcctgggtgccctgtcgcgtcatcggcggtggaggatcc

SEQ ID NO: 186 - DNA, 75 bp, Artificial Sequence
  Clone 66
aatgcctatgcagaatgcttcgatcgcctggttaaccgctgggtgccctgtaacctcatcgtcggtggaggatcc

SEQ ID NO: 187 - DNA, 75 bp, Artificial Sequence
  Clone 67
aatgcctatgcagaatgcttcgatcgcctggttcgcgcctgggtgccctgtaccgccctcaccggtggaggatcc

SEQ ID NO: 188 - DNA, 75 bp, Artificial Sequence
  Clone 68
aatgcctatgcagaatgcttcgatctcctggttcgccgctgggtgccctgtcacctcatcaccggtggaggatcc

SEQ ID NO: 189 - DNA, 75 bp, Artificial Sequence
  Clone 69
aatgcctatgcagaatgcttcgatatcctggttggccgctgggtgccctgtggcctcatccacggtggaggatcc

SEQ ID NO: 190 - DNA, 75 bp, Artificial Sequence
  Clone 70
aatgcctatgcagaatgcttcgatcccctggttcgcgattgggtgcgctgtgacatcctcaccggtggaggatcc

SEQ ID NO: 191 - DNA, 75 bp, Artificial Sequence
  Clone 72
aatgcctatgcagaatgcttcgatcccctggttcgcgtctgggtgccctgtaccgtcctccgcggtggaggatcc

SEQ ID NO: 192 - DNA, 75 bp, Artificial Sequence
  Clone 73
aatgcctatgcagaatgcttcgatagcctggttcgcgcctgggtgccctgtggcgtcctcagcggtggaggatcc

SEQ ID NO: 193 - DNA, 75 bp, Artificial Sequence
  Clone 74
aatgcctatgcagaatgcttcgatgtcctggttcaccgctgggtgccctgtggcctcatccgcggtggaggatcc

SEQ ID NO: 194 - DNA, 75 bp, Artificial Sequence
  Clone 75
aatgcctatgcagaatgcttcgatcacctggttcgcatctgggtgccctgtaccgccctcgccggtggaggatcc

SEQ ID NO: 195 - DNA, 75 bp, Artificial Sequence
  Clone 76
aatgcctatgcagaatgcttcgataccctggttaacgcctgggtgccctgtaacctcctcgacggtggaggatcc

SEQ ID NO: 196 - DNA, 75 bp, Artificial Sequence
  Clone 77
aatgcctatgcagaatgcttcgatcgcctggttaacggctgggtgccctgtgccgtcctccacggtggaggatcc

SEQ ID NO: 197 - DNA, 75 bp, Artificial Sequence
  Clone 78
aatgcctatgcagaatgcttcgatcgcctggttaacgcctgggtggactgtcgcctcctcgccggtggaggatcc

SEQ ID NO: 198 - DNA, 75 bp, Artificial Sequence
  Clone 79
aatgcctatgcagaatgcttcgatctcctggttaacgactgggtgccctgtggcgccatcaccggtggaggatcc

SEQ ID NO: 199 - DNA, 75 bp, Artificial Sequence
  Clone 80
aatgcctatgcagaatgcttcgatgccctggttcgccgctgggtggactgtagcctcctccgcggtggaggatcc

SEQ ID NO: 200 - DNA, 75 bp, Artificial Sequence
  Clone 81
aatgcctatgcagaatgcttcgatgccctggttcaccgctgggtggactgtgccgtcctcggcggtggaggatcc

SEQ ID NO: 201 - DNA, 75 bp, Artificial Sequence
  Clone 82
aatgcctatgcagaatgcttcgatgtcctggttaacgcctgggtggactgtgccgtcctccgcggtggaggatcc

SEQ ID NO: 202 - DNA, 75 bp, Artificial Sequence
  Clone 83
aatgcctatgcagaatgcttcgatggcctggttaacgcctgggtggactgtggcctcctccgcggtggaggatcc

SEQ ID NO: 203 - DNA, 75 bp, Artificial Sequence
  Clone 84
aatgcctatgcagaatgcttcgatcccctggttcgccactgggtgccctgtcgcgccctcgacggtggaggatcc

SEQ ID NO: 204 - DNA, 75 bp, Artificial Sequence
  Clone 85
aatgcctatgcagaatgcttcgatgacctggttcgccactgggtgccctgtgacctcctcaccggtggaggatcc

SEQ ID NO: 205 - DNA, 75 bp, Artificial Sequence
  Clone 86
aatgcctatgcagaatgcttcgatgtcctggttcgtgcctgggtgccctgtcgcgccctcaccggtggaggatcc

SEQ ID NO: 206 - DNA, 75 bp, Artificial Sequence
  Clone 87
aatgcctatgcagaatgcttcgatatcctggttaaccgctgggtgccctgtggcgccctcaccggtggaggatcc

SEQ ID NO: 207 - DNA, 75 bp, Artificial Sequence
  Clone 88
aatgcctatgcagaatgcttcgatgacctggttcgcaactgggtgccctgtgccctcctcaacggtggaggatcc

SEQ ID NO: 208 - DNA, 75 bp, Artificial Sequence
  Clone 89
aatgcctatgcagaatgcttcgatcccctggttaacgcctgggtgccctgtgccgtcctccacggtggaggatcc

SEQ ID NO: 209 - DNA, 75 bp, Artificial Sequence
  Clone 90
aatgcctatgcagaatgcttcgatcccctggttctccgctgggtgccctgtagcgccctccacggtggaggatcc

SEQ ID NO: 210 - DNA, 75 bp, Artificial Sequence
  Clone 91
aatgcctatgcagaatgcttcgatgccctggttcaccgctgggtgccctgtgacctcctccgcggtggaggatcc

SEQ ID NO: 211 - DNA, 75 bp, Artificial Sequence
  Clone 92
aatgcctatgcagaatgcttcgatcccctggttcgcgactgggtgccctgtgacctcatccacggtggaggatcc

SEQ ID NO: 212 - DNA, 75 bp, Artificial Sequence
  Clone 93
aatgcctatgcagaatgcttcgatctcctggttaacagctgggtgccctgtagcgtcatcgccggtggaggatcc

SEQ ID NO: 213 - DNA, 75 bp, Artificial Sequence
  Clone 94
aatgcctatgcagaatgcttcgataccctggttcgcgcctgggtgccctgtagccacctcaccggtggaggatcc

SEQ ID NO: 214 - DNA, 75 bp, Artificial Sequence
  Clone 95
aatgcctatgcagaatgcttcgatagcctggttcgcatctgggtgccctgtggcctcatcgacggtggaggatcc

SEQ ID NO: 215 - DNA, 75 bp, Artificial Sequence
  Clone 96
aatgcctatgcagaatgcttcgatagcctggttaacgcctgggtgccctgtcacgtcctcaccggtggaggatcc

SEQ ID NO: 216 - protein, 17 aa, Artificial Sequence
  17mer region of native human BR3
ECFDLLVRHCVACGLLR